(19) United States
(12) Patent Application Publication
(10) Pub. No.: US 2022/0367016 A1
(43) Pub. Date: Nov. 17, 2022

(54) DYNAMIC HEALTH RECORDS

(71) Applicant: Leonard H. Ginsburg, Merion, PA (US)

(72) Inventor: Leonard H. Ginsburg, Merion, PA (US)

(21) Appl. No.: 17/686,194

(22) Filed: Mar. 3, 2022

Related U.S. Application Data
(63) Continuation-in-part of application No. 16/802,547, filed on Feb. 26, 2020.
Application No. 16/802,547 is a continuation-in-part of application No. 17/187,843, filed on Feb. 28, 2021.
Application No. 17/187,843 is a continuation-in-part of application No. 17/008,586, filed on Aug. 31, 2020.
Application No. 17/008,586 is a continuation-in-part of application No. 17/035,648, filed on Sep. 28, 2020.
(60) Provisional application No. 62/810,868, filed on Feb. 26, 2019.
(60) Provisional application No. 63/127,840, filed on Dec. 18, 2020.
(60) Provisional application No. 63/116,684, filed on Nov. 20, 2020.
(60) Provisional application No. 62/893,688, filed on Aug. 29, 2019.
(60) Provisional application No. 62/907,410, filed on Sep. 27, 2019.
(60) Provisional application No. 62/983,350, filed on Feb. 28, 2020.
(60) Provisional application No. 62/987,165, filed on Mar. 9, 2020.
(60) Provisional application No. 63/026,547, filed on May 18, 2020.
(60) Provisional application No. 62/907,410, filed on Sep. 27, 2019.

(51) Int. Cl. G16H 10/60

(52) U.S. Cl. CPC G16H 10/60

(57) ABSTRACT

A computer implemented method of creating display configurations includes generating, via a medical record dashboard system, a dashboard display comprising at least a first visible panel of one or more visible panels having selected information corresponding to information stored in one or more medical record databases related to different respective medical services. A configuration menu associated with the first panel is displayed and display configuration parameters selected by a first user for a first display configuration are received in response to user interaction with the display configuration menu. The first panel is configured based on the first configuration and the display configuration parameters of the first configuration are stored. A selection of the first display configuration by a second user may be received via a configuration sharing panel. The first display configuration is retrieved as is data defined by the first display configuration. The first display configuration is then applied to a second panel selected by the second user.

### CROSS REFERENCE TO RELATED APPLICATIONS

This application is a continuation-in-part of U.S. patent application Ser. No. 16/802,547, filed Feb. 26, 2020, which application claims priority to 62/810,868, filed Feb. 26, 2019; this application is also a continuation-in-part of U.S. patent application Ser. No. 17/187,843, filed Feb. 28, 2021, which application claims the benefit of priority to U.S. Provisional Application Ser. No. 63/127,840, filed Dec. 18, 2020 and U.S. Provisional Application Ser. No. 63/116,684, filed Nov. 20, 2020, and is a continuation-in-part of U.S. patent application Ser. No. 17/008,586, filed Aug. 31, 2020, which application claims the benefit of priority to U.S. Provisional Application Ser. No. 62/893,688, filed Aug. 29, 2019; U.S. Provisional Application Ser. No. 62/907,410, filed Sep. 27, 2019; U.S. Provisional Application Ser. No. 62/983,350, filed Feb. 28, 2020. U.S. Provisional Application Ser. No. 62/987,165, filed Mar. 9, 2020; and U.S. Provisional Application Ser. No. 63/026,547 filed May 18, 2020; this application is also a continuation-in-part of U.S. patent application Ser. No. 17/035,648, filed Sep. 28, 2020, which claims the benefit of priority to U.S. Provisional Application Ser. No. 62/907,410, filed Sep. 27, 2019, which applications and publications are incorporated herein by reference in their entirety.

### BACKGROUND

The third most common cause of death in the United States is medical error. Needed is a mechanism to help assist doctors when placing orders to allow them to see relevant data, medical services, guidelines, and what is most important to consider emphasized and displayed. Important data relevant to placing an order should be visible at the time of creation of the order. When Doctors prescribe or create orders, they do so in a vacuum, not certain what the order they placed actually looks like against other relevant data, and cannot identify an order placed immediately or in the future is in fact for example is the correct body part in relation to other relevant information that may impact decision making. If the order could be seen in context, medical errors could be reduced. The importance of also displaying relevant data when a medical professional is viewing and interpreting diagnostic tests as well documenting and creating assessment and plans for care of the patient, is also vital in the delivery of efficient, accurate care and preventing errors. Among the most common reasons for malpractice claims are patients missing appointments or scheduled medical services. Medical professionals have no efficient way of knowing or prioritizing those patients most at risk or knowing when future appointments are scheduled and with which provider or medical service. What is needed is an intuitive display system that helps the doctor identify patients at risk with missed medical services and a system that automatically notifies the correct user and even the patient, so important medical services are not delayed and performed.

Caregivers are often called upon to make rapid life and death decisions based on a patient's condition in the context of a medical history as presented, for example, in an Electronic Health Record's (“EHR”). However, the visual display systems for EHR's are difficult to understand and require the user to move through multiple screens, interfaces, and menus to obtain the disparate information needed to make a medical decision. This creates great difficulties when caring for multiple patients in a busy practice and is compounded when different doctors provide care for the same patient. Moreover, the complex interfaces associated with EMRs are particularly problematic at the point of care as they slow caregivers down and distract them from meaningful face-time, caring for patients. Communication and sharing care for a particular patient between multiple health care providers has become more difficult. Now, rather than a fax or short dictated medical summary, caregivers are sending voluminous amounts of information often filled with error from click mistakes, right, left confusion and cut and paste functionality. EHR also has no organized way to correlate associated data over time nor share information across different EHR's, and non-integrated systems.

Furthermore, no system displays clinical and examination findings, medications actually taken by the patient, procedures, and diagnostic tests in a way that a user can discover at a glance if treatment is effective. No system, provides the ability for the user to visualize in context to allow them to double check that the orders and medications they are placing, are in fact the exact correct ones they intend to order. There is no system that displays, correlates, and highlights interrelated data points that can make a difference in the life of each patient. When information is displayed in a flow chart in an EHR, the presentations are not able to be adjusted manually and dynamically for an individual patient. Now that former paper medical records have been digitalized, data is very difficult to find. Medical care and the associated data dispersed in the computer has become so complex that what is needed is the type of intelligent, actionable visual display system that in context automatically adjusts the presentation, sorts, compresses and highlights the data the user needs to be able to make a medical decision and visualize the cause-and-effect of treatment.

In health care, EHR systems, and practice and image management systems borrow dashboards and displays from other fields. These displays often have time or date in one direction, with width or height consistent and variable factors that are being shown or measured in the other direction. The importance of certain occurrences in time may deserve more or less emphasis. Time spent does not always equate with work effort, impact of findings, or results.

While these traditional methods of evaluating data may work for some fields, these displays are sorely lacking in the medical field, which demands an entire new approach to all existing displays, flow charts, spreadsheets, and dashboards. In medicine a particular date of service could have an occurrence with much greater significance than another date. One date might be a routine office examination and another cancer discovered. An encounter with one provider, can be much more impactful than another. Both dates of service may have common features such as a particular clinical measurement. However, what is needed is a way to express the intensely different occurrences, so that at a glance with limited space and time, the critical information for a particular patient is conveyed. Unfortunately, EMR's, if they have a flow chart, it Is borrowed from displays in other fields, and simply displays data in similar ways used outside of medicine. Existing spreadsheets such as excel, may work for assisting accountants or even building an airplane. Once one plane is built, the next can be built the exact same way. Replicating even one human being has never been accomplished and no two people are the same nor react to disease and medical care in the exact same way.

What is needed is an entirely new display approach to presenting, organizing, and measuring data tailored for the human being. A simple, elegant solution that enables caregivers to synthesize information and populate and document a chart and even display orders as created when seeing a patient. A single presentation that enables a caregiver to identify medical problems and errors through data visualization, where data is presented and displayed in an intuitive, easy to view manner.

### SUMMARY

A computer implemented method of creating display configurations includes generating, via a medical record dashboard system, a dashboard display comprising at least a first visible panel of one or more visible panels having selected information corresponding to information stored in one or more medical record databases related to different respective medical services. A configuration menu associated with the first panel is displayed and display configuration parameters selected by a first user for a first display configuration are received in response to user interaction with the display configuration menu. The first panel is configured based on the first configuration and the display configuration parameters of the first configuration are stored. A selection of the first display configuration by a second user may be received via a configuration sharing panel. The first display configuration is retrieved as is data defined by the first display configuration. The first display configuration is then applied to a second panel selected by the second user.

In various embodiments, a data command center visual display system and associated methods operate to display data on a display from multiple data sources and allowing navigation amongst the data without leaving the display of the visual display system. Numerous technical issues rooted in computer technology must be solved for the data to be presented to the visual display system so that the data may be displayed in the command center using a single display interface. For example, the visual display system must provide access to the requisite health information systems and third-party support services whereby the data may be accessed, processed, and presented without unacceptable delay. Also, the display data must be collected and ordered to facilitate the various combinations of the data into respective display panels that may be navigated on the interface. For example, it is desirable for the data to be configured in a task-based or specialty-specific display configuration for use by physicians, for example. To do this, various features in prior art systems needed to be acquired and combined in a new way to facilitate access to the features without having to navigate away from the display. For example, conventional EMR systems provide interfaces to third party prescription ordering systems but require the user the navigate to another system and away from the EMR interface. Accessing ordering panels without leaving the display becomes particularly difficult where the display space is limited as is the case for many physicians who use portable display devices and mobile computers. The structural embodiments described herein address these technical issues to generate the Dynamic health Records actionable display system embodiments described herein.

In exemplary embodiments, such a data command center visual display system in accordance with the present principles includes a patient database that stores patient identification information, patient insurance information, patient medical history information, a computer readable storage medium having stored thereon instructions thereon, and a processor that executes the instructions to perform operations including creating a plurality of adjustable display panels configured to display predetermined combinations of the patient identification information, patient insurance information, patient medical history information, and creating a patient flowsheet that integrates the patient medical history information into a table that presents the patient's medical history by visit to at least one physician with respective procedures or actions performed during each visit represented as first icons identifying the procedure or action performed and second icons enabling selection of a new procedure or action, where the first and second icons provide links to associated patient medical information and ordering display panels that may be accessed without leaving the display. In response to selection of the second icon by a user of the visual display system, an ordering display panel is presented to the display in addition to the adjustable display panels and patient flowsheet. The desired procedures or actions may be ordered from the ordering display panels while relevant portions of the patient's medical history are still visible on the display screen. The scope of the claims also contemplates corresponding methods performed by the visual display system and users thereof.

In exemplary embodiments, the ordering display panel comprises an ePrescribing panel for ordering medication or a medical procedure ordering panel for ordering a medical procedure. By way of example, the medical procedure ordering panel for ordering a medical procedure may further provide a link to the quality reporting panel that displays quality reporting metrics and/or peer data related to the procedure that is being ordered. All of such ordering display panels are configured in the context of the display to conserve display space so that the ordering display panel may be displayed while still being able to view the medical history data, for example.

In other exemplary embodiments, the ordering display panel comprises an imaging order panel for ordering a medical image of the patient or a lab order panel for ordering a lab test of the patient. In still other embodiments, instructions are provided that when executed create an image icon in an adjustable display panel and/or the patient flowsheet that, when selected by the user of the visual data system, opens a display window for viewing of one or more images without leaving the display.

In other exemplary embodiments, the visual display system incorporates financial data with the patient medical history data into the display panels. Such a visual display system includes a patient database that stores patient identification information, patient insurance information, patient medical history information, and patient payment information, a computer readable storage medium having stores thereon instructions thereon, and a processor that executes the instructions to perform operations including creating a plurality of adjustable display panels configured to display predetermined combinations of the patient identification information, patient insurance information, patient medical history information, and patient payment information, and creating a patient flowsheet that integrates the patient medical history information and patient payment information into a table that presents the patient's medical history by visit to at least one physician with respective procedures or actions performed during each visit represented as first icons identifying the procedure or action performed and second icons indicating whether the procedure or action has been paid for in part or in full, the first and second icons providing links to associated patient medical history information and/or patient payment information. In response to selection by a user of the visual display system, the adjustable display panels and patient flowsheet are moved into a task-based or specialty-specific display configuration such that the patient identification information, patient insurance information, patient medical history information, and patient payment information may be accessed without leaving the display. The task-based or specialty-specific display configuration is then presented to the display. In exemplary embodiments, selection of the first icons or second icons open display windows to associated medical history data and/or financial data and overlay a portion of the display with the display windows whereby the associated medical history data and/or financial data may be viewed by the user of the visual display system while the adjustable display panels and the patient flowsheet are displayed in a background on the display. Throughout this description, it will be appreciated that all financial data in the system, including costs to patient, is compartmentalized such that no user may see financial details for users or organizations not authorized in accordance with applicable policies and law. Also, the scope of the claims also contemplates corresponding methods performed by the visual display system and users thereof.

The visual display system includes a number of features that enable accessing information on the display. For example, third icons are provided in the patient flowsheet or display panels that include links to compliance information about compliance with insurance guidelines and/or good clinical practice guidelines for a procedure or action associated with each third icon. In exemplary embodiments, the compliance information includes aggregated medical treatment guidelines and an overview outlining similarities and differences amongst different medical treatment guidelines making up the aggregated medical treatment guidelines. The aggregated medical treatment guidelines may include information related to recommended follow-up with the patient, information related to procedures permitted or prevented by the patient's insurance or contra-indications, and information relating to proper billing for the procedure or action associated with a third icon selected from the patient flowsheet or display panels. In exemplary embodiments, the visual display system provides access to a clinical decision support system that uses a rules engine and/or natural language processing to aggregate the medical treatment guidelines and to generate the overview outlining similarities and differences amongst different medical treatment guidelines making up the aggregated medical treatment guidelines. The clinical decision support system and/or natural language processing system may further compare medical data to notice patterns, errors and anomalies in different entries or notes, find discrepancies in payments, alert the user of the visual display system about inconsistent medical documentation or improper orders, speed up the process of complying with regulations, alert the user of the visual display system that a plan or order is inconsistent with a preferred practice plan for a patient, or warn the user of the visual display system that billing certain procedures might not be covered. The natural language processing system may also be accessed parse notes in the patient flowsheet or display panels for potential ICD10 codes or alternative diagnosis.

The visual display system also includes a display configuration that enables users of the visual display system to order medications, diagnostic tests, images, procedures, and the like directly from the patient flowsheet or display panel. For example, an icon or link in the patient flowsheet or display panel may include an ePrescribing panel for ordering medication or a medical procedure ordering panel for ordering a medical procedure. The medical procedure ordering panel may further include a link to a quality reporting panel that displays quality reporting metrics and/or peer data related to the procedure that is being ordered. In other embodiments, an icon or link in the patient flowsheet or display panel may include an imaging order panel for ordering a medical image of the patient or a lab order panel for ordering a lab test of the patient. In still other embodiments, an image icon is provided in an adjustable display panel and/or the patient flowsheet that, when selected by the user of the visual data system, opens a display window for viewing of one or more images without leaving the display screen. In other embodiments, an alert icon is provided in an adjustable display panel and/or the patient flowsheet that, when selected by the user of the visual data system, opens an alert message without leaving the display. In still other embodiments, one of the display panels may be configured to accept today's visit notes from the user of the visual display system in connection with a patient visit for storage for access with other data of the one display panel.

In still other embodiments, data input by the user of the visual display system may trigger auto-population of information in the adjustable display panels and patient flowsheet and auto-population of the patient's medical record in an electronic medical record system. In the exemplary embodiments, the auto-population occurs without the user of the video display system leaving the display.

In other embodiments, new clinical information for the patient is provided to a diagnosis evaluation algorithm for comparison of the new clinical information with previous corresponding clinical information for the patient to determine whether the new clinical information is indicative of an improvement or worsening of the patient's medical condition. The visual display system further generates diagnosis indicators providing a visual representation of an improvement of a medical problem, disease, or symptom, or a worsening of a medical problem, disease, or symptom as a result of taking a particular medication or undergoing a particular medical procedure and displays the diagnosis indicators in the adjustable display panels and/or the patient flowsheet.

Other embodiments of the visual display system allows for increased speed of data presentation by a local database that stores a subset of patient identification information, patient insurance information, patient medical history information, and patient payment information, where the subset includes the patient identification information, patient insurance information, patient medical history information, and patient payment information for patients having an appointment within a predetermined time window.

The visual display system in exemplary embodiments includes interfaces to an external health information system and third-party service systems. In exemplary embodiments, the external health information system includes at least one of an electronic health records system, Electronic Medical records, a practice management system, a health information exchange, a picture archive and communications system, a clearing house/billing system, Image management systems, diagnostic equipment, and a laboratory system. On the other hand, the third party service systems may include one or more of an ePrescribing system, an insurance verification/referral/pre-authorization system, a system for establishing medical necessity by verifying that a procedure or medication is associated with a correct diagnostic code such as an ICD10 code or other current code supporting its use, a clinical services pricing and location system, a claim status checking system, services in support of the National Correct Coding Initiative, services to proactively ensure claims are coded correctly to prevent issues in billing, claims compliance services that evaluate claims against National Coverage Determination (NCD) and Local Coverage Determination (LCD) guidelines as well as local insurance regulations to establish and document medical necessity, a natural language processing system, and artificial intelligence/cognitive systems that provide clinical decision support.

In exemplary embodiments, the patient identification information, patient insurance information, patient medical history information, and patient payment information is stored in the patient database in transactional tables that capture clinical and billing data and reporting tables where data is aggregated for a particular physician, practice, health system or other entity. Each table uses a surrogate primary key that is a unique value within the table used to identify a row that is not directly tied to data in that row. In the exemplary embodiments, XML code moves and stores different display panel and flowsheet views. The XML code further identifies a collection of panels and tabs, wherein within each panel is a panel ID that links the panel to a tab, the panel's position, and whether or not the panel is stacked with another panel. The XML code may also set up the display panels and patient flowsheet on the display by, for example, identifying a collection of columns and, for each column, a name of the column along with a data source. The display panels so configured are presented to the display for selection and display panel frames on the display screen are manipulated for receiving selected display panels.

In other exemplary embodiments, the patient flowsheet is organized around patient medical information corresponding to a particular disease state and/or procedures and/or insurance coverage and/or actions for treating the particular disease state.

The patient database may also be adapted to include patient medical history information from a plurality of medical care providers whereby the patient flowsheet may be adapted to include medical history information from more than one medical care provider in order to provide shared treatment of the patient in the patient flowsheet. In other embodiments, a summary table may be provided that illustrates everything the user of the visual display system has done for each patient in a particular time frame or for each patient having a particular disease state in a particular time frame. The summary table may also include information from other medical care providers who are providing shared treatment of the patient. If financial data, cost, charge, payment is on the summary table with the medical data, this data is compartmentalized such that no user may see financial details for users or organizations not authorized in accordance with applicable policies and law.

In yet other embodiments, a data command center visual display system is provided that presents dynamic data to a display. The command center visual display system includes a plurality of adjustable display panels configured to display predetermined combinations of patient identification information and patient medical information. A patient flowsheet is created that includes a table that presents the patient's medical information by medical service, medical procedure, diagnostic test, medication, and diagnosis that is prescribed, ordered, performed, or selected during respective encounters with at least one medical care provider. In response to selection by a user, at least two adjustable display panels containing medical information relating to one or more patients in the patient flowsheet are presented to the display in a single view. The user may edit or move the medical information or the patient identification information within the display panels while the display panels are simultaneously open.

In some embodiments, a method for rules-based data display in a data command center including a medical records dashboard including one or more windows including information received or derived from at least one patient database, the medical records dashboard comprising a display, using the one or more windows, of at least one of medical services, clinical data, examination findings, diagnostic tests, and the procedures performed on one or more patients, the one or more windows comprising a plurality of data fields, including at least one dynamic data field, for displaying the information received or derived from the at least one patient database, wherein the at least one of the medical services, the clinical data, the examination findings, the diagnostic tests, and the procedures are arranged in rows or columns on the display according to at least one of a time and a date that the medical services, the clinical data, the examination findings, the diagnostic tests and the procedures were performed on the one or more patients, the method includes receiving patient-related data from the at least one patient database, comparing the received patient-related data with configuration rules to determine which portions of the received patient-related data are to be displayed in data fields of the medical records dashboard, identifying dynamic data fields of the at least one dynamic data field of the medical records dashboard that are determined to not have any patient-related data to display as collapsed data fields, displaying patient-related data in the data fields of the medical records dashboard in accordance with the configuration rules and dynamic data fields of the medical records dashboard identified as collapsed data fields.

In some embodiments, a data command center visual display system that displays data on a display includes a computing device comprising at least one processor, a non-transitory computer-readable medium, having stored thereon, software instructions that when executed by the at least one processor of the computing device, cause the computing device to perform operations comprising at least, linking to and receiving patient related medical records including patient data from at least one patient data source, and displaying a medical records dashboard including one or more windows, the medical record dashboard capable of displaying, using the one or more windows, patient data from at least one patient data source including at least one of medical services, clinical data, examination findings, diagnostic tests, and the procedures performed on one or more patients, the one or more windows comprising a plurality of data fields, including at least one dynamic data field, for displaying the information received or derived from the at least one patient database, wherein the at least one of the medical services, the clinical data, the examination findings, the diagnostic tests, and the procedures are arranged in rows or columns on the one or more windows according to at least one of a time and a date that the medical services, the clinical data, the examination findings, the diagnostic tests and the procedures were performed on the one or more patients, wherein a display of patient data in the medical records dashboard is determined by: comparing the patient data with configuration rules to determine which portions of the patient data are to be displayed in the data fields of the medical records dashboard, identifying dynamic data fields of the at least one dynamic data field of the medical records dashboard that are determined to not have patient data to display as collapsed data fields, and displaying patient data in the data fields of the medical records dashboard in accordance with the configuration rules and dynamic data fields of the medical records dashboard identified as collapsed data fields. The terms computer-readable medium, machine readable medium, and storage device do not include carrier waves to the extent carrier waves are deemed too transitory. Storage can also include networked storage, such as a storage area network (SAN).

In some embodiments, a method for unique patient identification of a subject patient in a data command center including patient-related data received or derived from at least one patient database includes collecting patient-related data having different data classifications from the at least one patient database, assigning a level of accuracy score for each of the patient-related data of the different classifications, adding, the level of accuracy scores for each of the patient-related data of the different classifications, comparing a total of the added level of accuracy scores to a previously determined matching threshold, if the total of the added level of accuracy scores exceeds the matching threshold, establishing an identification of the subject patient, and if the total of the added level of accuracy scores does not exceed the matching threshold, collecting additional patient-related data and returning to the assigning phase.

In some embodiments, a data command center visual display system for determining a unique patient identification includes a computing device comprising at least one processor, a non-transitory computer-readable medium, having stored thereon, software instructions that when executed by the at least one processor of the computing device, cause the computing device to perform operations comprising at least: linking to and receiving patient related medical records including patient data from at least one patient data source, collecting patient-related data having different data classifications from the at least one patient database, assigning a level of accuracy score for each of the patient-related data of the different classifications, adding, the level of accuracy scores for each of the patient-related data of the different classifications, comparing a total of the added level of accuracy scores to a previously determined matching threshold, if the total of the added level of accuracy scores exceeds the matching threshold, establishing an identification of the subject patient, and if the total of the added level of accuracy scores does not exceed the matching threshold, collecting additional patient-related data and returning to the assigning.

In some embodiments, a method for medication management and display in a data command center comprising one or more windows for display and including information received or derived from at least one patient database, the data command center displaying, using the one or more windows, at least one of medical services, clinical data, examination findings, diagnostic tests, and procedures performed on one or more patients, the one or more windows comprising a plurality of data fields for displaying the information received or derived from the at least one patient database, wherein the at least one of the medical services, the clinical data, the examination findings, the diagnostic tests, and the procedures are arranged in on the one or more windows according to at least one of a time and a date that the medical services, the clinical data, the examination findings, the diagnostic tests and the procedures were performed on the one or more patients, includes determining, from at least one of the information received or derived from the at least one patient database and the at least one of the medical services, the clinical data, the examination findings, the diagnostic tests, and the procedures, medications prescribed or administered to the one or more patients, generating a respective graphical representation for each of the determined prescribed or medications prescribed or administered to the one or more patients, and displaying at least one generated, respective graphical representation of at least one medication administered to a patient in the at least one or more windows in context with at least one of the information received or derived from the at least one patient database and the at least one of the medical services, the clinical data, the examination findings, the diagnostic tests, and the procedures, wherein the at least one generated, respective graphical representation of the at least one medication administered to the patient is arranged in on the one or more windows according to at least one of the times and the dates that the at least one medication was being administered to the patient.

In some embodiments, a data command center visual display system that displays data on a display includes a computing device comprising at least one processor, a non-transitory computer-readable medium, having stored thereon, software instructions that when executed by the at least one processor of the computing device, cause the computing device to perform operations including at least, linking to and receiving patient related medical records including patient data from at least one patient data source, wherein the patient data includes at least one of medical services, clinical data, examination findings, diagnostic tests, and procedures performed on one or more patients, determining, from at least one of the patient data and the at least one of the medical services, the clinical data, the examination findings, the diagnostic tests, and the procedures, prescribed or medications prescribed or administered to the one or more patients, generating a respective graphical representation for each of the determined medications prescribed or administered to the one or more patients, and displaying using the one or more windows, at least one of medical services, clinical data, examination findings, diagnostic tests, and procedures performed on one or more patients and at least one generated, respective graphical representation of at least one medication administered to a patient in context with at least one of the patient data and the at least one of the medical services, the clinical data, the examination findings, the diagnostic tests, and the procedures, wherein the at least one of the medical services, the clinical data, the examination findings, the diagnostic tests, and the procedures are arranged on the display according to at least one of a time and a date that the medical services, the clinical data, the examination findings, the diagnostic tests and the procedures were performed on the one or more patients, and wherein the at least one generated, respective graphical representation of the at least one medication administered to the patient is arranged on the display according to at least one of the times and the dates that the at least one medication was being administered to the patient.

In some embodiments, a method for a display of a graphical representation of complete medical history of a patient in a data command center comprising one or more windows for display and including patient-related data received or derived from at least one patient database, the method includes determining, from the patient-related data, a complete medical history of at least one patient including at least one of medical services, clinical data, examination findings, diagnostic tests, medications prescribed or administered to and procedures performed on a patient, as well as cancelled or missed visits, generating a graphical representation of the determined complete medical history of the patient including the at least one of medical services, clinical data, examination findings, diagnostic tests, identified or prescribed medications, and procedures performed on the patient, and displaying the generated graphical representation in the at least one or more windows according to at least one of a time and a date that the at least one of the medical services, the clinical data, the examination findings, the diagnostic tests, and the procedures the medical services, the clinical data, the examination findings, the diagnostic tests and the procedures were performed on the one or more patients and at least one of the times and the dates that the medications were being administered to the patient, wherein a user is enabled to select a location in the displayed graphical representation and details regarding the at least one of medical services, clinical data, examination findings, diagnostic tests, medications prescribed or administered to and procedures performed on the patient related to that selected location are presented to the user. In one embodiments, the system allows connecting to home monitoring devices, systems such as I phone watches that monitor constantly Blood pressure and pulse and can discover if patient may be having a heart attack and can display one of a time or date that no medical service was performed by the Doctor but a clinical measurement by the patient or outside entity can be displayed including time and date medications were actually taken by the patient or physical therapy was performed by the patient. In some embodiments this information can be displayed along with time and dates medical services were provided or can be selected to be separate. The system can monitor these times and dates that measurements by the patient or outside entity performed the clinical measurement for instance blood pressure, pulse, reading, blood sugar readings and when critical new data that exceeds a threshold occurs, alerts and expandable fields can be inserted within the data fields of the time and dates of medical services even if the user chooses an option not to comingle home monitoring for instance with measurement's during time and dates that a medical service occurs. In some embodiment cancelled or missed appointments and future appointments and any medical service or action to have been or to be performed are displayed so the user can identify the impact, necessity, and correctness of what was or is to be performed and which may have an impact on any date of service.

In some embodiments, multiple aspects of this invention may be displayed and correlated against each other, or groups of embodiments, or as a whole, such as representing summary groupings of results for specific disease states alongside graphical representations of relevant results, contributing factors, life events, medical procedures, medications, and all other data represented herein. Correlation may be automated in accordance with principles defined herein, and may employ clinical decision support in determining which aspects to display.

Other and further embodiments in accordance with the present principles are described below.

### DESCRIPTION OF THE DRAWINGS

FIG. 1 depicts a high-level block diagram of a Data Command Center in accordance with an embodiment of the present principles.

FIG. 2 depicts a high-level block diagram of a computing device 200 suitable for use with embodiments of a Data Command Center in accordance with the present principles.

FIG. 3 depicts a high-level infrastructure diagram of a Data Command Center in accordance with an embodiment of the present principles.

FIG. 4 illustrates a logical implementation of a multitenant example of Dynamic Health Records with a Co-Management Connection to allow for sharing of data.

FIG. 5 illustrates a high-level data knowledge storage system in accordance with an embodiment of the present principles.

FIG. 6 illustrates a high-level data categorization system in accordance with an embodiment of the present principles.

FIG. 7 illustrates a high-level data category search system in accordance with an embodiment of the present principles.

FIG. 8 illustrates a high-level data correlation system in accordance with an embodiment of the present principles.

FIG. 9 Illustrates a standard workflow when data is received from an external system through any of the messaging standards or API methods in accordance with the tool described herein.

FIG. 10 illustrates examples of different data being accessed.

FIG. 11 depicts a flow diagram of a method for Unique Patient Identification in a Data Command Center in accordance with an embodiment of the present principles.

FIG. 12 illustrates an exemplary clinical decision support system in accordance with the tool described herein.

FIG. 13A depicts a first portion of a workflow diagram of a process for intelligently expanding, collapsing, highlighting, emphasizing, graying, displaying, and/or hiding columns, rows and/or any other portion of the medical records dashboard in accordance with an embodiment of the present principles.

FIG. 13B depicts a second portion of the workflow diagram of a process of FIG. 48A for intelligently expanding, collapsing, highlighting, graying, displaying, and/or hiding columns, rows and/or any other portion of the medical records dashboard in accordance with an embodiment of the present principles.

FIG. 13C depicts a third portion of the workflow diagram of a process of FIG. 48A for intelligently expanding, collapsing, highlighting, graying, displaying, and/or hiding columns, rows and/or any other portion of the medical records dashboard in accordance with an embodiment of the present principles.

FIG. 13D depicts a flow diagram of a method for rules-based data display in a data command center comprising a medical records dashboard in accordance with an embodiment of the present principles.

FIG. 14 illustrates examples of different ways data fields may be dynamically updated in accordance with an embodiment of the present principles.

FIG. 15 depicts a series of data points associated with the determination of how and when to display data fields.

FIG. 16A depicts a first portion of configuration options associated with dynamic data representation.

FIG. 16B depicts a second portion of configuration options associated with dynamic data representation.

FIG. 17 depicts a trigger processing module in accordance with principals denoted in FIGS. 16A and 16B.

FIG. 18 illustrates an example of a critical patient indicator.

FIG. 19 illustrates a series of configurations in accordance with an embodiment of the present principles.

FIG. 20 illustrates interactions between data sets in accordance with an embodiment of the present principles.

FIG. 21 illustrates a weighting system in accordance with an embodiment of the present principles.

FIG. 22 illustrates a weighting system flow diagram in accordance with an embodiment of the present principles.

FIG. 23 illustrates dynamic, interactive header and summary rows in accordance with an embodiment of the present principles.

FIG. 24A further illustrates a first portion of dynamic, interactive header and summary rows in accordance with an embodiment of the present principles.

FIG. 24B further illustrates a second portion of dynamic, interactive header and summary rows in accordance with an embodiment of the present principles.

FIG. 25A depicts a first portion of a patient specific dashboard in accordance with an embodiment of present principals.

FIG. 25B depicts a second portion of a patient specific dashboard in accordance with an embodiment of present principals.

FIG. 26A depicts a first portion of additional data display within a patient specific dashboard in accordance with an embodiment of present principals.

FIG. 26B depicts a second portion of additional data display within a patient specific dashboard in accordance with an embodiment of present principals.

FIG. 27A depicts a first portion of an ordering module within a patient specific dashboard in accordance with an embodiment of present principals.

FIG. 27B depicts a second portion of an ordering module within a patient specific dashboard in accordance with an embodiment of present principals.

FIG. 28A depicts a first portion of a future order and filtering within patient specific dashboard in accordance with an embodiment of present principals.

FIG. 28B depicts a second portion of a future order and filtering within patient specific dashboard in accordance with an embodiment of present principals.

FIG. 29A depicts a first portion of in-context alert configuration within a patient specific dashboard in accordance with an embodiment of present principals.

FIG. 29B depicts a second portion of in-context alert configuration within a patient specific dashboard in accordance with an embodiment of present principals.

FIG. 30A depicts a first portion of an embodiment of a medical records dashboard in accordance with another embodiment of the present principles.

FIG. 30B depicts a second portion of an embodiment of a medical records dashboard in accordance with another embodiment of the present principles.

FIG. 30C depicts a third portion of an embodiment of a medical records dashboard in accordance with another embodiment of the present principles.

FIG. 30D depicts a fourth portion of an embodiment of a medical records dashboard in accordance with another embodiment of the present principles.

FIG. 30E depicts a fifth portion of an embodiment of a medical records dashboard in accordance with another embodiment of the present principles.

FIG. 30F depicts a sixth portion of an embodiment of a medical records dashboard in accordance with another embodiment of the present principles.

FIG. 30G depicts a seventh portion of an embodiment of a medical records dashboard in accordance with another embodiment of the present principles.

FIG. 30H depicts an eighth portion of an embodiment of a medical records dashboard in accordance with another embodiment of the present principles.

FIG. 30I depicts a ninth portion of an embodiment of a medical records dashboard in accordance with another embodiment of the present principles.

FIG. 31 illustrates a horizontal correlative graph in accordance with an embodiment of the present principles.

FIG. 32A depicts a first portion of a correlative line graph in accordance with an embodiment of present principals.

FIG. 32B depicts a second portion of a correlative line graph in accordance with an embodiment of present principals.

FIG. 33A depicts a first portion of an augmented correlative line graph in accordance with an embodiment of present principals.

FIG. 33B depicts a second portion of an augmented correlative line graph in accordance with an embodiment of present principals.

FIG. 34A illustrates a first portion of direct access and parameter setting on a horizontal graph in accordance with an embodiment of the present principles.

FIG. 34B illustrates a second portion of direct access and parameter setting on a horizontal graph in accordance with an embodiment of the present principles.

FIG. 35A depicts a first portion of three different representations of an intelligent alert configuration system overlayed upon several different aspects of an application in accordance with an embodiment of the present principles.

FIG. 35B depicts a second portion of three different representations of an intelligent alert configuration system overlayed upon several different aspects of an application in accordance with an embodiment of the present principles.

FIG. 36 illustrates a whole life view zoomed out in accordance with an embodiment of the present principles.

FIG. 37 illustrates a whole life view zoomed in to a first level in accordance with an embodiment of the present principles.

FIG. 38 illustrates a whole life view fully zoomed in in accordance with an embodiment of the present principles.

FIG. 39A depicts a first portion of a graphical view of the entire medical history of a patient as a Whole Life tool in accordance with an embodiment of the present principles.

FIG. 39B depicts a second portion of a graphical view of the entire medical history of a patient as a Whole Life tool in accordance with an embodiment of the present principles.

FIG. 39C depicts a third portion of a graphical view of the entire medical history of a patient as a Whole Life tool in accordance with an embodiment of the present principles.

FIG. 40 depicts multiple embodiments of this invention correlated with each other.

FIG. 41 illustrates a logical implementation of a multitenant example of Dynamic Health Records with an External Data Access Point Connection to allow for sharing of data.

FIG. 42 depicts an embodiment of a shared configuration interface.

FIG. 43 depicts a flow diagram of a method for shared in accordance with an embodiment of the present principles.

FIG. 44 depicts an embodiment of a co-managed medical records dashboard in the Data Command Center of the present principles in accordance with one embodiment.

FIGS. 45A and 45B depict a medical records dashboard including an ability to launch a Co-Management process in accordance with an embodiment the present principles.

FIGS. 46A and 46B depict a medical records dashboard including a custom template creation process for co-management in accordance with an embodiment of the present principles.

FIG. 47A depicts a first portion of a workflow diagram of a Co Management process in accordance with an embodiment of the present principles.

FIG. 47B depicts a second portion of the workflow diagram of the Co Management process of FIG. 47A in accordance with an embodiment of the present principles.

FIG. 48 depicts a flow diagram of a method for Co-Management of patient information in a medical records dashboard in accordance with an embodiment of the present principles.

FIG. 49 depicts a shared dashboard configuration in accordance with an embodiment of the present principles.

FIG. 50 depicts healthcare roles and systems utilized by each role.

FIG. 51 depicts data associated with healthcare roles.

FIG. 52 depicts a centralized Data Command Center Dashboard in accordance with an embodiment of the present principles.

The figures are not drawn to scale and may be simplified for clarity. It is contemplated that elements and features of one embodiment may be beneficially incorporated in other embodiments without further recitation.

### DETAILED DESCRIPTION

Embodiments of the present principles generally relate to a Data Command Center, also referred to as dynamic health record system or dynamic health record for displaying data on a display screen from multiple data sources and enabling navigation amongst the data on a single display. While the concepts of the present principles are susceptible to various modifications and alternative forms, specific embodiments thereof are shown by way of example in the drawings and are described in detail below. It should be understood that there is no intent to limit the concepts of the present principles to the particular forms disclosed. On the contrary, the intent is to cover all modifications, equivalents, and alternatives consistent with the present principles and the appended claims. For example, although embodiments of the present principles will be described primarily with respect to inter-function with an EMR system, such teachings should not be considered limiting. Embodiments in accordance with the present principles can inter-function with other informational systems such as Health Information Exchanges (HIEs), Billing Clearinghouses, Insurance Companies, Picture Archiving and Communication Systems (PACS) as well as third party services and the like.

In addition, the tool embodiments of the present principles are not limited in application to the details of construction and the arrangement of components set forth in the following description or illustrated in the following drawings. Embodiments of the present principles are capable of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “connected,” “supported,” and “coupled” and variations thereof are used broadly and encompass both direct and indirect mountings, connections, supports, and couplings. Further, “connected” and “coupled” are not restricted to physical or mechanical connections or couplings.

As used herein, the term “medical care provider” is intended to represent any healthcare provider/clinical professional such as a doctor, physician, podiatrist, chiropractor, dentist, veterinarian, ancillary staff, nurses, physician's assistant, medical care provider, physical therapist, all allied health professionals, and/or hospital staff member. All such healthcare providers/clinical professional can implement embodiments of the present principles the tool as interchangeable users.

As used herein a row, column, or line of items (even a diagonal line) is intended to represent a sequencing or evaluation of information in any direction. In the embodiments depicted herein, information does not have to be depicted as having a visual or physical separation in the vertical or horizontal direction to be defined as being a row or column. In accordance with the present principles, items next to each other horizontally, lined up in such a way that straight lines above and below can be drawn, and items fall between those two horizontal lines, can be considered as being in a row. Items in rows can be related by similar time or other common or same denominator, such as a medical service, procedure, image, or financial number, so that a user can quickly visualize trends or changes in those items. Similarly, items next to each other vertically, lined up in such a way that straight lines to the left and to the right can be drawn can be considered as being in a column. In some embodiments, items can be arranged diagonally and be considered to be in a row or a column.

As used herein, Practice Management Systems (PMs) are programs that perform the billing collection and reconciliation of payments as well as scheduling patients. PMs can also be referred to as Revenue Cycle Management (RCM) and have associated billing companies that use software to help practices and medical care providers get the bills out and collect money from insurance companies. In some embodiment, these entities can integrate with and work through clearing houses.

In the embodiments described herein, the terms window screen, scrolling screen, display, view, snapshot, and the like can be used interchangeably and are intended to represent a single instance of the presentation of medical information associated with at least one patient. In the described embodiments, the single instance can be presented on one or more windows, in a single or multiple screens, a scrolling screen, in one or more views and using one or more snapshots. For example, in some embodiments in accordance with the present principles a user can access different panels from a scrolling screen and converge the panels into a single view or snapshot. That is, in accordance with the present principles, a user is able to compile data/information from various windows, screens, scrolling screens, displays, snapshots and the like and create a single instance presentation including the data/information of interest to the user for at least one patient. In accordance with the present principles, a single instance presentation can be presented on more than one monitor at a time. As used herein, the term single instance presentation is intended to describe a single display interface that is not limited to a single monitor. That is, in some embodiments, what defines a single instance presentation is the fact that there is a single interface, a single control that controls the presentation of the date/information, which can be then be viewed on one or more monitors or other means.

The term medical tests as described herein is intended to describe medical procedures performed for or on patients including but not limited to image or imaging, diagnostic tests, radiological tests or procedures, laboratories, chemistry and hematological tests, photography, genetic testing, nuclear scans, ultrasounds, x-rays, optical coherent tomography photographs and angiographies, assessments and plans, letters, examination findings and any medical testing or medical services that tests or screens patients for a medical condition, which in some instance can be identified by CPT codes. It should be further noted that in some instances, terms like diagnosis can be reflected by ICD 9 or 10 or similar identifying factors, and medications can be interchangeable.

As used herein, the terms icon, symbol, and indicator are all interchangeable and are intended to describe a visual element enabling the access of additional underlying information and having the ability to convey additional information simply by their presentation. That is, such visual elements can convey information by their display which can include such visual presentations including but not limited to words, numbers, blinking elements, flashing elements, color changing elements, elements in italics, underlined elements, and the like or any means that draws the attention of a user.

The reference to a medical records dashboard of the present principles described throughout the teachings herein is intended to refer to any embodiment of a medical records dashboard according to the present principles that is applicable to a currently described embodiment.

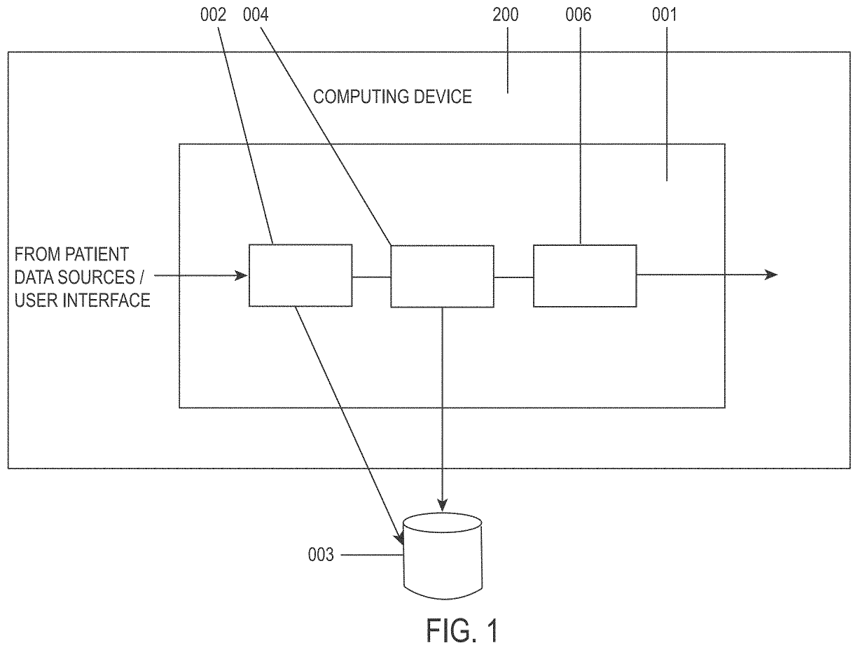

FIG. 1 depicts a high-level block diagram of a Data Command Center (DCC) 001 in accordance with an embodiment of the present principles. In the embodiment of FIG. 1, the Data Command Center 001 illustratively comprises an integration module 002 (i.e., to interface data between an EMR and the DCC), a Rules module 004 (i.e., to determine where and how the data is to be displayed), and a display module 006 (i.e., to display the data in the appropriate place). In the embodiment of FIG. 1, the integration module 002 and the rules module 004 can be in communication with a data storage 003. For example, the integration module 002 can store data from patient data sources in the data storage 003 and the rules module 004 can access the data storage 003 to retrieve data and/or information stored therein.

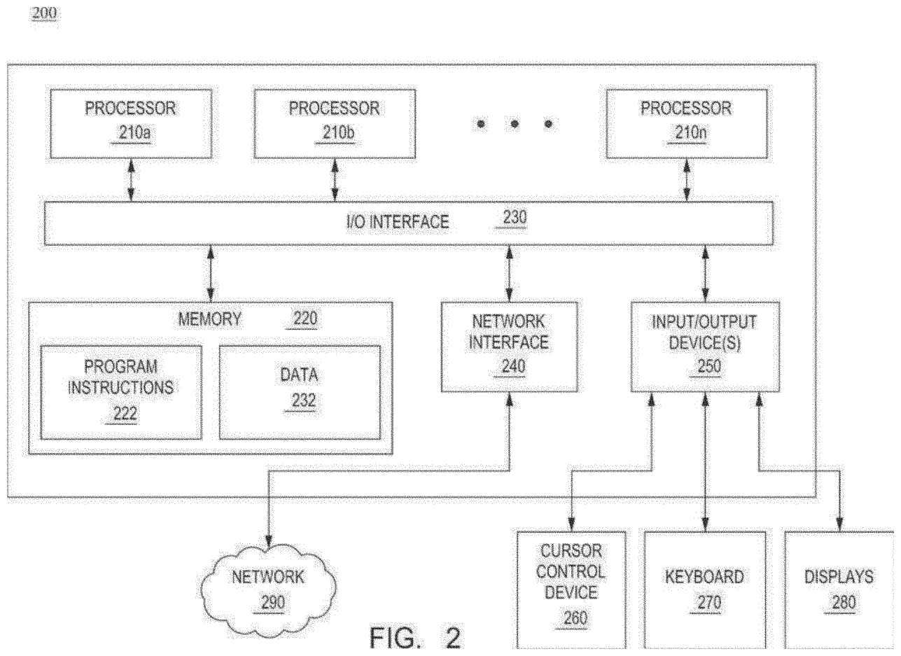

As depicted in FIG. 1, embodiments of a Data Command Center in accordance with the present principles, such as the Data Command Center 001 of FIG. 1, can be implemented in a computing device 200. FIG. 2 depicts a high-level block diagram of a computing device 200 suitable for use with embodiments of a Data Command Center in accordance with the present principles such as the user Data Command center 001 of FIG. 1. In some embodiments, the computing device 200 can be configured to implement methods of the present as processor-executable executable program instructions 222 (e.g., program instructions executable by processor(s) 210) in various embodiments.

In the embodiment of FIG. 2, the computing device 200 includes one or more processors 210a-210n coupled to a system memory 220 via an input/output (I/O) interface 230. The computing device 200 further includes a network interface 240 coupled to I/O interface 230, and one or more input/output devices 250, such as cursor control device 260, keyboard 270, and display(s) 280. In various embodiments, a user interface can be generated and displayed on display 280. In some cases, it is contemplated that embodiments can be implemented using a single instance of computing device 200, while in other embodiments multiple such systems, or multiple nodes making up the computing device 200, can be configured to host different portions or instances of various embodiments. For example, in one embodiment some elements can be implemented via one or more nodes of the computing device 200 that are distinct from those nodes implementing other elements. In another example, multiple nodes may implement the computing device 200 in a distributed manner.

In different embodiments, the computing device 200 can be any of various types of devices, including, but not limited to, a personal computer system, desktop computer, laptop, notebook, tablet or netbook computer, mainframe computer system, handheld computer, workstation, network computer, a camera, a set top box, a mobile device, a consumer device, video game console, handheld video game device, application server, storage device, a peripheral device such as a switch, modem, router, or in general any type of computing or electronic device.

In various embodiments, the computing device 200 can be a uniprocessor system including one processor 210, or a multiprocessor system including several processors 210 (e.g., two, four, eight, or another suitable number). Processors 210 can be any suitable processor capable of executing instructions. For example, in various embodiments processors 210 may be general-purpose or embedded processors implementing any of a variety of instruction set architectures (ISAs). In multiprocessor systems, each of processors 210 may commonly, but not necessarily, implement the same ISA.

System memory 220 can be configured to store program instructions 222 and/or data 232 accessible by processor 210. In various embodiments, system memory 220 can be implemented using any suitable memory technology, such as static random-access memory (SRAM), synchronous dynamic RAM (SDRAM), nonvolatile/Flash-type memory, or any other type of memory. In the illustrated embodiment, program instructions and data implementing any of the elements of the embodiments described above can be stored within system memory 220. In other embodiments, program instructions and/or data can be received, sent, or stored upon different types of computer-accessible media or on similar media separate from system memory 220 or computing device 200.

In one embodiment, I/O interface 230 can be configured to coordinate I/O traffic between processor 210, system memory 220, and any peripheral devices in the device, including network interface 240 or other peripheral interfaces, such as input/output devices 250. In some embodiments, I/O interface 230 can perform any necessary protocol, timing, or other data transformations to convert data signals from one component (e.g., system memory 220) into a format suitable for use by another component (e.g., processor 210). In some embodiments, I/O interface 230 can include support for devices attached through various types of peripheral buses, such as a variant of the Peripheral Component Interconnect (PCI) bus standard or the Universal Serial Bus (USB) standard, for example. In some embodiments, the function of I/O interface 230 can be split into two or more separate components, such as a north bridge and a south bridge, for example. Also, in some embodiments some or all of the functionality of I/O interface 230, such as an interface to system memory 220, can be incorporated directly into processor 210.

Network interface 240 can be configured to allow data to be exchanged between the computing device 200 and other devices attached to a network (e.g., network 290), such as one or more external systems or between nodes of the computing device 200. In various embodiments, network 290 can include one or more networks including but not limited to Local Area Networks (LANs) (e.g., an Ethernet or corporate network), Wide Area Networks (WANs) (e.g., the Internet), wireless data networks, some other electronic data network, or some combination thereof. In various embodiments, network interface 240 can support communication via wired or wireless general data networks, such as any suitable type of Ethernet network, for example, via digital fiber communications networks; via storage area networks such as Fiber Channel SANs, or via any other suitable type of network and/or protocol.

Input/output devices 250 can, in some embodiments, include one or more display terminals, keyboards, keypads, touchpads, scanning devices, voice or optical recognition devices, or any other devices suitable for entering or accessing data by one or more computer systems. Multiple input/output devices 250 can be present in computer system or can be distributed on various nodes of the computing device 200. In some embodiments, similar input/output devices can be separate from the computing device 200 and can interact with one or more nodes of the computing device 200 through a wired or wireless connection, such as over network interface 240.

Those skilled in the art will appreciate that the computing device 200 is merely illustrative and is not intended to limit the scope of embodiments. In particular, the computer system and devices can include any combination of hardware or software that can perform the indicated functions of various embodiments, including computers, network devices, Internet appliances, PDAs, wireless phones, pagers, and the like. The computing device 200 can also be connected to other devices that are not illustrated, or instead can operate as a stand-alone system. In addition, the functionality provided by the illustrated components can in some embodiments be combined in fewer components or distributed in additional components. Similarly, in some embodiments, the functionality of some of the illustrated components may not be provided and/or other additional functionality can be available.

The computing device 200 can communicate with other computing devices based on various computer communication protocols such a Wi-Fi, Bluetooth®. (and/or other standards for exchanging data over short distances includes protocols using short-wavelength radio transmissions). USB, Ethernet, cellular, an ultrasonic local area communication protocol, etc. The computing device 200 can further include a web browser.

Although the computing device 200 is depicted as a general purpose computer, the computing device 200 is programmed to perform various specialized control functions and is configured to act as a specialized, specific computer in accordance with the present principles, and embodiments can be implemented in hardware, for example, as an application specified integrated circuit (ASIC). As such, the process steps described herein are intended to be broadly interpreted as being equivalently performed by software, hardware, or a combination thereof.

Those skilled in the art will also appreciate that, while various items are illustrated as being stored in memory or on storage while being used, these items or portions of them can be transferred between memory and other storage devices for purposes of memory management and data integrity. Alternatively, in other embodiments some or all of the software components can execute in memory on another device and communicate with the illustrated computer system via inter-computer communication. Some or all of the system components or data structures can also be stored (e.g., as instructions or structured data) on a computer-accessible medium or a portable article to be read by an appropriate drive, various examples of which are described above. In some embodiments, instructions stored on a computer-accessible medium separate from the computing device 200 can be transmitted to the computing device 200 via transmission media or signals such as electrical, electromagnetic, or digital signals, conveyed via a communication medium such as a network and/or a wireless link. Various embodiments can further include receiving, sending, or storing instructions and/or data implemented in accordance with the foregoing description upon a computer-accessible medium or via a communication medium such as cloud based storage. In general, a computer-accessible medium can include a storage medium or memory medium such as magnetic or optical media, e.g., disk or DVD/CD-ROM, volatile or nonvolatile media such as RAM (e.g., SDRAM, DDR. RDRAM, SRAM, and the like), ROM, and the like.

When a patient record is shared with another medical professional, if the professional does not have access to the Data Command Center of the present principles, the other medical professional can receive an email to register for access to the Data Command Center. In some embodiments, if the professional does have an account but a new patient is being shared, the physician can receive an email notification. The new external user will only have access to the specific patients that are shared. Such sharing of patient medical records amongst the patient's physicians better enables the physicians to work together to follow preferred practice patterns for patient treatment as may be required by insurance companies and/or the government. This process is particularly helpful for managing patients with certain chronic diseases like diabetes in which a nephrologist, podiatrist, ophthalmologist, endocrinologist, and family physician need to see each other's results. Another example is shared care before and after cataract surgery where optometrists and ophthalmologist need to see each other's results.

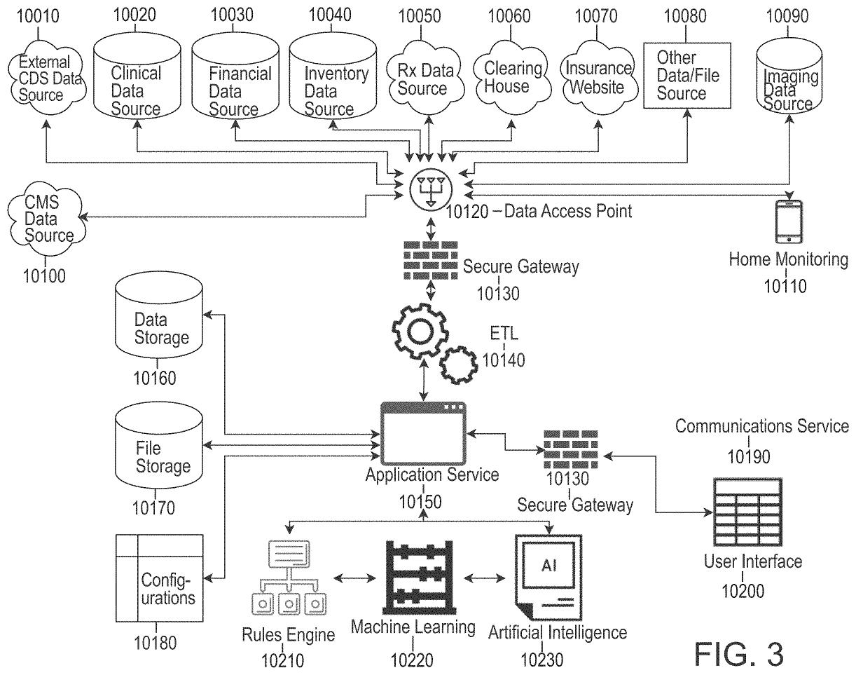

FIG. 3 depicts an embodiment of a Data Command Center architecture in accordance with an alternate embodiment of the present principles. FIG. 3 further depicts how the Data Command Center connects to external Health Information Technology systems and third party services 10010-10110. The Data Command Center architecture of FIG. 3 is a multi-tenant cloud-based web application. Multi-tenant infers that the application is deployed once, and all customers access the same server or farm. In the embodiment of FIG. 3, data is segregated by the application so that customers can only access their own data. The Data Command Center is accessible over the Internet via a user interface 10200 through a Secure Gateway 10130.

The Data Command Center architecture illustrated in FIG. 3 is one embodiment of how the system can be configured to access data from multiple sources, compile and evaluate the data, take action, and display the data to the end user. In FIG. 3, for security purposes, access to the cloud platform containing the Data Command Center is governed by a Secure Gateway 10130, whether a connection exists between the Data Command Center and the end user or the Data Command Center and external data sources. Additionally, end to end encryption can be an inherent part of the Data Command Center architecture of FIG. 3 as the Data Command Center architecture is optimized to meet the highest privacy and security expectations. Each component allows for both the application of current high strength encryption (ex. AES 256) as well as continuous implementation of evolving data protection and security best practices.

In the embodiment of FIG. 3, the exchange of information between multiple external health information technology (HIT) systems 10010-10110 and the Application Service 10150 is routed through an external Data Access Point 10120. Exchange of data may occur in a monodirectional or bidirectional manner, dependent upon access and scenario. In such embodiments, external data sources can include, but are not limited to:

External CDS Data Sources: External Clinical Decision Support Data Sources 10010 may include Registries, Societies, Industry Resources, Insurance Companies, and other sources make available relevant rules and data to evaluate against.

Clinical Data Sources: Clinical Data Sources 10020 may include EHR/EHRs, available anonymized Clinical Data Sources, Referral Management Data Sources, and other available clinical data repositories. Clinical data may be read and/or written back to the data source.

Financial Data Sources: Financial Data Sources 10030 may include banks, Clearing Houses, Insurance Data Sources, Center for Medicare and Medicaid Services Data Sources, and other repositories of financial data. Financial Data may be read and/or written back to the data source.

Inventory Data Sources: Inventory Data Sources 10040 may include internal and/or external Inventory Management Systems. Inventory Data may be read and/or written back to the data source.

Rx Data Sources: Rx Data Sources 10050 may include available e-Prescribing Data Sources, Pharmacy Data Sources, and other external Prescription Data Sources. Rx Data may be read and/or written back to the data source.

Clearing House: Clearing Houses 10060 contain large amounts of Financial and Insurance Data, access to Insurance Data Sources, and Claims Scrubbing Mechanisms. Clearing House data may be read and/or written back to the data source.

Insurance Websites: Insurance Websites 10070 offer direct interaction with Insurance Data Sources beyond standardized Clearing House access. Insurance Website data may be read and/or written back to the data source.

Other Data/File Sources: A key benefit of the Command Center is the ability to pull relevant data from various, disparate data sources. Other Data/File Sources 10080 may include any repository of relevant data, Clinical, Insurance, Financial, CDS, or otherwise. Other data sources may comprise non-standard data repositories, smartphone apps, or even a Google or Excel spreadsheet that a practice records relevant data in. Other Data/File Sources may be read and/or written back to the data source.

Imaging Data Source: Imaging Data Sources 10090 may consist of locally hosted image repositories, diagnostic testing systems, cloud-based image repositories, or other sources of medically relevant imaging.

CMS Data Source: The Center for Medicare and Medicaid Services (CMS) 10100 make available patient data for Medicare and Medicaid patients for research and development.

Home Monitoring Devices: With the advent of the IoT, more and more home-based monitoring devices 10110 are being used to track important health information from a patient's home. Such information may be relevant to patient care, and as such, the Command Center reads, compiles, and evaluates said data.

In the embodiment of the Data Command Center of FIG. 3, data can be posted and/or retrieved from External Data Sources 10010-10110, routed through a Data Access Point 10120, validated by a Secure Gateway 10130, and then may be processed through an ETL (Extract Transform Load) service 10140. The ETL service 10140 is utilized by the Data Command Center to perform necessary transformation of data from proprietary or non-standard formats into industry standard formats that are universal, manageable, and portable. It should be appreciated that an inverse ETL process can be utilized to take previously transformed data and return said data in its initial format.

Utilizing such a technique, no one data source need be solely responsible for any given data point. Where accessible, data which can be missing from one system can be imported from a different system if such data exists. Employing the same logic, missing data which may not be found elsewhere can be flagged as missing to inform a user such data is not present in any relevant data source.

A service or group of services, referred to as the Application Service 10150, manage the routing of data between storage 10160-10170, rules engine 10210-10230 services, configuration services 10180, and the user interface 10200 and/or communications service 10190. The Application Service 10150 is responsible for the overall management of data and other services. Data, after transformed into industry standard formats, can be stored in a Data Storage repository 10160, in one or multiple formats dependent upon use case. Data may also not be stored, and directly posted to the User Interface 10200 through a Secure Gateway 10130. Data can also be converted to files and stored within the File Storage repository 10170. Files received can be stored in the File Storage repository 10170, can be reformatted into industry standard formats by the ETL service 10140 and stored in the File Storage repository 10170, or can be transformed into data and stored in the Data Storage repository 10160. Files may also not be stored and directed posted to the User Interface 10200 through a Secure Gateway 10130. Data and Files, stored or not stored, can be posted to the User Interface 10200 through a Secure Gateway 10130 with additional information and/or edits/enhancements/augmentation to their content.

Data and Files can, dependent upon use case, be processed through a Rules Engine 10210 responsible for evaluating a set of rules against the Data and Files. Rules can be predefined, retrieved from external data sources 10010-10110, generated at runtime, and/or generated based on Machine Learning 10220 and/or Artificial Intelligence 10230. Machine Learning 10220 can employ a number of techniques, to adapt to new information, and Artificial Intelligence 10230 may compile, coordinate, and evaluate data for trends to further define new rules.

All relevant data can then be processed through the Application Service 10150 and can be returned to the User Interface 10200 through a Secure Gateway 10130, ensuring proper security and encryption is in place to protect sensitive information. The Configurations service 10180 can store predefined lists used to determine which data can be displayed and can work alone or in conjunction with the Rules services 10210-10230 to make the determinations. The Rules services 10210-10230 can also utilize the Application Service 10150 to access the Communications service 10190 for external automated communications, where appropriate. It should be appreciated that best practices, and evolving technology, can be used to determine the best approach for data retrieval, transformation, storage, and transmittal in accordance with the present principles.

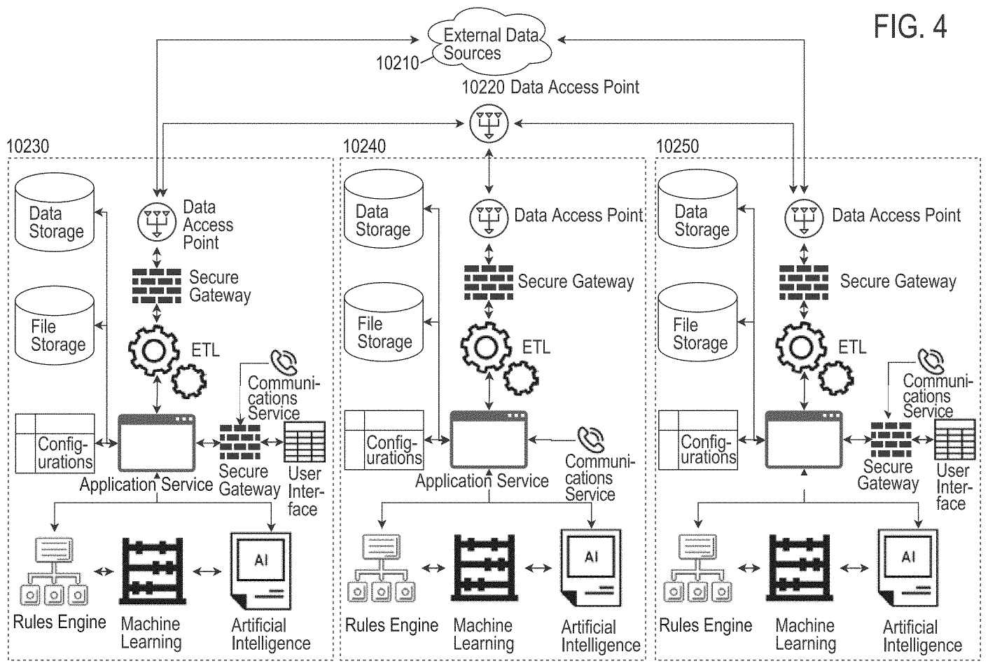

FIG. 4 depicts an implementation diagram of the ability of the Data Command Center of the present principles to connect several instances of the application to each other, as well as to external data sources, such that all can function as a single, logical application. As depicted in FIG. 4, utilizing a multi-tenant architecture, all data can be centralized, and managed by the Application service 10150 of, for example, FIG. 3. Logical separation of data occurs at the software level. That is, FIG. 4 is an example of an implementation diagram whereby multiple tenants 10230, 10240 can subscribe to multiple external data sources 10210, which can consist of both unique and shared resources. In this example, the first Client 10230 and second Client 10240 can exist within a single server or farm, yet the logical separation of data still exists. A third instance of the application 10240 can be used to connect the Clients to each other through use of an external or internal Data Access Point 10220. As such, each Client functions as its own entity, yet sharing of data between clients can be achieved.

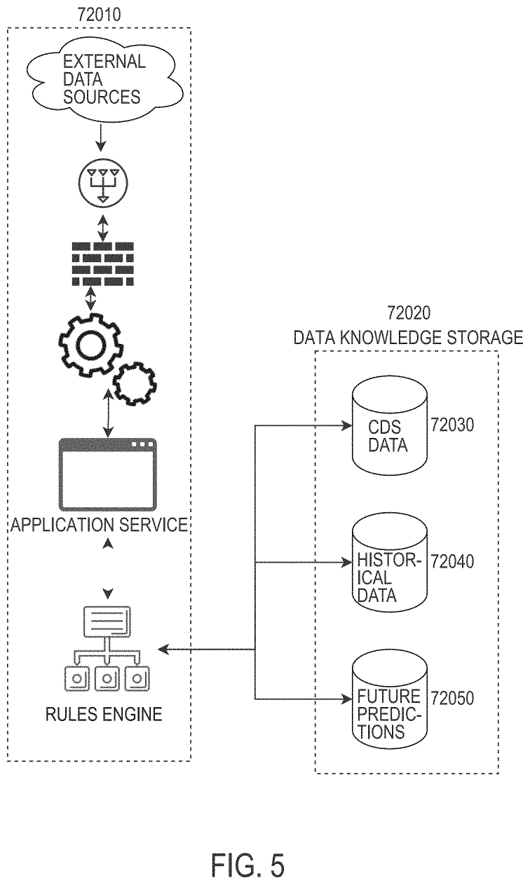

FIG. 5 illustrates a high-level data knowledge storage system in accordance with an embodiment of the present principles. The process for interaction between external data sources 10010-10110 of FIG. 3 and the Rules Engine 10210 of FIG. 3 is summarized in 72010 of FIG. 5. As such, interaction with all available data is accessible to the Rules Engine. Subsequently, the Rules Engine can implement various storage mechanisms to maintain an accurate account of transformations and states of data. This can be achieved using, as shown in the example, Data Knowledge Storage 72020. Data Knowledge Storage can consist of separate repositories, or a single repository logically divided. Such data can be categorized, as in this example, as Clinical Decision Support data 72030, Historical Data 72040, and Future Predictions 72050. Clinical Decision Support data can be comprised of data directly accessed from External CDS Data Sources 10010 of FIG. 3, or any of the other relevant data sources, and can also be derived or compiled by the application, itself. Historical Data 72040 maintains an active record of augmentation and alteration performed by the Command Center to allow for the application to reference prior states of data at any given time. Future Prediction Data 72050 maintains an active record of predictions made for validation as well as to maintain a record of options available to correlate with actions other than those predicted for.

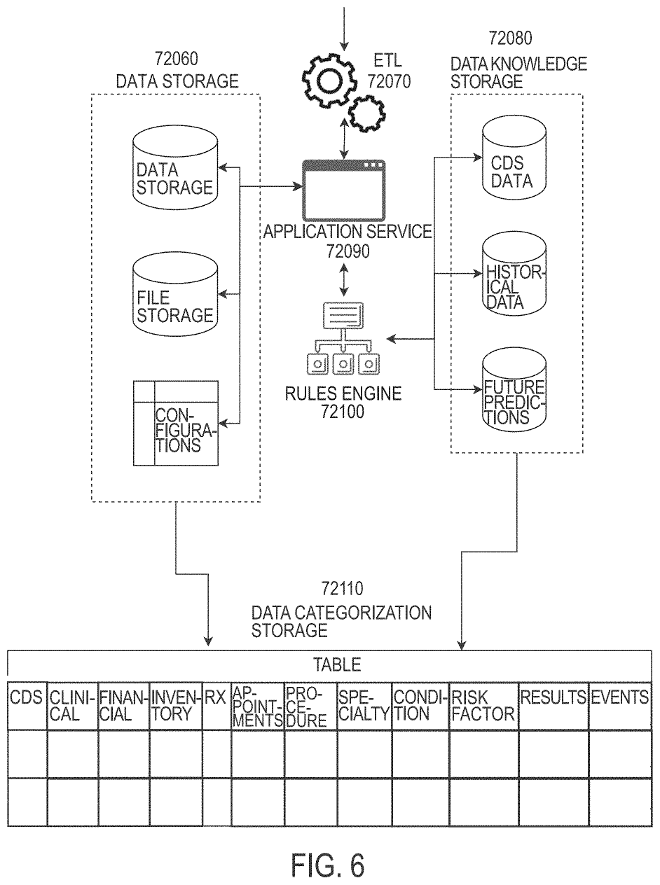

FIG. 6 illustrates a high-level data categorization system in accordance with an embodiment of the present principles. As previously described, data received from External Data Sources 10010-10110 of FIG. 3 is processed through an ETL process. Illustrated in FIG. 6 as 72070, this ETL process converts data received to standardized formats, which enables data of a similar category to be received from multiple sources. External data received is generally stored in the Data Storage 72060. Such data storage is managed through the Application Service 72090. Data generated by the application is generally stored in the Data Knowledge Storage 72080. Such data storage is managed through the Rules Engine 72100. All data storage is subsequently categorized for easy searchability and reporting purposes in the Data Categorization Storage 72110. Data is categorized into topics derived by requirements described herein. It should be noted that data is not limited to existing in a single category, and all data associated with a category can be stored or associated with its respective category.

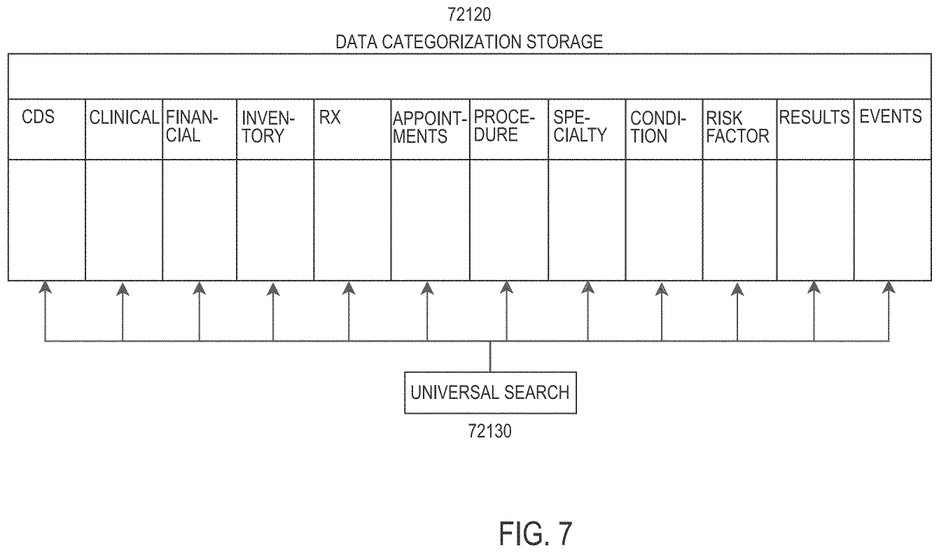

FIG. 7 illustrates a high-level data category search system in accordance with an embodiment of the present principles. In embodiments of the Data Command Center of the present principles, search boxes appear to query relevant data. Each search box can be individually configured to return data only related to a specific section of the application, but all search boxes can conform to a standardized Universal Search, as illustrated in FIG. 7. As data is received, transformed, and categorized, it is this final result of categorization by which a search is performed. The categorized information can reside in data storage such as Data Categorization Storage 72120, in a search-friendly format such as a star schema. The Universal Search box 72130 queries against this pre-categorized data, granting the ability to search data that may have originated from a variety of data sources through a single, universal search mechanism.

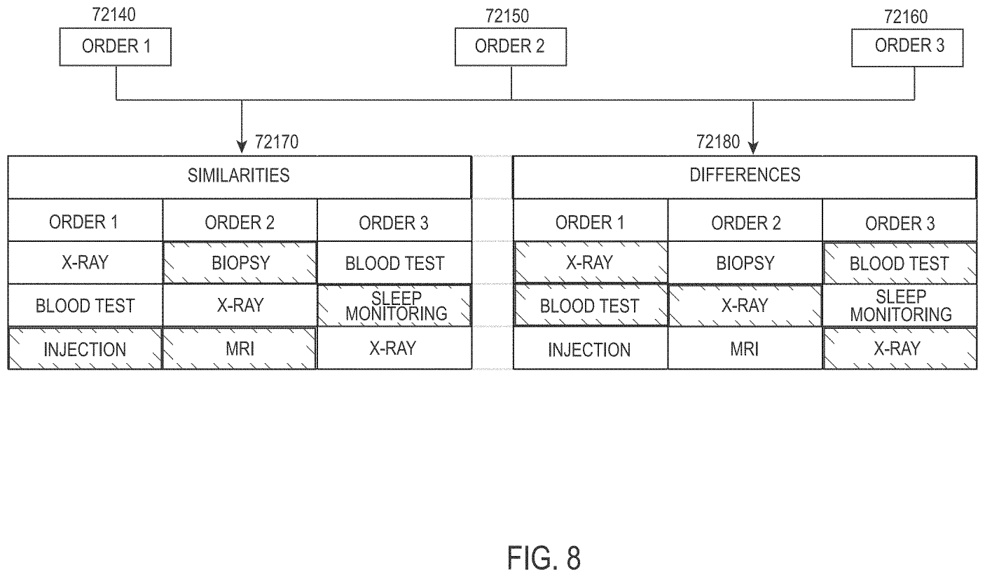

FIG. 8 illustrates a high-level data correlation system in accordance with an embodiment of the present principles. In embodiments of the present principles, as data is received from multiple sources, and collated from multiple practices, overlap becomes apparent. One doctor in one office may be attempting to schedule the same test or treatment as another doctor in another office. Or, perhaps, one doctor is scheduling a test or treatment that another has already completed. Such instances lead to wasted time and effort, while increasing cost. As such, when an event occurs, the Command Center searches all relevant data in the category to see if there is an existing record which would suffice the requirement. In the embodiment of FIG. 8, three orders 72140-72160 are being placed. The Data Command Center of the present principles reviews the orders for Similarities 72170 and Differences 72180. Identified Similarities, in this example, include X-Rays and Blood Tests. It is possible the same X-Ray is required by all requestors, and as such, the Data Command Center can suggest a single order to satisfy all requirements. It is also possible that each Blood Test requires the same or slightly different requirements. The Data Command Center can suggest multiple tests be run on a single sample to reduce redundancy. In the case of the differences, 72180, the Biopsy, Sleep Monitoring, Injection, and MRI are unique. The Data Command Center can still suggest utilization of a single resource, such as one lab, for all diagnostics.

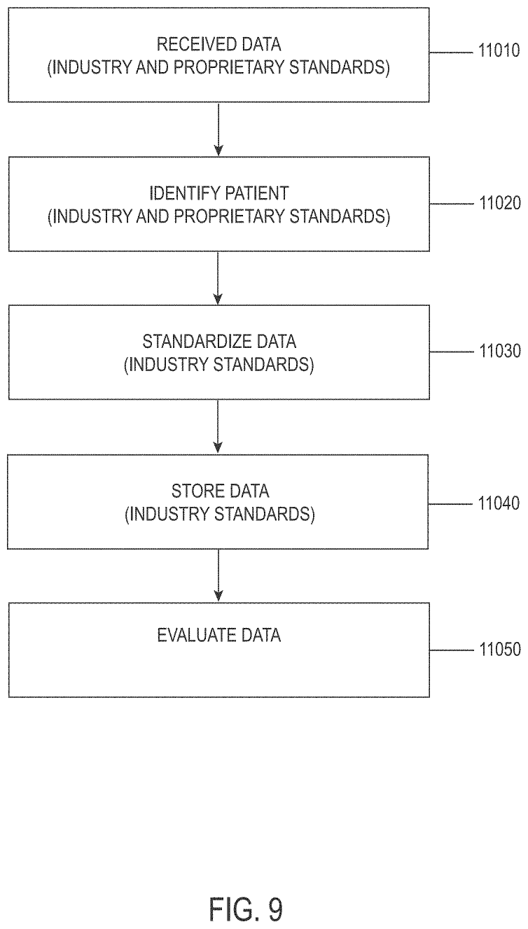

FIG. 9 depicts a high-level workflow diagram of the receipt, storage, and evaluation of data associated with a Data Command Center of the present principles. In the embodiment of FIG. 9, the Data Command Center can use constant poling at interval or monitoring for change to determine when new data is available. When data is received from an external system 10010-10100 through any of the messaging standards or API methods, a standard workflow can be followed as depicted in the embodiment of FIG. 9. The first step (11010) is to receive data using the previously discussed methods. The message is parsed and the patient identifying information is extracted so the correct Patient can be identified (11020). Once the patient is identified, the data is standardized (11030) so that the data can be effectively used in the Data Command Center and then can be stored (11040). The last step in the process is to evaluate the data (11050) using a variety of mechanisms using advanced analytics as will be described in greater detail below.

In accordance with the present principles, there exist multiple criteria which affect the display and augmentation of data fields, considered by the inventors as Dynamic Data Fields, for a Data Command Center of the present principles. In some embodiments, the Data Command Center enables the medical records dashboard to intelligently alert by any means, gray out, expand, collapse, display, and/or hide columns, rows, fields, and/or any other portion of the medical records dashboard to show precisely w % bat a user wishes to display, or can alert by any means, gray out, expand, collapse, display, and/or hide columns, rows, fields, and/or any other portion of the medical records dashboard based on rules or triggers overriding the user's pre-determined display to show important details which the user should be made aware of. For example, in one embodiment, a Flowsheet including patient treatment and health information can be accessed from an EHR system using, in some embodiments, an icon/button, keystroke, or series of keystrokes, gesture, voice command, or other means, associated with at least one of the Data Command Center and the medical records dashboard. Upon accessing the Flowsheet, a set of Rules and Configurations associated with, for example, a Rules Engine, for example the Rules Engine 10180 of FIG. 3, can be evaluated to determine which data from the Flowsheet is to be displayed in a medical records dashboard of the present principles. For example, in some embodiments, the Rules Engine 10180 of FIG. 3 can include information on what data to display, and in turn what portions of the medical records dashboard to display, based on, including but not limited to, at least one of an identity of a medical care provider, an identity of a patient, a patient's medical history, a medical care provider's specialty, a user's custom configurations, patient appointment type, conditions of a patient, patient procedures, risk factors, diagnostic tests and/or results, future orders, future appointments, co-management status, predefined triggers, triggers generated in real time, values recorded, values not recorded, calculated values, and absolute values for display.

For example, in some embodiments in accordance with the present principles, Rules and Configurations can be predetermined and stored, for example, in the Rules Engine 10180 of FIG. 3, for determining which data of a Flowsheet and, as such, which portions of the medical records dashboard to alert by any means, gray out, expand, collapse, display, and/or hide based on known rules. Alternatively, or in addition, in some embodiments, a user can self-configure the medical records dashboard to display only certain portions or to hide certain data of a Flowsheet and, as such, which portions of the medical records dashboard to display or to hide using, for example, a user interface associated with the medical records dashboard. Alternatively, or in addition, data of the Flowsheet can contain an indicator (e.g., a flag) that can be identified by, for example, the Rules Engine 10180 of FIG. 3, for determining when and if a piece of data should be displayed, or can alert by any means, gray out, expand, collapse, display, and/or hide.

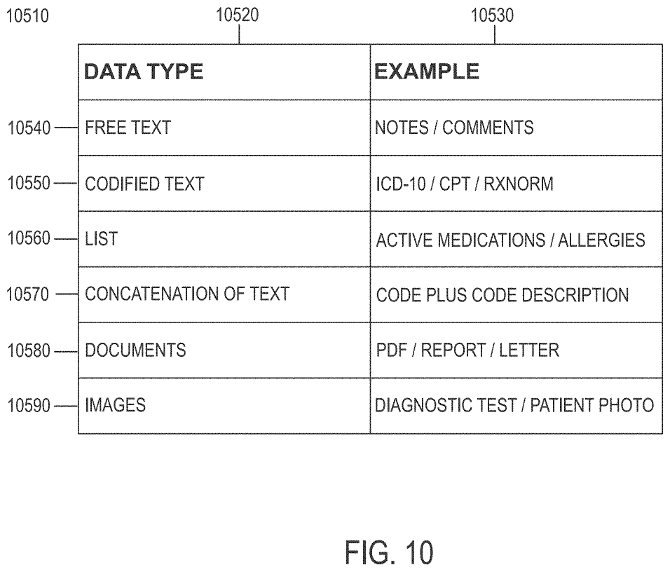

FIG. 10 depicts a Table listing of examples of different data types able to be accessed by a Data Command Center of the present principles such as the Data Command Center of FIG. 3. Data takes many forms, and is an overarching term for information, in general, regardless of format. In the case of Healthcare IT, certain standards exist, and data types which are common to the development of digital solutions. For the purpose of this document, several examples 10510 of data being accessed are listed below.

These data are listed by type 10520 and Example 10530. For example, such data can include but is not limited to:

Free Text 10540: Generally, any text that is entered free-hand without pertaining to a selectable list of options or industry standard. Notes and Comments fall into this category.

Codified Text 10550: Certain text within Healthcare IT applications are directly connected to industry standard code lists, such as ICD-10, CPT Codes, and RxNorm codes.

List 10560: Data elements can be comprised of lists of Problems, Medications, Allergies, or the like, and are stored and transported as such.

Concatenation of Text 10570: Not all data is maintained in normalized and standardized formats. As such, the ability to read and parse sections of text is important. In the case of data strings which can contain several elements, such as a code and a description, or a date and a doctor or location, certain elements can be important, while others can be ignored.

Documents 10580: Some data is simply stored in a precompiled format, such as a PDF or Word document. As such, the document can be stored and transported or can be parsed and data elements pulled out to populate discrete data fields.

Images 10590: Images can contain nothing more than the image, itself, and as such can be stored and transported in native format or converted to a different format. Images can also contain metadata and/or data elements within the image. As such, they can be parted, and data elements pulled out to populate discrete data fields.

In co-management, where different practices share information about the same patient, it is critical to identify, that the patient that is being shared is in fact the same person. There can be dozens of John Smiths and systems cross-reference by looking at the last name, the age, the gender, the zip code and perhaps the home address. But still, there can be confusion between patients. In medicine a user can take no chances that the user confuse one patient with the other and when patients travel from different offices or different EMRs and computer systems, the possibility of confusion is present.

In some embodiments, the Data Command Center of the present principles, such as the Data Command center 001 of FIG. 1, enables unique patient identification by incorporating patient medical history information. Current methods for identifying patients include matching Social Security Numbers (SSN) and Driver's License Numbers, where available. However, as privacy became more of a concern in the modern digital age, such data is becoming less available to medical care providers and their Practices. In addition, other methods for identifying patients can include identifying patients via First Name, Middle Name or Initial, Last Name, Age, Sex, Address, City, State, and Zip Code. Such information, however, is subject to flaws of human error, such as typos, human choice, such as a patient offering a nickname instead of the accurate name on a birth certificate or other identification. In addition, even having accurate patient information, it can still be difficult to distinguish between two people having the same name. Using such current methods, multiple systems are only able to match patients whose information is listed exactly the same in the multiple systems, a limitation which requires human intervention and prevents full automation of the process.

A subset of data exists within the Medical Community, as mandated by Meaningful Use 2014 and 2015 EHR Certification requirements specified in 45 CFR § 170.102, known as the Common Clinical Data Set (CCDS). The CCDS consists of patient information including, Patient Name. Sex, Date of birth, Race, Ethnicity, Preferred language, Smoking status, Medical Problems, Medications being taken, Medication allergies, Laboratory test(s) having been performed on the patient, values of the Laboratory result(s), Vital signs, Procedures, Care team member(s), Immunizations, Unique device identifier(s) for a patient's implantable device(s), Assessment and plan of treatment, Treatment Goals, Health concerns and the like.

CCDS was developed to encourage interoperability through the exchange of a common data set and is routinely shared between practices by means of the Direct Messaging Exchange, a secure messaging system by which Continuity of Care Document (CCD) or other document conforming to the Clinical Document Architecture (CDA) as defined in the 2014 and 2015 Certified EHR requirements. This is the current standard for Clinical Data transport between EHRs, thus between practices. The future requirement, Fast Healthcare Interoperability Resources (FHIR), expands on the clinical data set to include more discrete data points.

In accordance the present principles, the inventors propose to incorporate such additional data, such as the data supplied through the CCDS, to accurately identify unique patients using a combination of techniques including but not limited to a Common PII Matching technique, a Problems, Allergies, and Medications technique, a Doctors, Locations, and Procedures technique, and CCDS data technique.

In a Common PII Matching technique, none of the PII data may be valid given name changes, nicknames, and misspellings, as well as marriage and legal name changes, addresses and phone numbers change over time, and the increasing reluctance of patient and practice alike to maintain or share key identification numbers. At best, every data point would need to match exactly to ensure the closest match but can still fall short in the cases of same names such as in the case of George Forman's eight sons all named George Edward Foreman, if date of birth and suffix data was not present. Twins could make identification even more difficult. As evident, the Common PII Matching technique may not be reliable on its own for identifying unique patients.

In a Problems, Allergies, and Medications technique, a commonly shared data set which includes key conditions (Problems), allergies to certain medicines (Allergies), and specific medications (Medications), is compared to determine a profile of a patient which offers an additional level of accuracy by taking a loose match from PII and determining if that patient also has the same list of Medical Problems, Allergies, and Medications in a system for comparison. The likelihood that two people within similar PII, or lacking key aspects of PII, would also share the same Problems, Allergies, and Medications is a significant reduction in ambiguity. For instance, George Foreman's 3rd son may share certain genetic predispositions to Medical Problems and even share Allergies with a 1¹ son, but the likelihood that George Foreman's two sons would have been prescribed the same exact Medications for these and any other Problems they have is minimal.

In a Doctors, Locations, and Procedures technique, information from a document complying with the CCDA can be used for identifying a unique patient. For example, each CCD, or document complying with the CCDA, is required to have specific information in the Header of the document denoting the Care Provider, Date, and Location. The body of the document contains Procedures and relative Dates. The high accuracy enabled when comparing patients' Doctors, Locations, and Procedures is a product of the inability for a Doctor to see more than one patient at the exact same time, the unlikelihood of that even if the doctor saw more than one patient at the same time, and at the same location, the Doctor still would have little ability to perform the same procedure at the same time on more than one patient.

In a CCDS data technique, additional Data from the CCDS, when available, offers increased accuracy in patient identification and matching. That is, comparing patient information including at least Patient Name, Sex, Date of birth, Race, Ethnicity, Preferred language, Smoking status, Medical Problems, Medications being taken, Medication allergies, Laboratory test(s) having been performed on the patient, values of the Laboratory result(s), Vital signs, Procedures, Care team member(s), Immunizations, Unique device identifier(s) for a patient's implantable device(s), Assessment and plan of treatment, Treatment Goals, Health concerns and the like, among different patients, greatly increases the accuracy of unique patient identification.

In some embodiments of a Unique Patient Identification method of a Data Command Center in accordance with the present principles, a Unique Patient Identification algorithm collects every available Identification Point, validates the points for presence of data, and assigns each Identification point a level of accuracy as it pertains to Patient Matching. Presence of data points with High Accuracy are prioritized and validated. Each Exact match is scored for accuracy. Each Likely Match is appropriately scored for accuracy. Each data point with no matching counterpart is negatively scored. Presence of data points with Moderate Accuracy are then prioritized and validated. Each Exact match is scored for accuracy. Each Likely Match is appropriately scored for accuracy. Each data point with no matching counterpart is negatively scored. Moderate accuracy data points are scored lower than High accuracy data points. Presence of data points with Low Accuracy are then prioritized and validated. Each Exact match is scored for accuracy. Each Likely Match is appropriately scored for accuracy. Each data point with no matching counterpart is negatively scored. Low accuracy data points are score lower than Moderate accuracy data points.

Upon gathering and analyzing all available data for Unique Patient Identification, scores are tallied and compared to an acceptable Matching Threshold. In some embodiments of the present principles, the Matching Threshold is configured to clearly exceed a matching accuracy of current patient identification techniques with the inclusion of far more points of identification to compare. In some embodiments, the matching of the present principles can occur without the requirement of matching on current PII data. For example, George Edward Foreman IV may have been staying with a friend in Florida when he visited a doctor. Not wanting to be identified as the son of the famous boxer, he purposely listed his name as G. Foreman and address as the place he was staying. Date of birth may have been left blank. A positive identification can still be made, in accordance with the present principles, if the clinical data supplied matches with a high enough degree of accuracy clinical data stored for George Edward Foreman IV, such as the unique identifier on his knee replacement or the fact that a large number of Doctors, Locations, Procedures, Problems, Allergies. Medications, and Lab Results are found to be matching, while the name, address, and date of birth have non-matching counterparts.

A Unique Patient Identification algorithm of the present principles can reach a logical end when a positive match is determined, or no positive match can be made. In some embodiments, should no positive match be made, the patient and possible matches can be flagged for human intervention.

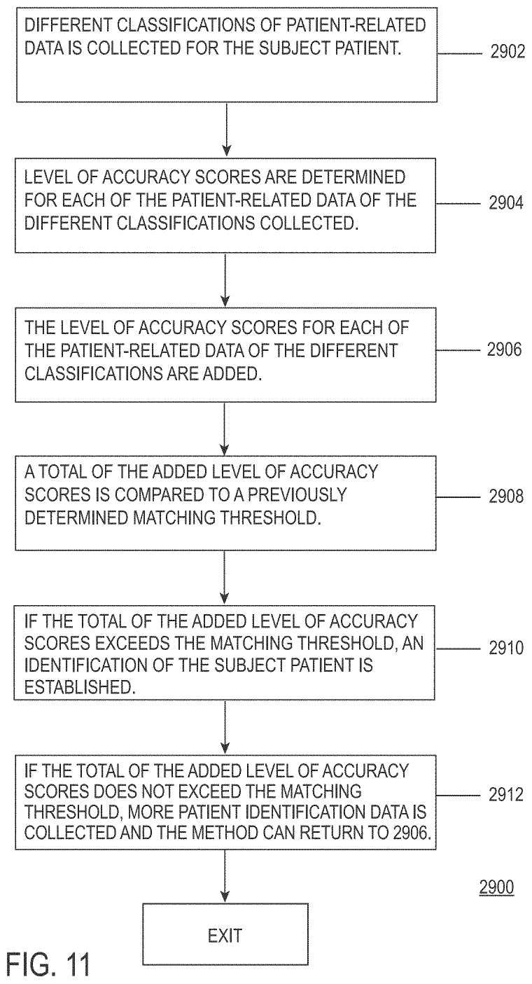

FIG. 11 depicts a flow diagram of a method for Unique Patient Identification for a subject patient in a Data Command Center including patient-related data received or derived from at least one patient database in accordance with an embodiment of the present principles. The method 2900 of FIG. 11 illustratively begins at 2902 during which different classifications of patient-related data is collected for the subject patient. For example, and as described above, in some embodiments, data from the Common Clinical Data Set and other sources can be collected to be used in patient identification techniques of the present principles. The method 2900 can proceed to 2904.

At 2904, level of accuracy scores are given for each of the patient-related data of the different classifications collected. The method 2900 can proceed to 2906.

At 2906, the level of accuracy scores for each of the patient-related data of the different classifications are added. The method 2900 can proceed to 2908.

At 2908, a total of the added level of accuracy scores is compared to a previously determined matching threshold. The method 2900 can proceed to 2910.

At 2910, if the total of the added level of accuracy scores exceeds the matching threshold, an identification of the subject patient is established. The method 2900 can proceed to 2912.

At 2912, if the total of the added level of accuracy scores does not exceed the matching threshold, more patient identification data is collected and the method 2900 can return to 2906. The method 2900 can then be exited.

The Command Center clinical decision support logic is implemented in a variety of methods. Pre-authorization, referral management, claims scrubbing, medical necessity checking for compliance with governmental and insurance regulations, and similar rules are embodied in the system through the use of third party systems. Internally, the Rules Engine (10180 of FIG. 3) is an implementation of the if-then style of clinical decision support.

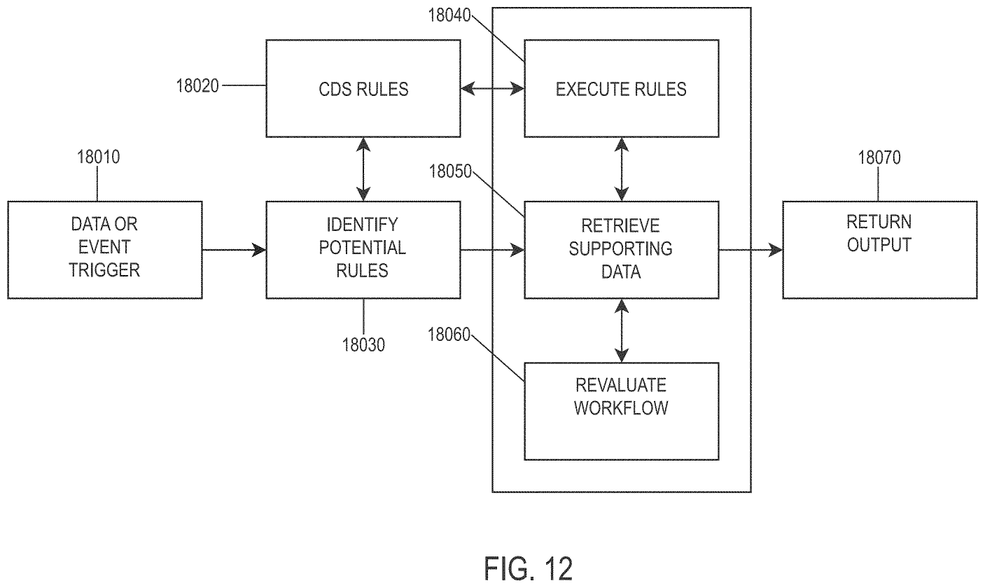

More complex clinical decision support is illustrated in FIG. 12. In FIG. 12, the support is handled through the use of a RETE style (pattern matching, rules-based) algorithm. A RETE rules engine or inference engine embodies a set of rules built around a data model whereby when an event is triggered 18010 (data input or a new day starts) potential rules 18020 are identified (18030) that relate to the data/event. If rules are identified, they are executed (18040) along with the evaluation of workflow rules 18050 using supporting data retrieved (18060) as needed from the patient, insurance, clinical, financial, and imaging data repositories. Once complete, the outcome is returned (18070) to the requesting system for processing. Typically, the output is displayed on the screen for the user to consume or for storage in the designated database in the Command Center. In exemplary embodiments, the Command Center CDS illustrated in FIG. 12 is based on the Health Level (HL7) and Object Management Group (OMG) Decision Support Standards making it flexible and compatible with other similar systems. Those skilled in the art will appreciate that the inference engine may implement conventional artificial intelligence techniques such as those provided commercially by Watson Health and Truven Health Analytics, Inc. to process received data in connection with data repositories to provide diagnostic feedback and the like.

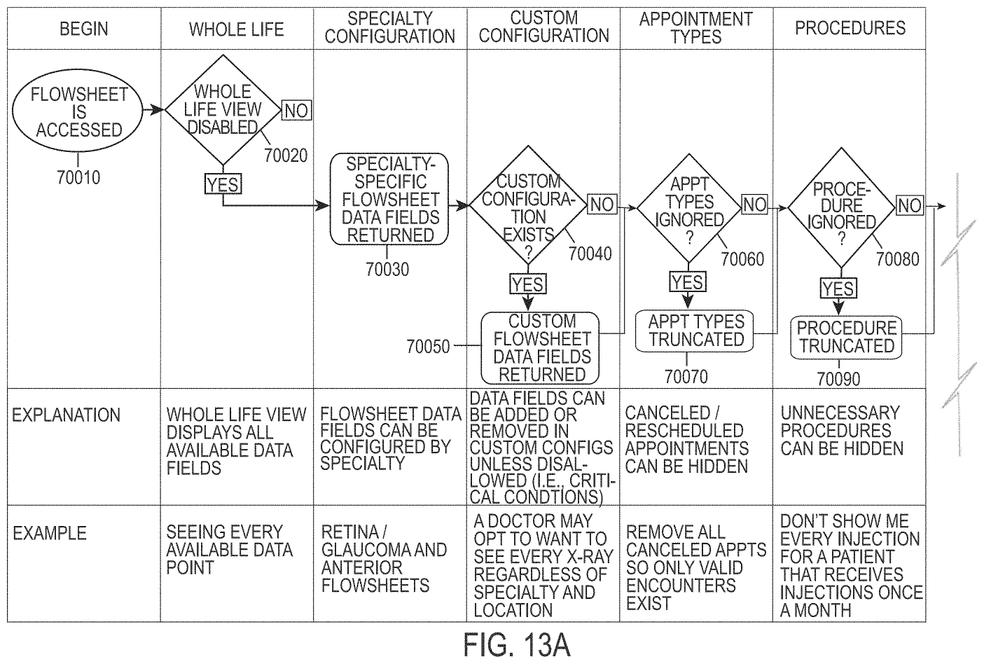

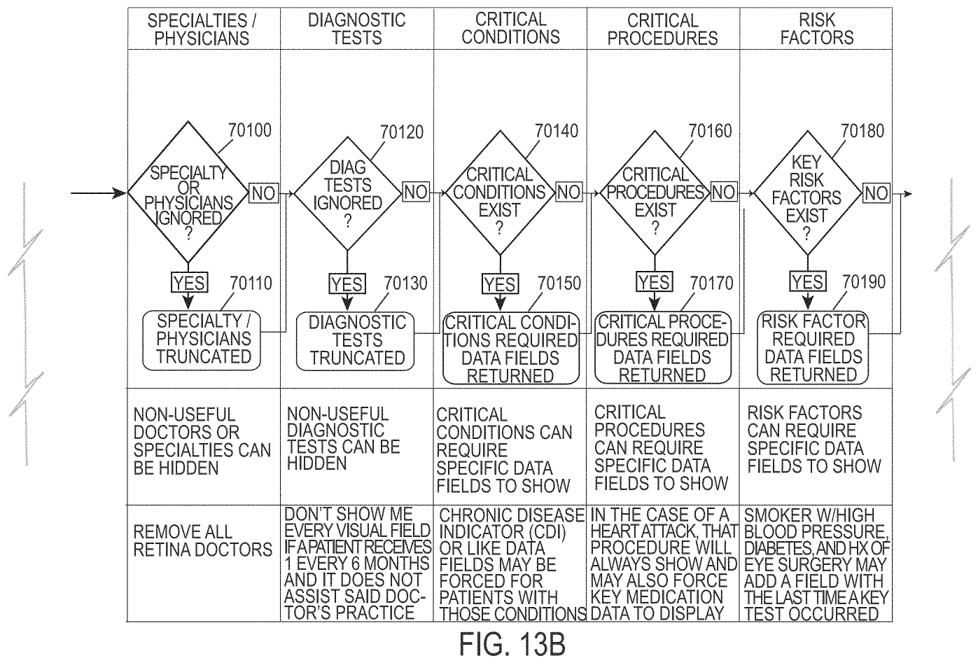

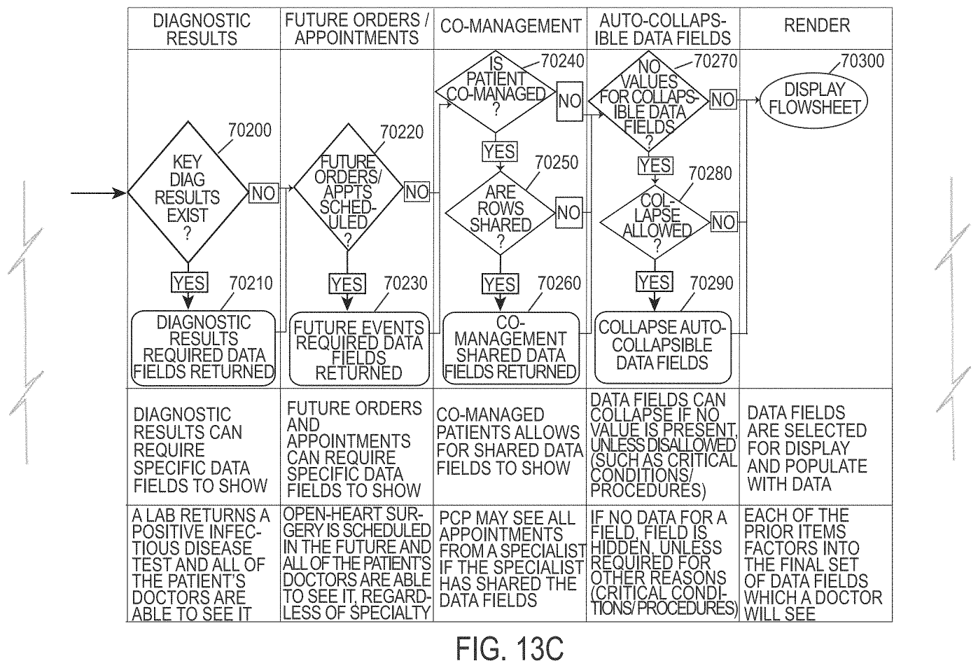

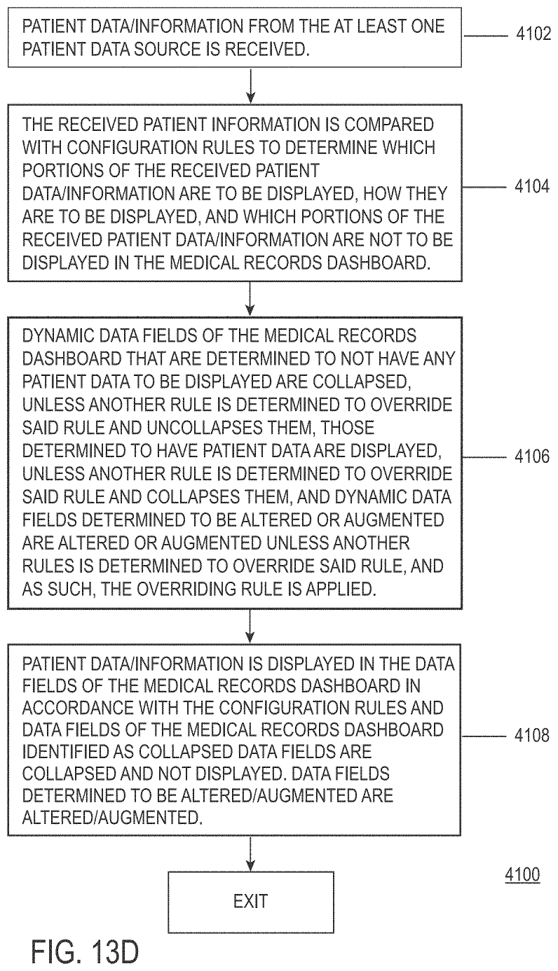

FIGS. 13A, 13B, and 13C, collectively referred to as FIG. 13 herein, depict a workflow diagram of an alternate embodiment of a process for intelligently alerting by any means, graying out, expanding, collapsing, displaying, and/or hiding columns, rows, fields, and/or any other portion of the medical records dashboard based on rules. In the embodiment depicted in FIG. 13, the process begins at 70010 during which a Flowsheet including patient treatment and health information is accessed from, for example, an EHR system. The process illustratively proceeds to 70020. At 70020, it is determined if, what the inventors refer to as a “Whole Life View”, is disabled. More specifically, at 70020 it is determined if all the data in the Flowsheet should be displayed in the medical records dashboard. If Whole Life View is disabled, the process proceeds to 70240 during which all of the data from the Flowsheet is displayed in the medical records dashboard. If not, the process illustratively proceeds to 70030.

At 70030, it is determined if at least one Specialty Configuration exists. For example, in some embodiments a Specialty Configuration can include a configuration based on the specialty of a medical care provider. If so, the process proceeds through 70030 during which all Specialty Configurations are identified such that the data from the Flowsheet can be filtered to only display data associated with identified Specialty Configurations. For example, as previously described, in some embodiments, information associated with medical care provider specialties and data to be displayed and hidden in the medical records dashboard dependent on the specialties can be predetermined and stored in the Rules Engine 10180 of FIG. 3. In accordance with the present principles, Specialty Configurations can require certain portions, columns, and/or rows of the medical records dashboard to be displayed or hidden. After the Specialty Configurations are identified and/or if it is determined that a Specialty Configuration does not exist, the process illustratively proceeds to 70040. In accordance with the present principles, data from the Flowsheet to be displayed in or hidden from the medical records dashboard can be filtered using the identified Specialty Configurations.

At 70040, it is determined if at least one Custom Configuration exists. If so, the process proceeds to 70050 during which all Custom Configurations are identified such that the data from the Flowsheet is filtered to only display or hide, collapse or expand, gray out or alert by any means, data associated with the identified Custom Configurations. For example, in some embodiments custom configurations and data to be displayed in, hidden from, or alerted by any means, the medical records dashboard dependent on the custom configurations can be predetermined and stored in the Rules Engine 10180 of FIG. 3. Alternatively, or in addition, in some embodiments, a user can use a user interface associated with the medical records dashboard to create and/or identify custom configurations. In accordance with the present principles, Custom Configurations can require certain portions, columns, and/or rows of the medical records dashboard to be displayed or hidden. After the Custom Configurations are identified and/or if it is determined that a Custom Configuration does not exist, the process illustratively proceeds to 70060. In accordance with the present principles, data from the Flowsheet to be displayed in or hidden, collapsed or expanded, grayed out or alerted by any means, from the medical records dashboard can be filtered using the identified Custom Configurations.

At 70060, it is determined if at least one predefined Appointment Type exists. That is, in some embodiments, appointment types can be identified that, no matter what rules indicate that certain data should not be displayed or hidden, collapsed or expanded, grayed out or alerted by any means, the identified appointment types are to be displayed or hidden, collapsed or expanded, grayed out or alerted by any means, in at least one location of a medical records dashboard of the present principles. In some embodiments, appointment types can be identified and stored in, for example, a storage accessible by the Rules Engine 10180 of FIG. 3. Alternatively, or in addition, a user can identify appointment types using a user interface associated with a medical records dashboard of the present principles. If it is determined that at least one specified appointment type exists, the process proceeds to 70070 during which the appointment types are identified such that any data from the Flowsheet identified as a specified appointment type can be displayed or hidden, collapsed or expanded, grayed out or alerted by any means, in at least one portion of a medical records dashboard of the present principles. In accordance with the present principles, appointment types can require certain portions, columns, and/or rows of the medical records dashboard to be displayed or hidden, collapsed or expanded, grayed out or alerted by any means. After the appointment types are identified or if it is determined that a specified appointment type does not exist, the process illustratively proceeds to 70080.

At 70080, it is determined if at least one predefined Procedure exists. That is, in some embodiments, procedures can be identified that, no matter what rules indicate that certain data should not be displayed or hidden, collapsed or expanded, grayed out or alerted by any means, the identified procedures are to be displayed or hidden, collapsed or expanded, grayed out or alerted by any means, in at least one location of a medical records dashboard of the present principles. In some embodiments, procedures can be identified and stored in, for example, a storage accessible by the Rules Engine 10180 of FIG. 3. Alternatively, or in addition, a user can identify procedures using a user interface associated with a Data Command Center of the present principles. If it is determined that at least one specified procedure exists, the process proceeds to 70090 during which the procedures are identified such that any data from the Flowsheet identified as a specified procedure can be displayed or hidden, collapsed or expanded, grayed out or alerted by any means, in at least one portion of a medical records dashboard of the present principles. In accordance with the present principles, procedures can require certain portions, columns, and/or rows of the medical records dashboard to be displayed or hidden, collapsed or expanded, grayed out or alerted by any means. After the procedures are identified or if it is determined that a specified procedure does not exist, the process illustratively proceeds to 70100.

At 70100, it is determined if at least one predefined Specialty or Physician exists. That is, in some embodiments, specialties or physicians can be identified that, no matter what rules indicate that certain data should not be displayed or hidden, collapsed or expanded, grayed out or alerted by any means, the identified specialties or physicians are to be displayed or hidden, collapsed or expanded, grayed out or alerted by any means, in at least one location of the medical records dashboard. In some embodiments, specialties or physicians can be identified and stored in, for example, a storage accessible by the Rules Engine 10180 of FIG. 3. Alternatively, or in addition, a user can identify specialties or physicians using a user interface associated with a medical records dashboard of the present principles. If it is determined that at least one specified specialty or physician exists, the process proceeds to 70110 during which the specialties or physicians are identified such that any data from the Flowsheet identified as a specified specialty or physician can be displayed or hidden, collapsed or expanded, grayed out or alerted by any means. In accordance with the present principles, specialties or physicians can require certain portions, columns, and/or rows of the medical records dashboard to be displayed or hidden, collapsed or expanded, grayed out or alerted by any means. After the specialties and physicians are identified or if it is determined that a specified specialty or physician does not exist, the process illustratively proceeds to 70120.

At 70120, it is determined if at least one predefined Diagnostic Test exists. That is, in some embodiments, diagnostic tests can be identified that, no matter what rules indicate that certain data should not be displayed or hidden, collapsed or expanded, grayed out or alerted by any means, the identified diagnostic tests are to be displayed or hidden, collapsed or expanded, grayed out or alerted by any means, in at least one location of a medical records dashboard of the present principles. In some embodiments, diagnostic tests can be identified and stored in, for example, a storage accessible by the Rules Engine 10180 of FIG. 3. Alternatively, or in addition, a user can identify diagnostic tests using a user interface associated with a medical records dashboard of the present principles. If it is determined that at least one specified diagnostic test exists, the process proceeds to 70130 during which the diagnostic tests are identified such that any data from the Flowsheet identified as a specified diagnostic test can be displayed or hidden, collapsed or expanded, grayed out or alerted by any means, in at least one portion of a medical records dashboard of the present principles. In accordance with the present principles, diagnostic tests can require certain portions, columns, and/or rows of the medical records dashboard to be displayed or hidden, collapsed or expanded, grayed out or alerted by any means. After the diagnostic tests are identified or if it is determined that a specified diagnostic test does not exist, the process illustratively proceeds to 70140.

At 70140, it is determined if at least one Critical Condition exists. That is, in some embodiments, critical conditions can be identified that, no matter what rules indicate that certain data should not be displayed or hidden, the identified critical conditions are to be displayed in at least one location of a medical records dashboard of the present principles. In some embodiments, Critical Conditions can be identified and stored in the Rules Engine 10180 of FIG. 3. Alternatively, or in addition, a user can identify Critical Conditions using a user interface associated with the medical records dashboard 400. If it is determined that at least one Critical Condition exists, the process proceeds to 70150 during which the Critical Conditions are identified such that any data from the Flowsheet identified as a Critical Condition can be displayed. In accordance with the present principles, Critical Conditions can require certain portions, columns, and/or rows of the medical records dashboard to be displayed or hidden. After the Critical Conditions are identified or if it is determined that a Critical Condition does not exist, the process illustratively proceeds to 70160.

At 70160, it is determined if at least one Critical Procedure exists. That is, in some embodiments, critical procedures can be identified that, no matter what rules indicate that certain data should not be displayed or hidden, data associated with the identified critical procedures are to be displayed in at least one location of the medical records dashboard 400. In some embodiments, Critical Procedures can be identified and stored in the Rules Engine 10180 of FIG. 3. Alternatively, or in addition, a user can identify Critical Procedures using a user interface associated with the medical records dashboard 400. If it is determined that at least one Critical Procedure exists, the process proceeds to 70170 during which data associated the Critical Procedures are identified such that any data from the Flowsheet identified as being associated with a Critical Procedure can be displayed in at least one portion of the medical records dashboard 400. In accordance with the present principles, Critical Procedures can require certain portions, columns, and/or rows of the medical records dashboard to be displayed or hidden. After the Critical Procedures are identified or if it is determined that a Critical Procedure does not exist, the process illustratively proceeds to 70180.

At 70180, it is determined if at least one Risk Factor exists. That is, in some embodiments, Risk Factors can be identified that, no matter what rules indicate that certain data should not be displayed or hidden, the identified Risk Factors are to be displayed in at least one location of a medical records dashboard of the present principles. In accordance with the present principles, Risk Factors can require certain portions, columns, and/or rows of the medical records dashboard to be displayed or hidden. For example, a smoker with high blood pressure, and diabetes having an identified Risk Factor for a heart attack can require a visual field column with an alert to be displayed in at least a portion of the medical records dashboard 400. In some embodiments, Risk Factors can be identified and stored in, for example, a storage accessible by the Rules Engine 10180 of FIG. 3. Alternatively, or in addition, a user can identify Risk Factors using a user interface associated with the medical records dashboard. If it is determined that at least one Risk Factor exists, the process proceeds to 70190 during which the Risk Factors are identified such that any data from the Flowsheet identified as identifying a Risk Factor can be displayed in at least one portion of the medical records dashboard. After the Risk Factors are identified or if it is determined that a Risk Factor does not exist, the process illustratively proceeds to 70200.

At 70200, it is determined if at least one Key Diagnostic Result exists. That is, in some embodiments, Diagnostic Results that are considered Key can be identified that, no matter what rules indicate that certain data should not be displayed or should be hidden, data associated with the identified Key Diagnostic Results are to be displayed in at least one location of a medical records dashboard of the present principles. In accordance with the present principles, Key Diagnostic Results can require certain portions, columns, and/or rows of the medical records dashboard to be displayed or hidden. For example, if a lab returns a positive infectious disease test, data associated with that Key Diagnostic Result can be caused to be displayed in at least a portion of the medical records dashboard. In some embodiments, Key Diagnostic Results can be identified and stored in, for example, a storage accessible by the Rules Engine 10180 of FIG. 3. Alternatively, or in addition, a user can identify Key Diagnostic Results using a user interface associated with the medical records dashboard. If it is determined that at least one Key Diagnostic Results exists, the process proceeds to 70210 during which the Key Diagnostic Results are identified such that any data from the Flowsheet identified as being associated with a Key Diagnostic Results can be displayed in at least one portion of the medical records dashboard 400. After the Key Diagnostic Results are identified or if it is determined that a Key Diagnostic Results does not exist, the process illustratively proceeds to 70220.

At 70220 of the embodiment of FIG. 13, it is determined if at least one Future Order/Appointment exists. That is, in some embodiments, Future Orders/Appointments can be identified that, no matter what rules indicate that certain data should not be displayed or should be hidden, data associated with the identified Future Order/Appointment are to be displayed in at least one location of a medical records dashboard of the present principles. In accordance with the present principles, Future Orders/Appointments can require certain portions, columns, and/or rows of the medical records dashboard to be displayed or hidden. For example, if an Open-heart surgery is scheduled for the future, it can be desirable for all medical care providers to see the scheduled Open-heart surgery in at least a portion of the medical records dashboard regardless of a medical care provider's specialty. In some embodiments, Future Orders/Appointments can be identified and stored in, for example, a storage accessible by the Rules Engine 10180 of FIG. 3. Alternatively, or in addition, a user can identify Future Orders/Appointments using a user interface associated with the medical records dashboard. If it is determined that at least one Future Order/Appointment exists, the process proceeds to 70230 during which the Future Orders/Appointments are identified such that any data from the Flowsheet identified as being associated with a Future Order/Appointment can be displayed in at least one portion of the medical records dashboard. After the Future Orders/Appointments are identified or if it is determined that a Future Order/Appointment does not exist, the process illustratively proceeds to 70240.

At 70240, it is determined if Co-Management of at least one patient is allowed and if patient information sharing is allowed. That is, in some embodiments, Co-Management of patients can require certain portions, columns, and/or rows of the medical records dashboard to be shared or hidden amongst different users/medical care providers. For example, at 70250, if a medical records dashboard in accordance with the present principles is being used by multiple medical care providers to care for a patient, the patient's primary care physician is able to see lab results from a specialist if the specialist has shared at least the relevant portions of a medical records dashboard. In some embodiments, patient data/information to be shared and, as such, portions of a medical records dashboard to be shared can be identified and stored in, for example, a storage accessible by the Rules Engine 10180 of FIG. 3. Alternatively, or in addition, a user can identify patient data/information to be shared and, as such, portions of a medical records dashboard to be shared using a user interface associated with the medical records dashboard. If it is determined that Co-Management of at least one patient exists and if patient information sharing is allowed, the process proceeds to 70260 during which the existence of Co-Management of at least one patient and patient information sharing is identified such that any data from the Flowsheet identified as being associated with Co-Management and patient information sharing can be displayed in at least one portion of the medical records dashboard 400. After the Co-Management and patient information sharing is identified or if it is determined that Co-Management and patient information sharing does not exist, the process illustratively proceeds to 70270.

In the embodiment of FIG. 13, at 70270, it is determined if any of the collapsible portions, columns, and/or rows of the medical records dashboard contain no respective values (i.e., are empty). If it is determined that collapsible portions, columns, and/or rows of the medical records dashboard contain no respective values, the process proceeds to 70280 during which it is determined if there are any overriding rules to disallow collapsing or hiding portions, columns, and/or rows of the medical records dashboard. If it is determined that collapsible portions, columns, and/or rows of the medical records dashboard contain no respective values, and there are no overriding rules, the collapsible portions, columns, and/or rows of a medical records dashboard of the present principles containing no respective values proceed to 70290 and can be collapsed or hidden from display on a least a portion of the medical records dashboard. After all of the display configurations have been determined as described above, at 70300 the data of the Flowsheet to be displayed, as determined by the process of FIG. 13 described above, is displayed in the medical records dashboard. The process can then be exited.

In accordance with the present principles and as described above, in some embodiments, rules determine portions, columns, and/or rows of the medical records dashboard to expand or display based on predefined criteria, and also determine portions, columns, and/or rows of the medical records dashboard to collapse or hide based on the predefined criteria, and can also determine portions, columns, and/or rows of the medical records dashboard to flag or emphasize such as by highlighting, bolding or otherwise calling attention to records based on the predefined criteria. For example, in some embodiments, the entirety of a patient's accessible records can be viewed. In some embodiments, the entirety of a patient's accessible records is evaluated against specialty and user-specific configuration criteria (e.g., Rules), actively collapsing or hiding portions, columns, and/or rows of the medical records dashboard deemed unnecessary for a user or specialty and actively enabling the display of portions, columns, and/or rows of the medical records dashboard deemed relevant to the user or specialty. In some embodiments, an intelligent Rules system actively determines which portions, columns, and/or rows of the medical records dashboard to display based on a user, a user's specialty, a patient, a patient conditions, a patient procedures, risk factors, diagnostic results, future orders, future appointments, values recorded, values not recorded, calculated values, and absolute values for display. In another embodiment, shared portions, columns, and/or rows of the medical records dashboard between medical care providers and facilities can be added or expanded based on preconfigured or point-of-sharing decisions made by the sharing medical care providers.

Although the embodiment of the process for intelligently expanding, collapsing, displaying, hiding, graying out, and/or alerting columns, rows and/or any other portion of the medical records dashboard of the present principles described with reference to FIG. 13 illustratively comprises specific Rules-based configurations, other embodiments of the process in accordance with the present principles can comprise any combination of some or all of the described Rules-based configurations and can also comprise other Rules-based configurations. Even further, those skilled in the art will appreciate that the order of operations denoted in the process above with reference to FIG. 13 can be non-linear and optimized based on usage and workflow. That is, order, inclusion, and omission can be intelligently determined based on accessibility of data, predefined configurations, real-time user selection, custom configurations, preferred practice patterns, and/or workflow.

In addition, although in the embodiment of the process for intelligently expanding, collapsing, displaying, hiding, graying out, and/or alerting columns, rows and/or any other portion of the medical records dashboard of the present principles described with reference to FIG. 13, the Rules are described as being stored in a storage accessible to the Rules Engine 10180 of FIG. 3, those skilled in the art will appreciate that rules and configurations of a process of the present principles can be stored in tables, accessed remotely via API or other digital communications technology, or generated on-the-fly as the result of calculations during the operations. Rules and configurations can be stored within the application or reference outside data sources. Rules and configurations can be altered by the user, in some embodiments, by the application, in some embodiments, and/or by outside resources.

In addition, although in the embodiment of the process for intelligently expanding, collapsing, displaying, hiding, graying out, or alerting columns, rows and/or any other portion of the medical records dashboard of the present principles described with reference to FIG. 13, it is described that upon rendering the Flowsheet, data populates within the columns specified, in some embodiments, further rules and configurations can apply post-rendering, based on data returned and/or calculated within columns. In addition, in some embodiments, manual manipulation allows for human interaction with the finally determined dataset. As such, a user can acknowledge and remove portions, columns, and/or rows of the medical records dashboard once they have been rendered. Removal of such portions, columns, and/or rows of the medical records dashboard can be one-time, or permanent unless a subsequent event retriggers the rendering of those portions, columns, and/or rows of the medical records dashboard, and such rendering can be patient-specific, provider-specific, location-specific, or otherwise tied to an event, condition, or trigger.

In one example of the process of the present principles, a dentist can access a Flowsheet for a patient with a rare blood disorder. As a dentist, the returned set of data to be displayed in accordance with a process of the present principles would ordinarily include data germane to dentistry, collapsing or hiding certain portions, columns, and/or rows of the medical records dashboard with no values present and/or deemed unnecessary. The dentist can have also chosen not to view certain portions, columns, and/or rows of the medical records dashboard as a matter of practice. In accordance with embodiments of the present principles, as a patient with a rare blood disorder, additional portions, columns, and/or rows of the medical records dashboard could be added to the display to reflect the patient's condition of the rare blood disorder and such information could be emphasized such as by highlighting/flagging to alert a user as to the importance of the information being displayed.

In another example, an ophthalmologist sees a diabetic patient with no diagnostic testing for a chronic illness. As an ophthalmologist, the patient data ordinarily returned for display by a process of the present principles would ordinarily include data germane to ophthalmology, collapsing or hiding certain portions, columns, and/or rows of the medical records dashboard with no values present or data deemed unnecessary for display by the process. In some embodiments, the ophthalmologist can have also chosen not to view certain columns as a matter of practice. As a patient with a lapse in testing and underlying condition requiring testing, portions, columns, and/or rows of the medical records dashboard having no value present which would normally be collapsed/hidden, could now be expanded/displayed, and highlighted or flagged or otherwise emphasized to draw the attention of a user to the lack of testing having been performed on the patient.

In a third example, a primary care physician (PCP) may wish to view an entire patient history. The patient history can consist of patient care provided by the PCP, patient care provided by doctors in the same office as the PCP, and patient care provided by specialists outside the practice that co-manage the patient and have shared data with the PCP. In this arrangement, the entire dataset is provided for viewing on the medical records dashboard for care provided by the PCP and doctors within the same practice, and a shared dataset can be provided for viewing on the medical records dashboard for care provided by the specialists. Columns with no values can be collapsed or hidden if no value exists as described above.

FIG. 13D depicts a flow diagram of a method for rules-based data display in a data command center comprising a medical records dashboard including one or more windows including information received or derived from at least one patient database, the medical records dashboard comprising a display on a screen, using the one or more windows, of at least one of medical services, clinical data, examination findings, diagnostic tests, and the procedures performed on one or more patients, the one or more windows comprising a plurality of dynamic data fields for displaying the information received or derived from the at least one patient database, wherein the at least one of the medical services, the clinical data, the examination findings, the diagnostic tests, and the procedures are arranged in rows or columns on the screen according to at least one of a time and a date that the medical services, the clinical data, the examination findings, the diagnostic tests and the procedures were performed on the one or more patients, the method beginning at 6602 during which patient data/information from the at least one patient database is received. The method can proceed to 6604.

At 6604, the received patient information is compared with configuration rules to determine which portions of the received patient data/information are to be displayed and which portions of the received patient data/information is not to be displayed in the medical records dashboard. The method can proceed to 6606.

At 6606, dynamic data fields of the medical records dashboard that are determined to not have any patient data to display are identified as collapsed data fields, unless another rule is determined to override said rule and uncollapses/expands them. Those determined to have patient data are displayed, unless another rule is determined to override said rule and collapses them. Those determined to be altered, augmented, and/or emphasized are altered or augmented, unless another rule is determined to override said rule, and as such, the overriding rule is applied. The method can proceed to 6608.

At 6608, Patient data/information is displayed in the data fields of the medical records dashboard in accordance with the configuration rules and data fields of the medical records dashboard identified as collapsed data fields are collapsed and not displayed. Data fields determined to be altered/augmented are altered/augmented. The method can then be exited.

In some embodiments the dynamic data fields identified as intelligently alerted by any means, grayed out, expanded, collapsed, displayed, and/or hidden columns, rows, fields, and/or any other portion of the medical records dashboard comprise at least one of a column, row, or panel of a medical records dashboard of the present principles.

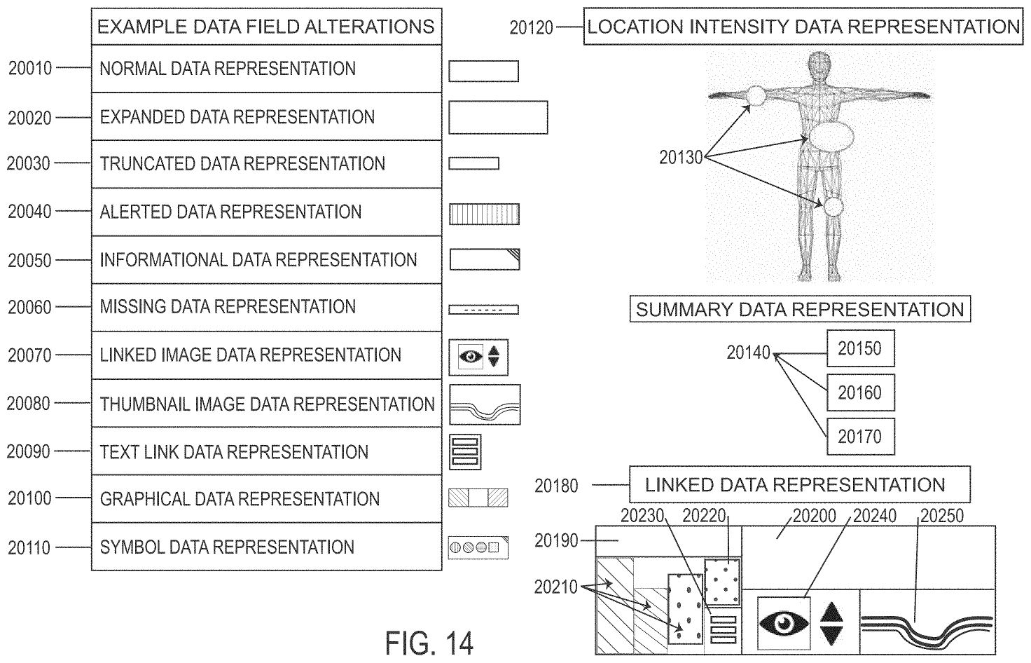

FIG. 14 depicts a graphical representation of examples of altered/augmented data representation in accordance with Dynamic Data Representation in accordance with an embodiment of the present principles. Dynamic Data Representation in accordance with the present principles can include but are not limited to:

Normal Data Representation 20010: As an example, a normal data field can simply exist as a box to contain data. It can also be represented as an icon, image, diagram, or any other means by which the data could be displayed without any alteration or augmentation.

Expanded Data Representation 20020: A data field can be expanded to show more data, or to draw attention to data contained within.

Truncated Data Representation 20030: A data field can be shrunken to hide less important data or to show the lack of data present.

Alerted Data Representation 20040: A data field can be highlighted by, for example, color, to draw attention to or otherwise emphasize incorrect data, data exceeding a threshold, or critically important data.

Informational Data Representation 20050: An highlighted or otherwise emphasized notification, such as the corner notification of FIG. 14, can be displayed and an associated note can identify more information as to why the notification is present.

Missing Data Representation 20060: A data field can have a series of dashes to indicate that although the data field was expected, the field is not filled to indicate missing data such as canceled or missed appointment data.

Linked Image Data Representation 20070: A data field can contain an icon and even a grading system to show that the field enables direct access to an underlying image, and can also indicate whether the image indicates whether there is an improvement or degradation from a prior image.

Thumbnail Image Data Representation 20080: A thumbnail of an image can be present within a data field offering a quick snapshot of, and direct access to, an underlying image.

Text Link Data Representation 20090: A data field can contain an icon to show direct access to text via the data field.

Graphical Data Representation 20100: A data field can represent underlying information as a graphical representation of the data.

Symbol or Icon Data Representation 20110: A data field can use a series of symbols or icons to represent underlying data in a way that the end user can interpret the symbols or icons to understand the underlying data.

Location Intensity Data Representation 20120: Data Representation is a general term to describe how to display an area which represents data. As such, representing the importance of key events in a graphical representation of a human body is also relevant. Differing representations can show location and intensity or importance of key areas being called out. In 20130, three different size and color data representations are depicted, which illustratively implement size and color to denote a relative intensity or importance of the data.

Summary Data Representation 20140-20170: Summary Data Representation illustrates a single data representation 20140 comprised of multiple data sources 20150-20160, with the ability to collate and summarize more than a single data source in a single representation. Summary data representations can offer total counts, highest and lowest values, best/worst values, and interaction within underlying data.

Linked Data Representation 20180: Linked Data Representation comprises multiple data representations in a combined representation. The example of Linked Data Representation 20180 as illustrated shows a Normal data representation 20190, next to an Expanded data representation 20200, above multiple Graphical data representations 20210, an Alerted data representation 20220, Linked Text data representation 20230, Linked Image data representation 20240, and a Thumbnail Image data representation 20250. Each individual data representation may affect the representation of any other, such that an alert 20220 can enlarge based on changes to the source data represented graphically 20210.

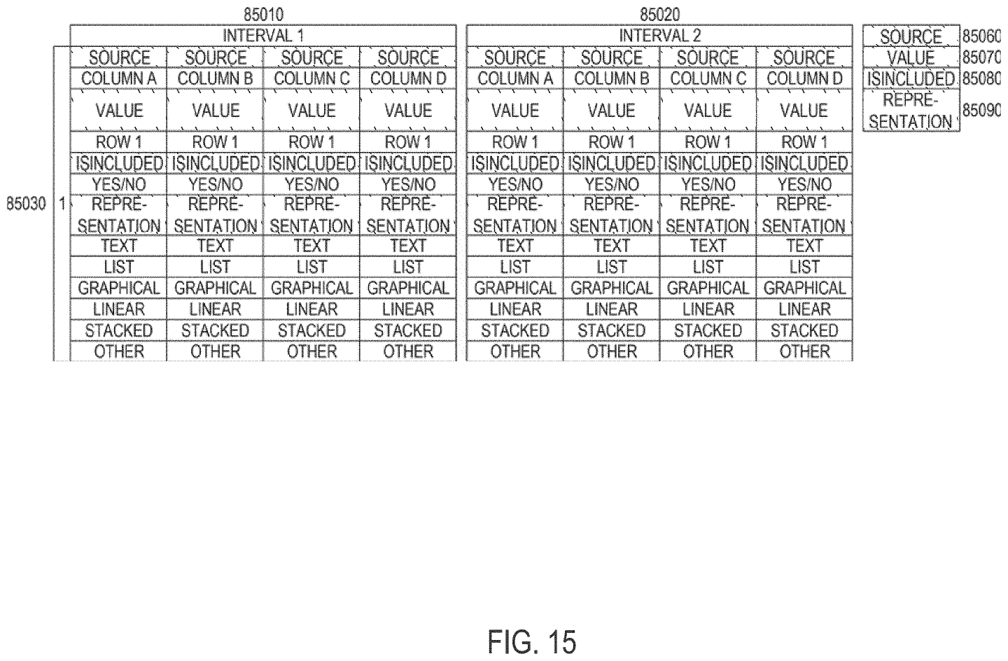

FIG. 15 depicts a Table depicting example configurations (data visualizations) of dynamic data fields in accordance with an embodiment of the present principles. In the embodiment of FIG. 15, data visualization is achieved with a series of configurations to determine what and how to display. For example, in one embodiment, Source Data (85060) consists of a Value (85070), an Inclusion/Exclusion Rule (85080), and a Visual Representation Configuration (85090). The data can consist of one type (85030), a series of data points collected, values captured, validated for inclusion, and visualized across 2 intervals (85010, 85020), correlated against additional types, a series of separate data points collected, values captured, validated for inclusion, and visualized across the same 2 intervals (85010, 85020).

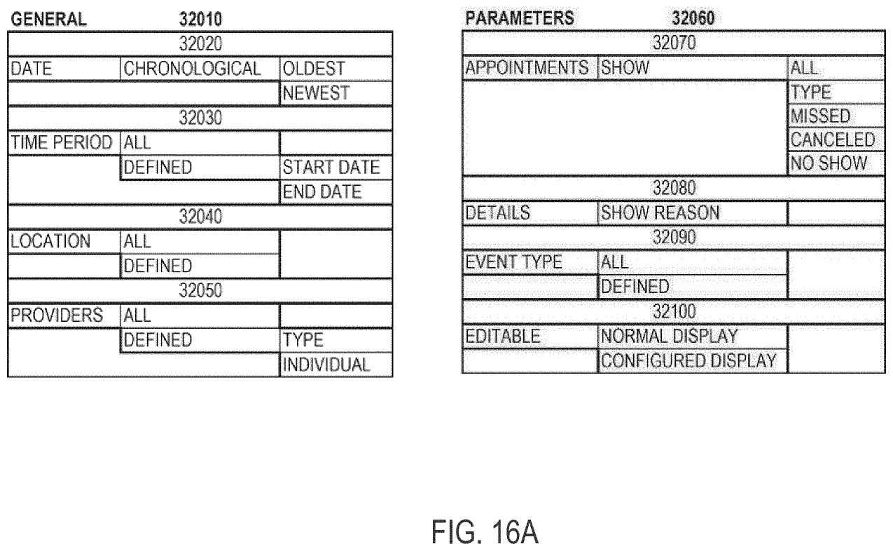

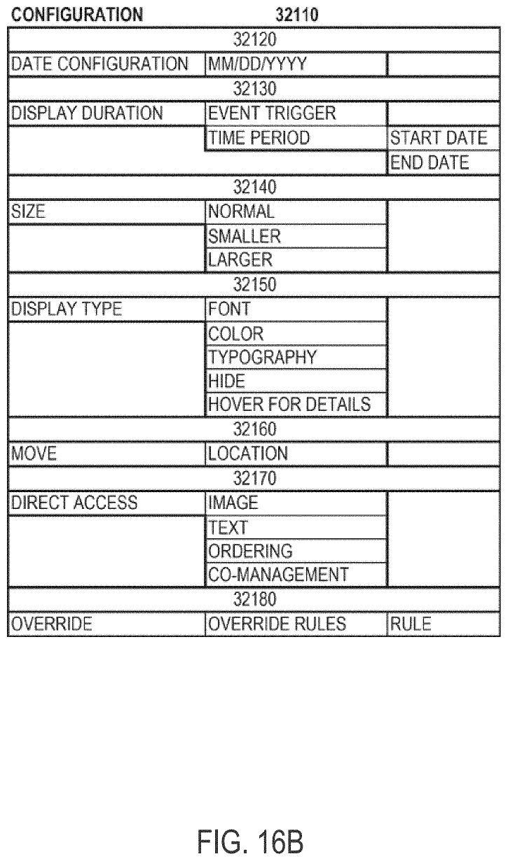

FIGS. 16A and 16B, (Hereinafter FIG. 16) depicts a graphical representation of Dynamic Data Field Example Configurations. In some embodiments, in order to accomplish dynamic data representation, a series of configuration rules must be employed. An example of such rules are illustrated with respect to the embodiment of FIG. 16. In this example, configuration has been shown with three panels, General Configuration 32010, denoting high-level choices as to what will be displayed, Parameters 32060, denoting when to augment display of data, and Configuration 32110, describing how to augment display of data. It should be noted that in some embodiments such configuration can include more or less sections or options within sections, based on use case and requirements.

Under General Configuration 32010. Date Configuration 32020 is displayed with options for Chronological ordering from Oldest to Newest or Newest to Oldest. Time Period 32030 enables the configuration to show all available data, or just data within a defined start and end date. Location 32040 enables refinement of which data will be displayed for example, for a specific medical provider practice/specialty. Providers 32050 enables specification of individual or types of providers.

Parameters 32060 address reasons to show, hide, or augment data. Appointments 32070 enables user defined types of appointments to be configured. Details 32080 determines if more information should be displayed about the underlying data. Event Type 32090 enables augmentation to be defined based on important, or any, event. Editable 32100 determines if such data should be accessible for alteration, insertion, deletion, or other modifications.

Configuration 32110 addresses how to augment underlying data. Date Configuration 32120 enables for date format to be specified, when date is present. Display Duration 32130 can be used to define a time period within which data will display or the tying of the duration of display to an event or action. Size 32140 enables the ability to make data appear larger or smaller than standard display. Display Type 32150 enables for augmentation of underlying data by enabling the data to have visual and/or typographic alterations. Move 32160 enables data to appear in a different location. Direct Access 32170 enables data to link to other data, images, or access panels. Override 32180 enables configurations to override other, predefined configurations.

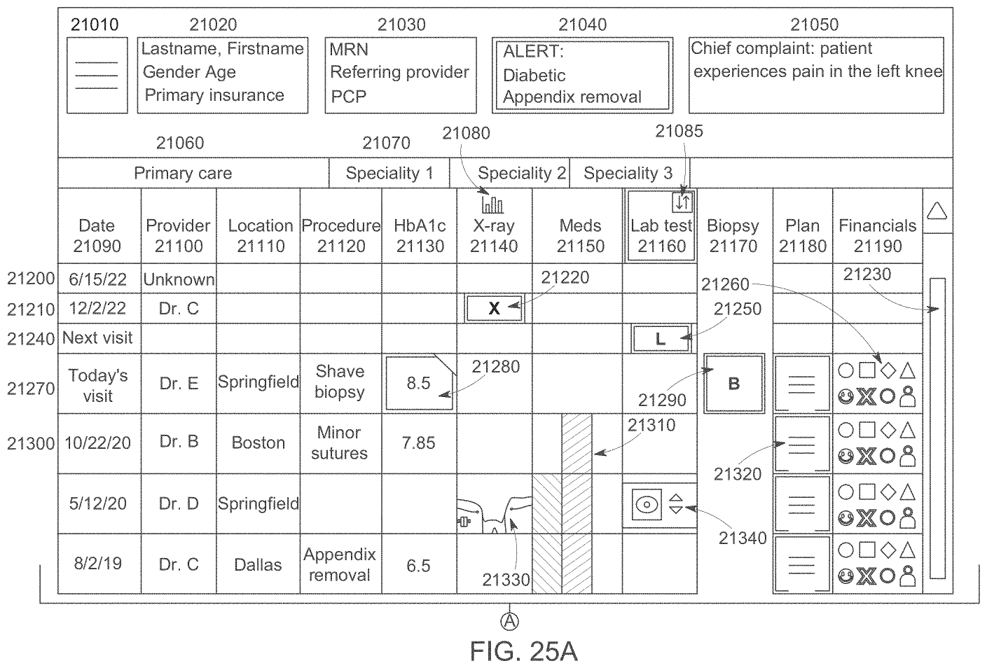

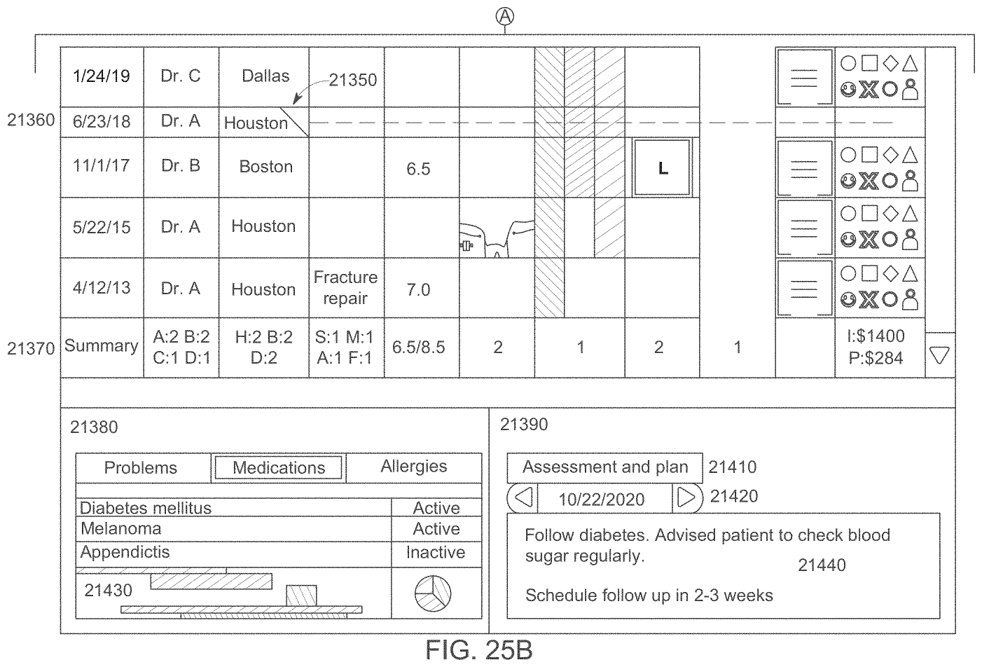

The representation of Dynamic Data of the present principles can take several forms, and exist within several different configurations, as is the nature of dynamic data representation. For example, FIGS. 25A and 25B, (Hereinafter FIG. 25) depicts a Patient-Specific Dashboard display of Dynamic Data in accordance with an embodiment of the present principles. In the embodiment of FIG. 25, a Master Header Row is represented by 21060-21070, a Flowsheet Header Row is represented by 21090-21190, a Flowsheet is represented by 21200-21360, a Flowsheet Summary Row is represented by 21370, and two separate modules are represented by 21380 and 21390.

Embodiments of a Data Command Center of the present principles can be configured for providing a Patient Evaluation Methodology included as, in at least some embodiments, an Electronic Critical Patient Reactivation (e-CPR) technique. That is, embodiments of the present principles can be configured to provide an adaptive, intelligent system for determining which patients are in critical need of care, utilizing a hybrid user-defined/automated Patient Evaluation Methodology, that can automatically take action to rectify identified issues of concern.

For example, in the ever-changing world of healthcare and healthcare IT, sorting through and identifying which data is truly of importance is key to identifying which patients are in the most urgent need of care. In order to accomplish this task, governments have mandated key data elements be recorded, stored, and shared, with the end goal of improved patient care. The downside to recording all of this data is that no human alive is capable of parsing every detail in a timely manner to make the determination as to which patients are of highest concern, and no human is capable of consuming all data points to establish unforeseen patterns of importance. Only through advancements in computing and artificial intelligence can the mass amounts of data be parsed, sorted, prioritized, and acted upon with any level of efficiency. With recent changes requiring the sharing of medical data between EHRs, there still exists no mechanism for alerting doctors or patients as to key factors which may exist in one system, but not within another. No position has ever been defined that requires a person to oversee this interrelationship of medical data, nor would a single person, or even large groups of people, be capable, in an efficient manner, to act upon such vast amounts of information quickly enough to truly manage patient care.

Electronic Critical Patient Reactivation (e-CPR) in accordance with the present principles brings to bear the full power of technological advancement and artificial intelligence to manage a task that existing Patient Reactivation Systems were previously incapable of accomplishing on their own. Through the utilization of established datasets, machine learning, and interoperability, a solution has been realized that can truly accomplish this Herculean task. A system capable of, but not limited to, identifying patients which meet the following criteria, as well as identifying previously unknown criteria, is now possible:
Meet similar Demographic criteria, even with Medical Records in disparate systems or care provided by other providers, that may also meet other key indicators for criticality.
Exhibit Risk Factors, even with Medical Records in disparate systems or care provided by other providers, that may also meet other key indicators for criticality.
Exhibit Conditions, even with Medical Records in disparate systems or care provided by other providers, that may also meet other key indicators for criticality.
Exhibit Critical Conditions, even with Medical Records in disparate systems or care provided by other providers, that may also meet other key indicators for criticality.
Have undergone Procedures, even with Medical Records in disparate systems or care provided by other providers, that may also meet other key indicators for criticality.
Have undergone Critical Procedures, even with Medical Records in disparate systems or care provided by other providers, that may also meet other key indicators for criticality.
Have Diagnostic Test Results, even with Medical Records in disparate systems or care provided by other providers, that may also meet other key indicators for criticality.
Have Critical Diagnostic Test Results, even with Medical Records in disparate systems or care provided by other providers, that may also meet other key indicators for criticality.
Are being seen by specific Specialties or Physicians, even with Medical Records in disparate systems or care provided by other providers, that may also meet other key indicators for criticality.
Have specific Appointment Types, even with Medical Records in disparate systems or care provided by other providers, that may also meet other key indicators for criticality.
Have Future Events Scheduled, even with Medical Records in disparate systems or care provided by other providers, that may also meet other key indicators for criticality.
Have a history of Canceled or Missed Appointments, even with Medical Records in disparate systems or care provided by other providers, that may also meet other key indicators for criticality.

Additionally, with the advent of machine learning, even events or categories of events not previously known can be identified utilizing an algorithm that not only identifies cause and effect but can also deduce cause by effect. In existing patient reactivation algorithms, if key factors are identified beforehand, patients may be identified which meet the criteria, e-CPR New Category Identification accounts for, but is not limited to, evaluating:
Pre-Identified Factors
Existing Data
Historical Event Factors
Current Event Factors
Future Scheduled Event Factors
Key Events
Key Results

In each of the areas described above, the evaluation is not limited to a linear parsing of data. Each step may be accounted for, but e-CPR utilizes machine learning to identify when patterns exist that were not previously identified, and automatically begins accounting for the newly acquired data. As an example, a doctor or patient reactivation system can know that a patient with diabetes needs to be seen every 1-2 years, and a patient with diabetes and heart disease may need to be seen every 6-12 months, but can be completely unaware that patients within 50 miles of a specific zip code are exhibiting complications requiring them to be seen every 3-6 months. The complications may not have yet been identified or correlated to this location, but utilizing advanced pattern analysis, e-CPR can parse all patient records and identify a pattern of worsening outcomes linked by locality. The root cause can be a factory with faulty filtration, environmental conditions, or even a localized outbreak. Such contributing factors would not have been identifiable without first connecting the data that patients with specific conditions have worsening outcomes, and that the patients exist within a certain locality. With the local population seeking healthcare at several different facilities, each facility may not notice the increased pathology due to the small sample size and focus on individual patient care. Only through correlating several factors from all locations in the area do such issues come to light.

In another example, a specific patient can have a history of relatively minor risk factors, conditions, and/or procedures, but poor compliance with maintaining a proper schedule of doctor visits. Such a patient can see 4-5 different providers, all at different locations, or can only visit a hospital for emergency care when conditions become unbearable. No one provider may be aware of the history of missed appointments because they are only missing a few appointments at each provider. In such an example, the reason for the missed appointments now becomes a higher priority. If the patient is missing key appointments with specialists they have been referred to see, such a patient is in danger of becoming critical. As mentioned, this can lead the patient to visit the emergency room for conditions that would have best been addressed through routine care. Identifying such a patient is critical, not just for individual patient care, but also for determining factors to identify similar patients before they reach this critical state, such as socio-economic conditions, language barriers, lack of transportation, and the like.

Identification is only the first step in electronic Critical Patient Reactivation. After proper identification, the most important factor is ensuring patient compliance. Existing patient reactivation systems implement many established means of communication to send a reminder to the patient to come in, by mail, email, text, and/or automated or manual phone call. In some more advanced patient reactivation systems, responses to communications may be tracked and accounted for, such as a missed phone call may be followed up on x more times, and a report can even be generated to show non-compliant patients. With e-CPR's advanced communication management, method of communication with a given patient is not limited to patient or practice preference. Similar to identifying critical patients for reactivation, an algorithm is used to determine the most effective means of communication. Historical data is compiled and analyzed, not just for the individual patient, but also correlated against other patient with similar age, conditions, locality, and other key factors, to determine that a certain patient can prefer a text message between the hours of 8 AM and 5 PM Monday through Friday, a cellular phone call between 5 and 6 PM on the same days, and 10 AM through 8 PM on weekend, but desires a home phone call outside of those times. Historical data may also point to a dramatic inability to contact a patient through established methods, thus determining a certified letter or even an in-person visit may be required to ensure said notification is delivered and received. By compiling and correlating all available data, e-CPR's AI can determine not only the most effective means of communication, but the most effective times to communicate via a specific method.

e-CPR is not limited to a single action or set of actions in response to an attempt to contact and bring a patient back in. Several steps may be predefined, but, as is the strength of e-CPR, additional steps may be defined or redefined based on current or future responses. E-CPR is also not limited to patient communication in an attempt to reactivate a critical patient. With connections throughout the care process, e-CPR has the ability to send tasks to schedulers, visually notify doctors at point of care, and even reach out directly to all of the patient's doctors, to immediately make them aware of the need for the patient to be seen for a specified reason. A process of the present principles can implement several conditions, steps, requirements, and triggers to ensure that the patient is never lost within the system. FIG. 13 depicts a Table having example parameters of a process for e-CPR in accordance with the present principles.

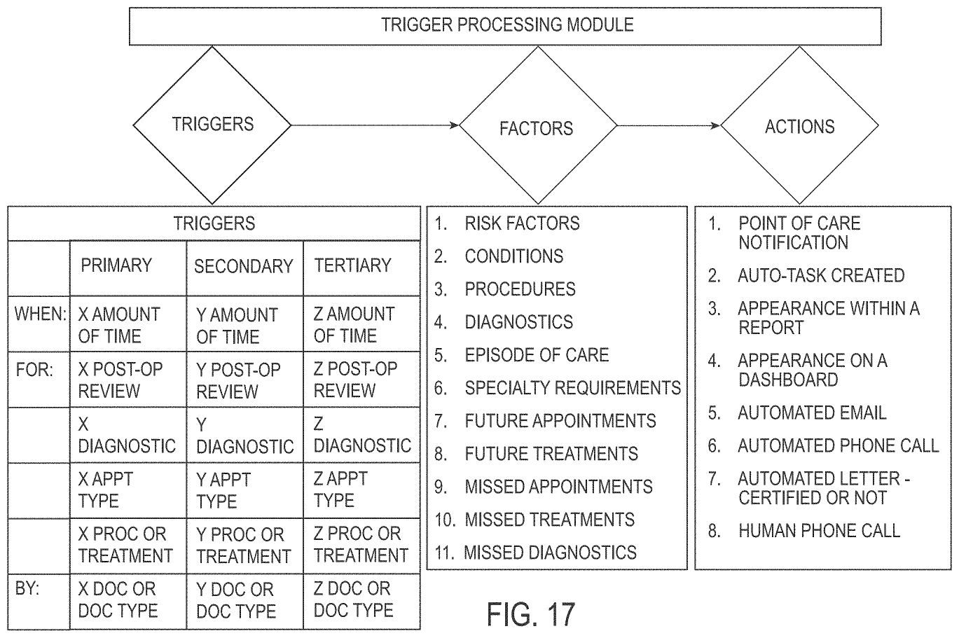

An example of such a process is depicted in FIG. 17. While the example of FIG. 17 depicts a total of three triggers, triggers are not limited by number, nor are the steps or information contained within meant to express a limitation or linear process. Multiple and complex arrangements of triggers, counter-triggers, and multi-dimensional triggers can be implemented as needed to accomplish the requirements of electronic Critical Patient Reactivation of the present principles. It should also be noted that, in FIG. 17, the Y-Axis display listing When, For, and By, is not limited to these values or this number of values. Predefined criteria, as well as criteria determined at time of execution, can add, remove, or otherwise alter this criteria list.

In the process of FIG. 17, actions to be taken can include, but are not limited to:
Point of care notification to a Provider
Auto-Task created to a Scheduler or Doctor with enough information to make a medically relevant decision
Appearance within a report with enough information to make a medically relevant decision
Appearance on a dashboard
Automated email to Patient, Practice, Provider, Scheduler
Automated phone call to Patient, Practice, Provider, Scheduler
Automate letter, Certified or not, to Patient, Practice, Provider, Scheduler
Human phone call to Patient, Practice, Provider, Scheduler
Human visit to Patient, Practice, Provider, Scheduler

As noted above, in embodiments of the present principles several key factors are compared and can affect the outcome of the critical patient reactivation workflow, either by triggering an action, or satisfying a requirement. Triggering an action occurs when a requirement is met for a trigger. For example, if a requirement for a patient with specified risk factors and conditions is not met, as in an obese diabetic patient with glaucoma not receiving a diagnostic test to track their condition within 6 months, and action may trigger a point of care notification to the specialist tracking the disease. If the requirement is not met, and a second threshold occurs, such as not receiving the diagnostic test in 18 months, an auto-task and alert on a dashboard can trigger to the schedulers to ensure the patient is scheduled for the diagnostic. A third requirement can occur if the first and second requirements are not met, which can trigger a series of notifications to the assigned specialist, and possibly all users providing care for the patient, the schedulers, and the patient, to ensure the patient is receiving proper care.

In accordance with the present principles, should additional data become available during the course of the above described process, such as a new procedure or condition being recorded, the algorithm can be reinitiated or refactored as required based on the newly acquired information. Should a patient commit to a future event, such as scheduling an appointment, which satisfies existing criteria, an additional step can be created to ensure the future commitment is met. If it is not, the patient can reenter the previous workflow, or newly defined requirements can be established.

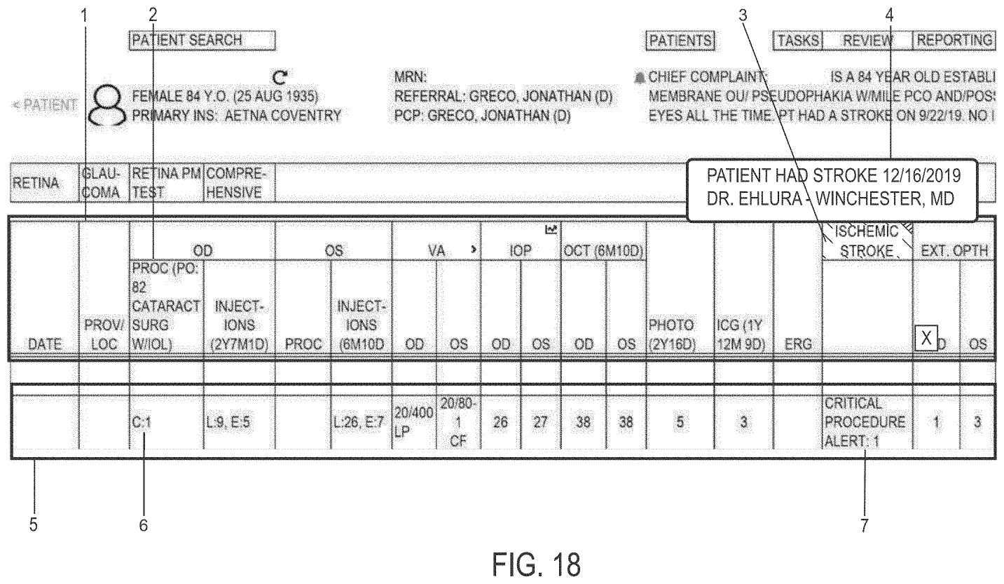

Critical Patient Indicators of the present principles can directly interact with Dynamic Health Records. For example, FIG. 17 depicts a flow diagram of a trigger processing method in accordance with an embodiment of the present principles. As illustrated in FIG. 18, at element 1, the Dynamic Health Records Header Row is displayed. Such a row denotes column names as well as key indicators regarding the contents of said column. At element 2, an indicator for a Cataract Surgery Post-Operative Period is clearly denoted. This indicator states that the patient is in the 82nd day of a 90-day Post-Operative Period. As such, an Ophthalmologist would clearly recognize the criticality of said patient. A column which would not normally be displayed, element 3, Ischemic Stroke, is automatically added to the Ophthalmologist's dashboard by way of Patient Evaluation Methodology, to show that said patient has had a Critical Procedure. By way of hovering over, selecting, or otherwise interacting with said column, more information may be displayed as shown in element 4, denoting the procedure, date, physician, and location where the stroke was recorded.

In FIG. 18, element 5 denotes a Dynamic Health Records Summary Row. Such a row denotes counts, totals, min/max, assessments, best/worse, et al values based on the contents of said column. At element 6, a Count of procedures is displayed, in this case, C: 1 to denote one cataract procedure has been performed in the right eye. Finally, at element 7, a summary for the newly added column, Ischemic Stroke, is displayed denoting that a Critical Procedure Alert exists in the column. This is an example of the display of a Point of Care notification to a Provider.

Indicators can also exist within columns, within rows, outside of rows or columns, attached to modules, panels, in popups, pop outs, pop overs, and by other methods used to notify a provider. Indicators are not limited to visual indicators and may employ sound, voice recordings, and other audio methods of notification. Indicators may also include vibrational feedback and other means of notification available based on the media used to access e-CPR and/or Dynamic Health Records.

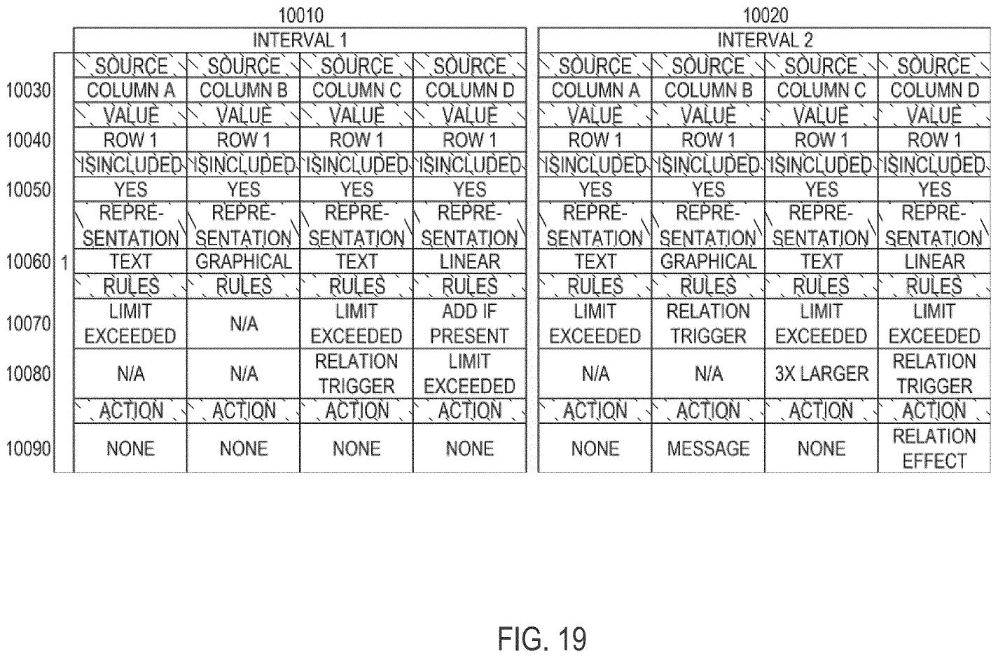

FIG. 19 illustrates a series of Data Visualization Storage configurations in accordance with an embodiment of the present principles. In the embodiment of FIG. 19, data visualization is achieved with a series of configurations to determine what and how to display. In some embodiments, Source Data (10010) consists of at least one of a Value (10030), of at least one of an Inclusion/Exclusion Rule (10050), of at least one of a Visual Representation Configuration (10060), of at least one of a set of governing Rules (10070, 10080), and of at least one of a set of Actions to be taken (10090). The data can be visualized across multiple intervals (10010, 10020).

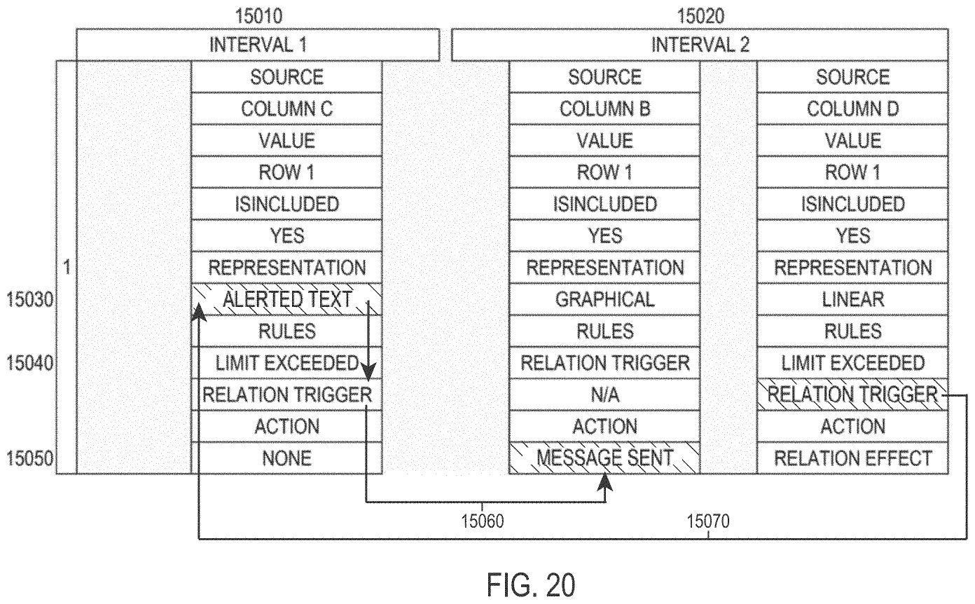

FIG. 20 illustrates interactions between data sets in accordance with an embodiment of the present principles. In the embodiment of FIG. 20, data stored or generated at runtime can be correlated and evaluated against other data stored or generated at runtime, reinitiating the process until no further changes occur. In such a process, data recorded for interval 1, as illustrated in 15010 of FIG. 20, can be correlated and evaluated against data recorded for interval 2 (15020). At 15030, Alerted Text in column C is displayed, which was previously listed as Text in column C 10050 of FIG. 19. This can be triggered by a series of actions occurring, such as a Limit Exceeded Rule invoking a Relation Trigger 15040. Such a Relation Trigger directly affects 15070 the field specified in the Relation Trigger, in this case, reevaluation of the Representation in Column C 15030 of Interval 1 15010. Subsequently, the text of Column C 15030, now invokes a Relation Trigger 15040 of its own, which directly affects 15060 the field specified in the Relation Trigger, in this case, reevaluation of the rules for Column B of Interval 2 15020. As such, a Message is now sent.

Configurations stored can be correlated between intervals, within the same interval, from different source data, and between separate categorizations of visualized data. Utilizing the methodology, one can assume any change in source data may initiate refactoring of the evaluation process, as well as any change, addition, or deletion in displayed data can initiate refactoring of the evaluation process. Furthermore, data added for future consideration, such as future appointments or orders, can be evaluated, displayed, and reevaluated.

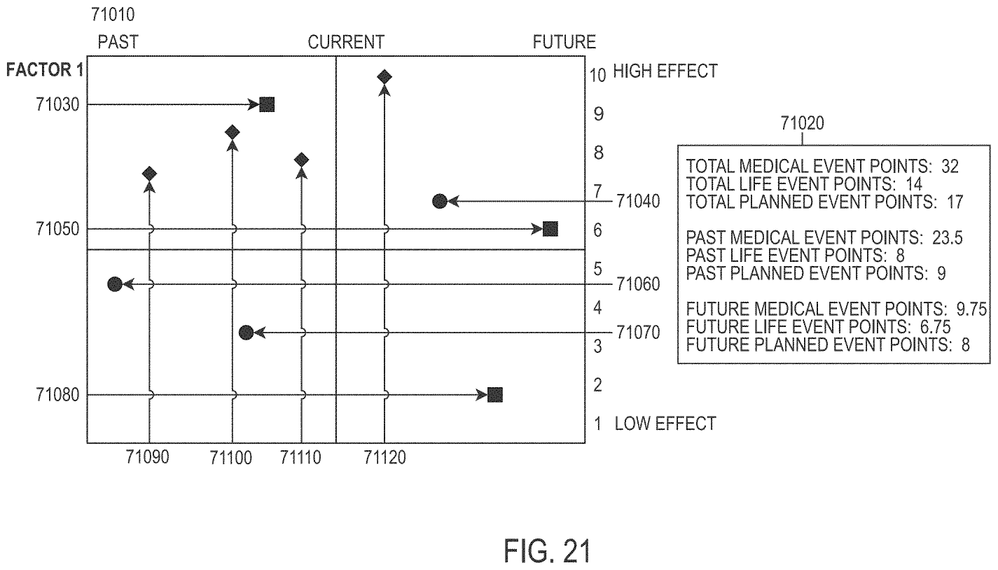

FIG. 21 illustrates a weighting system in accordance with an embodiment of the present principles. In FIG. 21, 71010 displays a correlation between Effect (importance) and Occurrence (represented in time). 71020 illustrates an overall total of different categories of events, as well as past and future event totals. In the embodiment of FIG. 21, weighting can occur at an overall level with a full Whole Life View displayed, or in past or present as required by zoom level, and weighting can change based on relevance of events to other events. In this example, a planned event is displayed at 71030. The planned event can be representative of a planned appointment or treatment that was not met, or the planned event can be representative of a medical event. At 71050 and 71080 future planned events are displays. Future planned events could be representative of appointments or orders scheduled for future dates. Past, present, and future planned events can interact such that a missed planned event 71030 can increase the importance of a future planned event, or medical or life events.

In FIG. 21, at 71040, a view for a future life event is generated. This could be representative of events such as a wedding or important anniversary, which can have an effect on the weight of prior or subsequent events. 71060 and 71070 represent past life events, such as a divorce or loss of a loved one. Life events can affect the overall health and compliance of a patient, and as such are weighted and accounted for. In FIG. 21, 71090-71110 represent past medical events, procedures, injections, surgeries, diagnostic tests, or any other medical event in the past. Each medical event is weighted and correlated with each other and other events. 71120 represents a future medical event, not a planned event, because a medical professional may know, with increased certainty, of the occurrence of this future event. Such an event can include a transplant or other major procedure for which there is little to no option for the event to not occur.

With such representations of weighting 71010 in accordance with the present principles, an axis for effect ranging from low to high, as well as past to future is enabled. This is only one example of a correlation, although many correlations work together for ultimate determination of the weight, as mentioned by the zoom level refactoring the weight of events. Total event points 71020 rise and fall based on such factors, until a final result for the specified view is determined. Results can be refactored based on changes to source data or actions taken to alter displayed data.

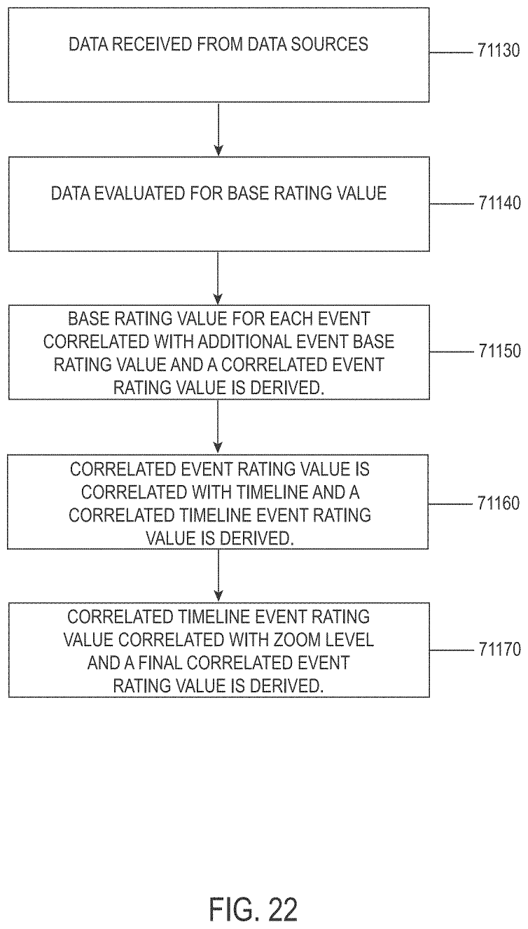

FIG. 22 illustrates a flow diagram of a weighting method in accordance with an embodiment of the present principles. In the embodiment of FIG. 22, data is received from data source 71130, either external 10010-10110 of FIG. 3, internal from application storage 10160-10180 of FIG. 3 or the Rules Engine 10210 of FIG. 3. Initial data evaluation occurs, and a Base Rating Value is assigned based on type and contents of data received 71140. Data evaluation ratings can be stored. Once Base Rating Values are assigned to events, all events are evaluated against each other to determine a Correlated Event Rating Value 71150. This Correlated Event Rating Value accounts for the interactions of key events and can weight one event higher or lower based on the existence of another event. Once all events are evaluated and Correlated Event Rating Values are assigned, each is then reevaluated based on when the event occurred, resulting in a Timeline Event Rating Value 71160. At each step, resulting values can be stored in volatile memory until no longer needed. Once all evaluations of and between data and timeline occur, the view is then accounted for. At 71170, the specified zoom level is factored in, and a Final Correlated Event Rating Value is used to determine the final weight of the values to display.

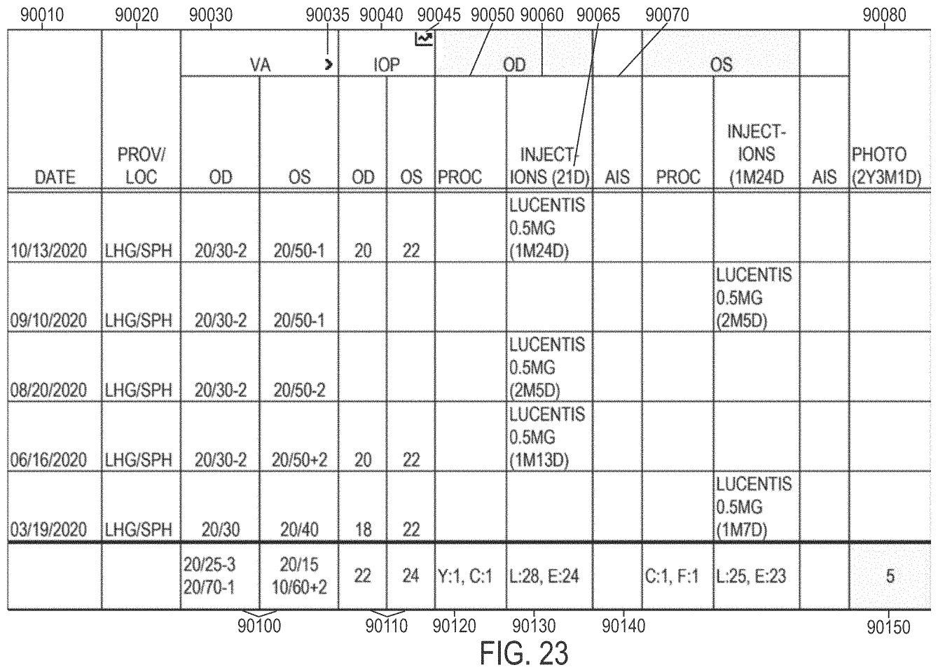

FIG. 23 depicts a Flowsheet depicting Header and Summary Row functionality as it pertains to Dynamic Data Sets in accordance with an embodiment of the present principles. A key feature of the Data Command Center is the ability for Flowsheet Header and Summary data representations to be programmed, or intelligently adapted based on rules defined in the Rules Engine 10180 of, for example, FIG. 3, to display notifications and allow interaction with the data that lies between them as describe in Summary Data Representation 20140 of FIG. 14. In the embodiment of FIG. 23, the Flowsheet Header consists of a Date 90010, Doctor and Location 90020, Visual Acuity 90030, Intraocular Pressure 90040, Procedures 90050, Injections 90060, Medications 90070, and an array of Diagnostic Tests 90080. Each header can behave differently based on the data contained within the represented data set, data contained within other data sets, data displayed, and/or data not displayed.

For the purposes of the embodiment of FIG. 23, Date 90010 is the date of an appointment, encounter, occurrence, or event which pertains to the treatment and care of a patient. Doctor and Location 90020 is comprised of any doctor or caregiver, and the location at which they interacted with the patient. Also displayable within this data set may be diagnostic testing equipment, equipment which can exist outside of the practice such as remote monitoring systems or take-home testing equipment, and/or anything that may provide a result or measurement which pertains to patient healthcare.

In the embodiment of FIG. 23, Visual Acuity 90030 depicts a summary of multiple Visual Acuity test results. Within the VA header, there are OD and OS column headers to accurately display the Right Eye (OD) and Left Eye (OS), yet any column can be set to a single group, multiple subgroups, and the subgroups can also contain multiple subgroups. At 90035, an expander is available within the header which will expand to show several data sets with distinct Visual Acuity results based on a number of methods for testing. It should be noted that the expander can be implemented to depict, or in the inverse, to hide, a number of data sets. The Expander may be set to Expanded—True, and all data sets will show unless the icon is selected to collapse them. The Expander can also be set to Expanded—False, in which case, all specified data sets will be collapsed unless the icon is selected to expand them. As seen in this example, one data set, VA, is set to expanded, while several other related data sets are set to collapsed. In another embodiment, the Expander can expand to show all or collapse to hide all, or any combination of showing and hiding data sets as denoted in configuration.

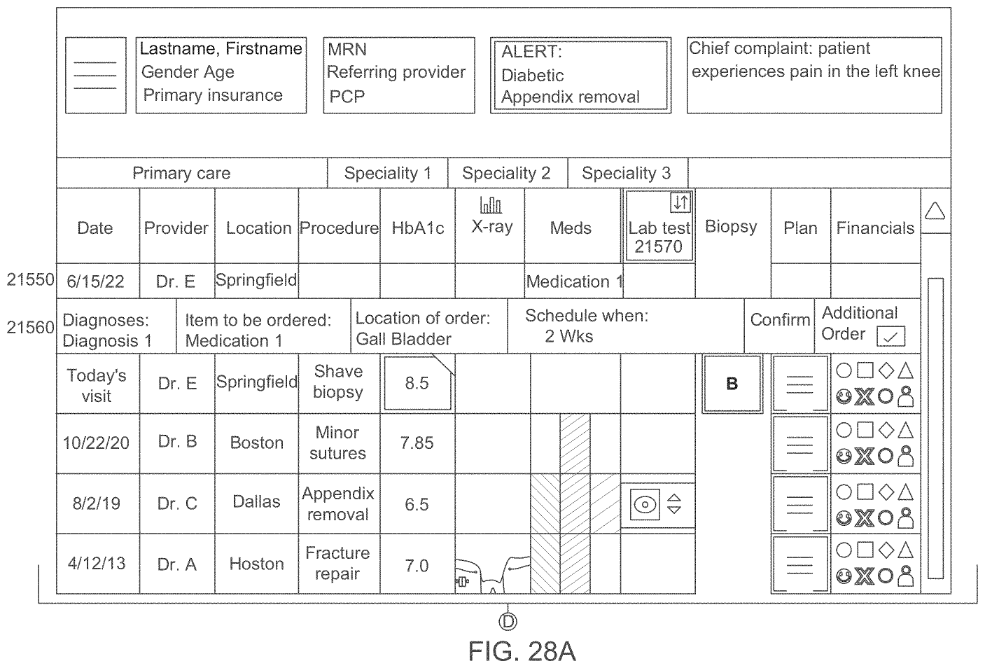

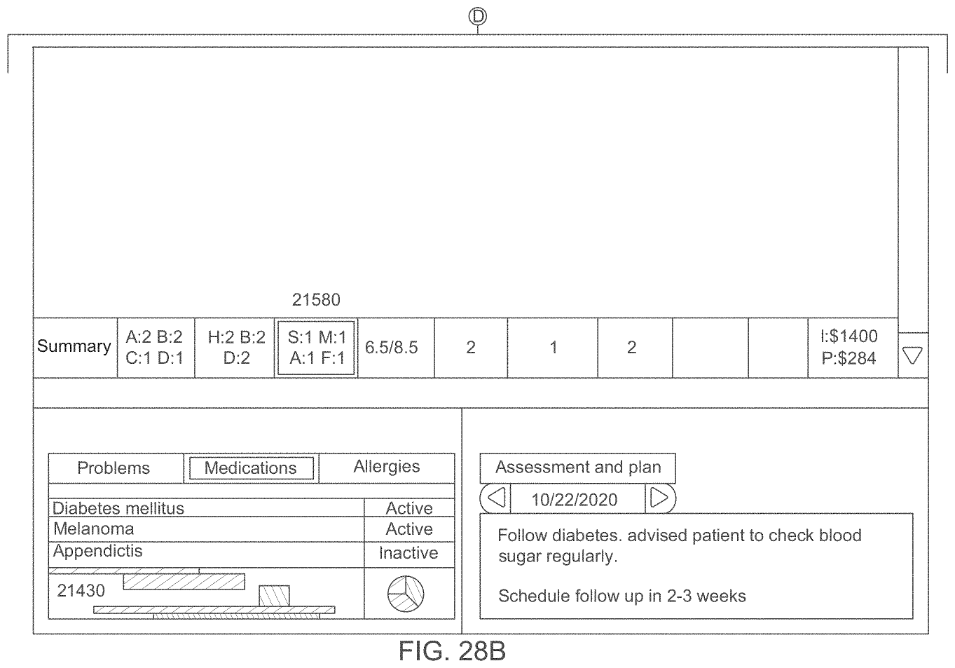

In the embodiment of FIG. 23, VA 90030 also contains a summary field 90100 denoting the best and worst values within the data set. Best VA is denoted in a first color (e.g., green), while Worst VA is denoted in second color (e.g., red). It should be noted that a series of configurations can be set to determine color, size, position, and purpose of any summary row. Summary Row data fields are interactive. In the summary field 90100, selecting the Best or Worst value may collapse all other fields to show only the value selected, as demonstrated in FIGS. 28A and 28B, (Hereinafter FIG. 28). Fields can be expanded to show when a user deselects the summary field value.

Intraocular Pressure 90040 contains two sub data sets, OD and OS, to represent the right and left eye. Within the parent group, results of TOP testing are displayed. At 90045, one will note an icon of a graph. The icon can initiate a correlative graph of several key factors, including but not limited to, Date, TOP, Medications, and Procedures. An icon, such as in 21080 of FIG. 25, can exist anywhere within a header, and on any portion of the application, to launch a different representation of the data.

In the embodiment of FIG. 23, TOP 90040 contains a Summary Data Representation 90110 displaying the highest recorded value in the column. It should be appreciated that the field has been configured differently than the VA summary field, and a series of configurations can be set to determine color, size, position, and purpose of any summary data representation.

In FIG. 23, Procedures 90050 can display any or all medical procedures performed on a patient. Procedures has a summary field 90120 which denotes a count of each type of procedure which was performed on a patient. Injections 90060 is a segregated group of procedures. It should be noted that any portion of the Data Command Center of the present principles can be configured to display any record, group of records, and calculations based on records. At 90065, a Header denotes the number of days since the last occurrence of an event in that data set. As with Procedures 90050, Injections 90060 also has a summary field 90130 showing the total count for each type of injection procedure within that data set.

In the embodiment of FIG. 23, Medications 90700 display in a graphical data representation. A single bar exists in the example because the patient is only on one medication. All other medications are hidden in accordance with dynamic data representation describe herein. A medication, in this example, displays as a bar beneath said header, originating at the start date and concluding at the end date. The summary field, unlike in previous examples, will display as many events as the medication takes up from the start date through the end date. All events which do not contain an instance of the medication bar will be collapsed until such time as the user deselects the summary field.

In 90080, a data set containing Fundus Photos displays a header denoting the amount of time that has elapsed since the last Fundus Photo occurred. At 2 years 3 months and 1 day, an alert is triggered by a rule which states the diagnostic test should occur within a specified timeframe, highlighting or otherwise emphasizing the header field in a different color. A corner informational data representation in the header allows for the user to view the precise reason for the alert. As no Fundus Photos have been performed in over 2 years, and with the display only showing the current year, the importance of the summary field 90140 becomes apparent. The summary field denotes a total of 5 Fundus Photos have occurred within the time of patient care, and the column header denotes there has been over 2 years since the diagnostic was performed. In FIG. 23, Summary fields 90100 through 90150 act and interact differently with the displayed data. Each serves a purpose, and while the purpose can differ, it should be noted that singularly, or in conjunction, all summary fields can be used to alter the display of data, and return said display to its original state.

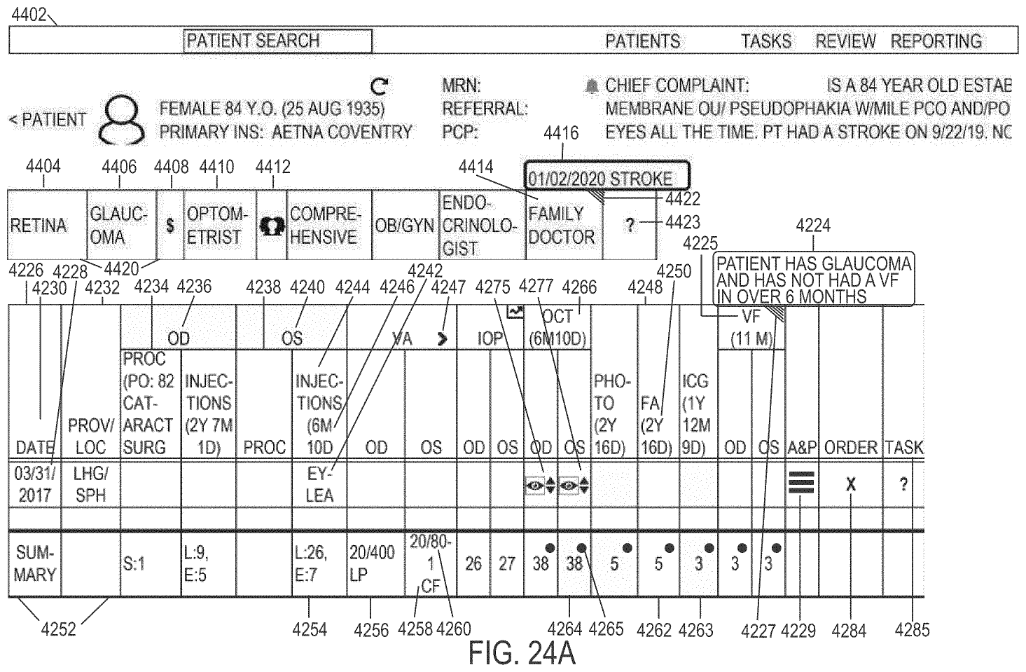

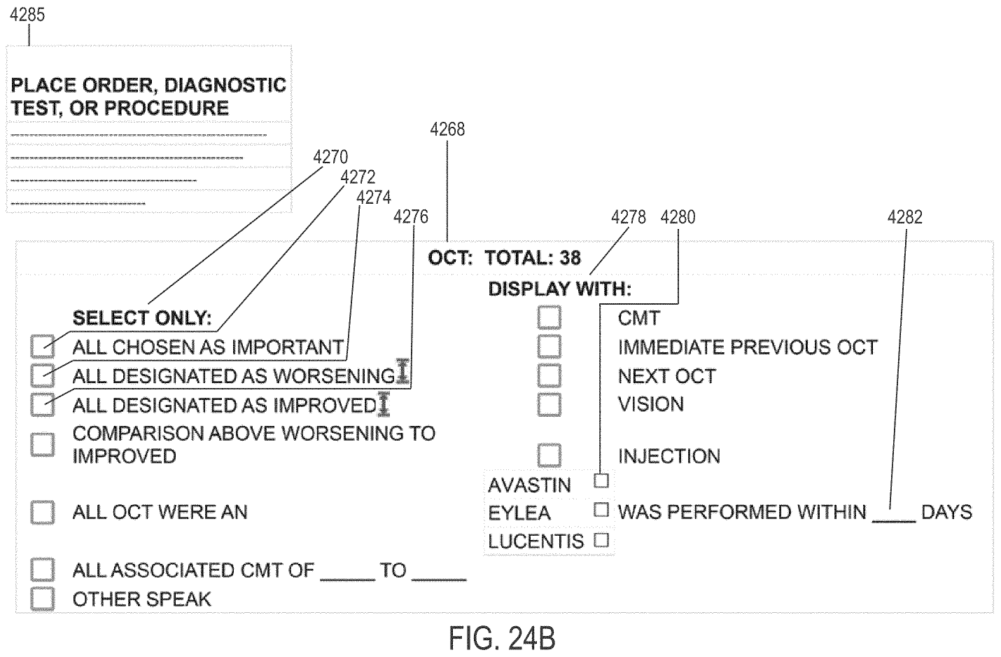

FIGS. 24A and 24B, (Hereinafter FIG. 24) depict an alternate embodiment of a Flowsheet depicting Header and Summary Row functionality as it pertains to Dynamic Data Sets in accordance with an embodiment of the present principles. The embodiment of FIG. 24 depicts the functionality of three specific rows in the user interface in accordance with an embodiment of the present principles. That is, FIG. 24 illustrates how three rows or panels in the user interface 4218 can convey a plethora of information for a healthcare professional in some embodiments. Header 4226 is an example of a header that can appear on each individualized specialty-based provider's actionable dashboard. As illustrated at 4220, different actionable dashboards that have been particularly designed for different providers of different specialties can be accessed. In the embodiment of FIG. 24, a summary row 4252 can be provided on each individualized dashboard for each doctor, specialist, or other user and can be specialized for each user. In the header 4226, the date is represented at 4230. The date can include a time, day, and/or date of a patient visit or the visit of a group of patients. 4232 can include the initials or name of the provider who cared for the patient or if just a location of testing, can include an indication of the location of the test performed. That is, in some embodiments, a medical care provider's initials can be presented at 232, which can also include a location, as providers can have multiple offices. In some embodiments, the information in 4232 can include an abbreviation, description, or identifying factor of which office a patient visited. In FIG. 24, 4228 shows an example of one of many patient encounters over time.

In FIG. 24, 4236 depicts an example of a column that includes a procedure and, in the embodiment of FIG. 24, is divided into two different sides of a patient's body. In 4236 OD is displayed, which in eye care refers to a patient's right eye. In the case of orthopedics, 4236 could reference a patient's right knee. Similarly, an OS in 4240 represents the left column or in the case of eye care, the left eye, and in the case of orthopedics can refer to a left knee. As illustrated, the OS or left column of the header can be a procedure 4238. Under the column of the left eye is listed, by encounter, identifying procedures such as injections 4244. Item 4242 depicts an injection that has been performed (e.g., injection of Eylea is listed but can represent any data related to the column and row it is under.), while 4228 shows the date of the medical encounter which by way of example shows Mar. 21, 2017.

As illustrated at 4234, the header 4226 is able to display that a cataract surgery has been performed and that a postoperative period is counting down. The header 4226 can also display that injections 4246 were last performed six months and ten days ago. 4224 can display a last time this test or item was performed on a patient or any kind of alert for instance if patient is allergic to the item or a condition impacts this test or procedure and 4224 can be on any column or row. Cell 4225 can have any highlights or otherwise emphasis that can inform additional information such as an alert that something has not been done but should be in one year, such as orange, or if not done in two years that can be a severe warning so cell could be red. 4227 is a mechanism for user to learn more details and more information can come up for example 4227. In the embodiment of FIG. 24, 4224 depicts that a test was performed 2 years and 16 days ago. Header 4226 also includes a pop-up 4224 of underlying information. In the embodiment of FIG. 24, the patient has a diagnosis of glaucoma and has not had a visual field in over six months. 4247 shows an expanding mechanism where many more columns can suddenly appear. In this case, it appears in VA (vision) cell and there are many ways to test a vision, which can have different data. To save space on the display, the other methods are hidden until the user clicks on 4247 and then other columns are displayed. Clicking again on 4247 reverses the process and the columns are hidden again.

The summary row 4252 of FIG. 24 depicts how not only is the total number of something that had occurred in the rows above counted, but it can be divided according to what was performed. That is, in the embodiment of FIG. 24, summary row 4252 is a smart summary column. In the embodiment of FIG. 24, 4254 demonstrates that there were twenty-six Ls and seven Es of which one E (Eylea) is shown at 4242. The embodiment of FIG. 24 depicts an example of a retina doctor who performs injections in the right eye in this case, and used “L,” which stands for Lucentis times and “E,” which stands for Eylea. 4256 similarly demonstrates the summary cell in a column of the left eye. 4260 shows the vision in the left eye is 20/80-1 and could also reflect the best number or event that occurred in the entire row overall of dates of service, and can be highlighted or otherwise emphasized to inform the user that it is the best value. 4258 shows CF. In this illustration of a retina surgeon, CF means count fingers, which is very bad vision and is, therefore, red. For the first time in this illustration, a retina surgeon can know, over the time that the doctor has been delivering services, or any doctor, what is being reflected in encounters and rows above. The best vision was 20/80 (4260). The worst was count fingers (4258). This can also be the best blood pressure and the worst blood pressure. Every different specialty in medicine has different ways that it would like to measure the highs and lows in a column. 4262 simply shows the counting of the number that had occurred in all of the encounters. In this case five FAs 4248. Patients may be seen over a hundred times and many medical services are provided. 4252 can be implemented to first show the doctor summary and identify critical items for the doctor to consider and for the first time the user can instantly view whatever is critical. In some embodiments, the user simply clicks on any of the information in 4252 and instantly all the encounters related to that data clicked is displayed in the number of rows 4228 that are needed to show the data. If a user engages by any method 4262, five rows of 4248 (in this case FA), are displayed in 4228 with five rows of 4228, since there are five listed in 4262. If user also wants to view simultaneously 4263 (3 ICG listed), then three additional 4228 will be displayed. So, if 4262 and 4263 are clicked, a total of eight rows of 4228 will be revealed unless some of those two (2) tests were done at the same visit. Clicking again on 4262 and 4263 can reverse the process. 4202 provides another way for the user to sort information and display in 7126.

It is important to note that the Data Command Center of the present principles can measure anything in the row and display it in multiple different ways. The choice could be just to see the high and low as in 4260 and 4258 over a short period of one year or over as many years as there have been encounters. It can also be set to show percentage changes over time. In any case, this summary provides a tremendous amount of information to the provider for enabling rapid decisions.

A panel 4220 can be located at the top, side, or bottom of the display and can provide access for each specialist to different types of healthcare providers or different doctors who want to customize the display. Any type of doctor or dentist or other health care provider of any specialty can be listed. As few or as many as have actionable dashboards that can be accessed immediately with direct access by simply clicking on the specialist's name. For instance, the specialists can be retina 4220, glaucoma doctors 4206, or an optometrist 4210. All three happen to be types of eye doctors. All three could be in the same practice, separate practices, or even in different countries. Each, when clicked on, pulls up an actionable dashboard specially designed for them or their practice in that specialty. 4214 provides an example of a non-eye doctor, in this case, the family doctor.

It is important to note that any health care provider, if given permission by the patient, and each specialty noted in FIG. 24 could see the actionable dashboard of the other specialists for as little or as much as each would allow. There is some information in an actionable dashboard to each specialist, practice, or doctor that they might not want others to see, which can be hidden (e.g., payments and costs). In addition, next to each actionable dashboard can also be additional information that can also be pulled up instead of the actionable dashboard itself. For instance, a dollar sign 4208 could be for providing for each practice or actionable dashboard, payments, costs, or any financial matter that can pop-up to show a different type of financial dashboard. 4212 shows an example that can pull up any type of additional information, such as a shared care dashboard between different providers.

4222 illustrates that an entire cell can alert all of the other providers of something important. It can be a color change, or flash, or blink. When activated, it represents that there is some type of important event, for instance, that all providers should know. A pop-up 4216 also can be shown at all times or by hovering over 4222. The popup could represent whatever the important item is to be alerted, for instance, a new diagnosis like that the patient had a stroke on Jan. 2, 2020, which all providers would like to know. It can also inform all providers that the patient missed an appointment that was particularly important with that doctor. So, that all specialists would know that and be able to remind the patient. The critical information in 4216 can also be inputted by creating a row 4228 in time order or as the first row that every provider views when they open their personalized flowsheet.

It will be further appreciated that the actionable dashboard can further include a communication center where users can send messages to each other in a HIPAA compliant way. A physician, while seeing a patient, can send a message in one of many ways for instance, by clicking 4223 and a mechanism to send a message to any other doctors caring for the patient, even if not in their own practice but another practice such as 4214 or 4210 and a message sent and added to that patient's actionable dashboard in the other practice. This mechanism can also set an alert, as seen in 4216 or allow any doctor when they believe something is so important for all providers to know to set a row in all providers tools and creating a row inserted. Doctors can also send messages within their own practice such as to their chief technician or the office manager to talk about following up on a patient or also to the billing office that there is a billing problem. Then, staff can report back to the doctor and this message can be imbedded into the smart actionable dashboard so that the next time a doctor sees the patient through icons and columns of correspondence of communication within the practice, the doctor can pull up what was the response to a message they had sent earlier. This response can be read live while treating the patient so that the doctor can take it into perspective while making decisions. The messaging system, attachments, or anything else can be sent to the doctor or health care provider in any way that they would want. Whether through email or the internal messaging system or as a tickler system within the EMR system that automatically toggles back and forth to the actionable dashboard, so the doctors can see their messages at the end of the day or the end of the week, or while seeing the patient. It really helps organize the doctor's life, so this actionable dashboard becomes the communication hub, the switchboard, for the entire practice, while communicating with the health care provider.

FIG. 24 further depicts an embodiment of how the interaction with a header and summary row can work. When a doctor interacts with the Flowsheet of the embodiment of FIG. 24 and element number 4252 and clicks on the numbers. In another embodiment, colored dots can be depicted to enable a doctor to refine further exactly what they would want to see. Clicking on 4264 for instance on the summary row Element 4252 brings up just those encounters when that particular test was done and the 38 OCTs performed would come up and be displayed in 4228 where one such OCT encounter is depicted. If the user wants to sort further, then in one embodiment an example would be clicking on a colored dot in 4265 which enables a user to refine exactly what data the user needs to visualize to make decisions. For instance, upon activation a display panel can display on the screen when 4265 is activated. As such, a user can select 4270 and now many options can be selected to precisely sort and search what the user wants to find from the data. For instance, the user can choose all OCT's 4272 or just choose the OCTs that show worsening with the colored (e.g., red) arrows, 4274 which would select diagnostic tests with colored identification 4275 which could mean many things such as diagnostic tests with worsening result. The user can select to see only tests that are as designated as improved perhaps in this case as another colored (e.g., green) icon 4276. Only the OCTs that have that designation and the time of those encounters would be displayed. As can be seen, many different options on exactly how to refine which OCT can be selected.

In addition, other encounters can be selected to also be displayed along with the initial data encounters selected in 4270 of FIG. 24. Patients can have hundreds of visits and if a user wants to refine what they're looking for, the user can choose and pick out any particular diagnostic tests that share a common features as seen on FIG. 24, 4268, and the user can ask for other encounter dates and rows to be also sorted and displayed such as also showing if injections of Avastin, 4280, were performed within a certain time period 4282 of the OCT's, where criteria was selected to be displayed in 4270.

In FIG. 24, 4266 can be implemented to bring up an ordering panel 4285 or to expand other areas for ordering but always though one user interface and one display. Now the user is enabled to place an order while visualizing relevant data.

The Patient-Specific Dashboard of FIG. 25 can be accessed by direct login to an application, launched from a separate application, either within the context of an existing patient from the application or generally through a patient search, or from within other aspects of a Command Center. A Master Header, in this example, is used to represent general data and information about a patient. A Navigation icon 21010 can be utilized to exit the Patient-Specific Dashboard, navigate to other aspect of a Command Center, or return to an initiating application. Patient Demographic and Insurance information can be represented in 21020. Medical Record Number and Associated Providers can be represented in 21030. Critical Patient Alerts can be represented in 21040, highlighted or otherwise emphasized, such as in color, to draw attention to them. The Chief Complaint, or purpose for a visit, can be represented in 21050. In the embodiment of FIG. 25, the key purpose of a Master Header is to denote the context within which all subsequent data representations exist.

The Master Header Row 21060-21070 can be utilized to differentiate different personas. In the embodiment of FIG. 25, Primary Care 21060 is the selected Persona, and as such, data is represented in accordance with requirements of and for the Primary Care Persona. Beyond this persona, distinctions can be made per user or per other configurations, but the general governing persona remains Primary Care. Other Personas, such as Specialty 1, Specialty 2, and Specialty 3, collectively 21070, can be accessed through their respective fields, and can be highlighted or otherwise emphasized to draw attention to important aspects of patient information which may not be represented within the selected Persona's view, such as Specialty 3 being highlighted. Personas can include, but are not limited to, user- or specialty-defined personas, practice defined personas, or any logical or geographical representation, not limited to a single user or practice, and inclusive of outside practice patient information.

The Flowsheet Header Row 21090-21190 denotes, in this example, column headers. Said Flowsheet Header Row is not limited to columnar data, and may exist vertically, or along any axis, or exist outside the context of an array of data. The Flowsheet Header Row, in this example, is comprised of Summary Data Representations 20140 of FIG. 14, accounting for all data in the underlying column or logical grouping to be represented by Summary Data Representations.

In the embodiment of FIG. 25, the Flowsheet Header Row begins with Date 21090 representing the date of occurrence of key events. Provider 21100 represents an associated provider for such an event. Location 21110 represents the geographical location of the event. Procedure 21120 represents the occurrence of a medical procedure. HbA1c 21130 represents results from a medical test. X-Ray 21140 represents a diagnostic test. Within the X-Ray data field, an icon 21080 exists by which a user can launch a different representation of the presented data. Meds 21150 represents patient medications. Lab Test 21160 represents a laboratory test. In this example, the Lab Test data field is highlighted, for example in color, to denote an alert based on key data within the column, or logical grouping of information. Within the Lab Test field, an icon 21085 exists by which a user is enabled to launch a different interface, in this example, an Order Interface. Biopsy 21170, highlighted, for example in gray, represents a Flowsheet Header Row from a different Persona, in this example, Specialty 3, also highlighted, as this is important information which may not be normally viewable under the Primary Care Persona. Plan 21180 represents a provider's Assessment and Plan of Treatment for a patient. Financials 21190 represents the financial circumstances regarding the patient, patient's insurance, and other relevant financial information.

In FIG. 25, the Flowsheet 21200-21360 is an array of patient information graphically represented in accordance with the Date 21090 column. At 21200, a Future Appointment is depicted, scheduled for a date, but not yet with a specific Provider. Such future appointments inform a provider as to future plans for either the provider, or any persona, to see the patient again. At 21210, we see a Future Order. Said order is scheduled for a Date, with a Provider, for an X-Ray 21220. At 21240, a Next Visit is depicted. In this example, the Next Visit is not scheduled for a Date, Provider, or Location. What has been specified for the Next Visit is a Lab Test 21250, which can occur at any Next Visit. Each of these three events, Future Appointment, Future Order, and Next Visit are all exemplified as Truncated Data Representations 20030 of FIG. 14. The Future Order of an X-Ray 21220 and Next Visit Lab Test 21250 are Linked Image Data Representations 20070 of FIG. 14, yet without the relevant data to link to, as they have not yet occurred. At 21230, a Scroll Bar is presented, denoting that there may be more information below the viewable portion, and provides access to the information.

Today's Visit 21270 is specific to an event occurring on the present Date and lists a Provider and Location. Also denoted for Today's Visit is a Procedure, a Shave Biopsy, which has occurred with Specialty 3 and has been added to the Flowsheet at 21290. An HbA1c result 21280 is an Alerted Data Representation 20040 of FIG. 14, as well as an Informational Data Representation 20050 of FIG. 14. As such, the field is Alerted by highlighting in red, and offers a colored (e.g. green) corner notification which can be interacted with to see more information. The Financial information 21260 is a Symbol Data Representation 20110 of FIG. 14. In this example, symbols may represent payment status of different categories, such as Office Visits, Procedures, and Diagnostic Tests, General Financial Status for the patient, Percentage of Deductible Paid, Likelihood of Insurance Denial, and Patient Responsible Balance. Symbols are configurable, and as such can be used to represent any data which may be relevant to a logical grouping of data represented.

Past Visits 21300 are shown below Today's Visit 21270. In this Past Visit, a Medication 21310 is denoted by a Graphical Data Representation 20100 of FIG. 14. In the embodiment of FIG. 25, a colored bar (e.g., magenta bar) exists denoting the start and stop dates of the medication, between a first color represented (e.g. teal) medication and a second color represented (e.g., yellow) medication. Graphical representations of medications can conform to existing color specifications or custom configurations. The Plan 21320 is denoted by a Text Link Data Representation 20090 of FIG. 14. As such, interacting with the data will provide more information, such as the ability to read the full Plan.

At 21330, a Thumbnail Image Representation 20080 of FIG. 14 is depicted. A smaller view of an image is displayed to convey a quick overview of the underlying image. 21340 is a Linked Image Data Representation 20070 of FIG. 14. This provides enough information to inform the user that there is an underlying image, which can be directly accessed through interaction with the data representation.

At 21360, a Canceled Visit is depicted. Such a visit is shown as a Missing Data Representation 20060 of FIG. 14, to depict that there was an expectation of data, but it was not present. There was enough information to show a date, provider, and location, but because the patient did not arrive, nothing further is available. An Informational Data Representation 20050 of FIG. 14 shows in the Location logical grouping, which can convey whether the appointment was canceled by the practice or patient, missed, or rescheduled.

A Flowsheet Summary Row 21370 at the bottom of the Flowsheet is comprised of Summary Data Representations 20140 of FIG. 14, and can contain values such as how many visits occurred with each provider, at each location, how many of each type of procedure occurred, highest/lowest HbA1c results, total count of diagnostic tests, a summary of insurance patient balances, and the like. As a Summary Data Representation, each representation can be implemented to interact with data representations within the logical grouping of data associated with the field, such as filtering or placing an order.

A separate module 21380 exists in the bottom right, in the embodiment of FIG. 25 to display Problems, Medications, and Allergies, 21400, a common subset of medical data. Problems is the selected tab, yet Medications is highlighted, for example in color, and as such, a representation of medications 21430 exists within the Problems tab, utilizing Linked Data Representation 20180 of FIG. 14. Such a module is a Linked Data Representation, as is the Module at 21390. Assessment and Plan 21410 denotes a main function of the module of FIG. 25, yet it can be interacted with to change the function of the module, or rules can automatically determine the most important information to be represented. A Date Selector 21420 enables the context of this module to change to a selected date or can represent a date the user interacts with on the flowsheet. This exact information from the Assessment and Plan for the selected date is shown at 21440.

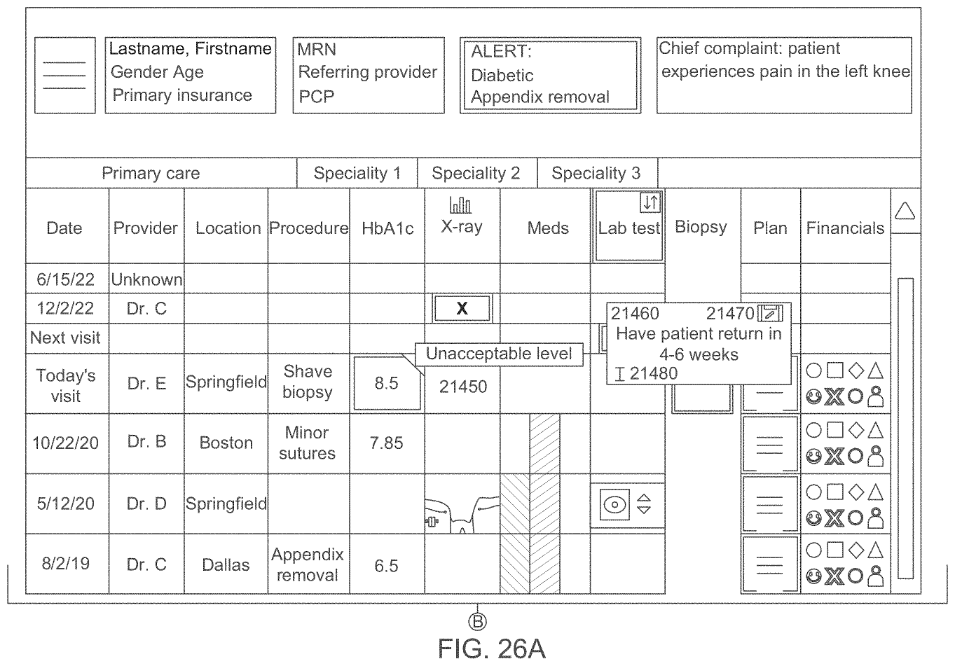

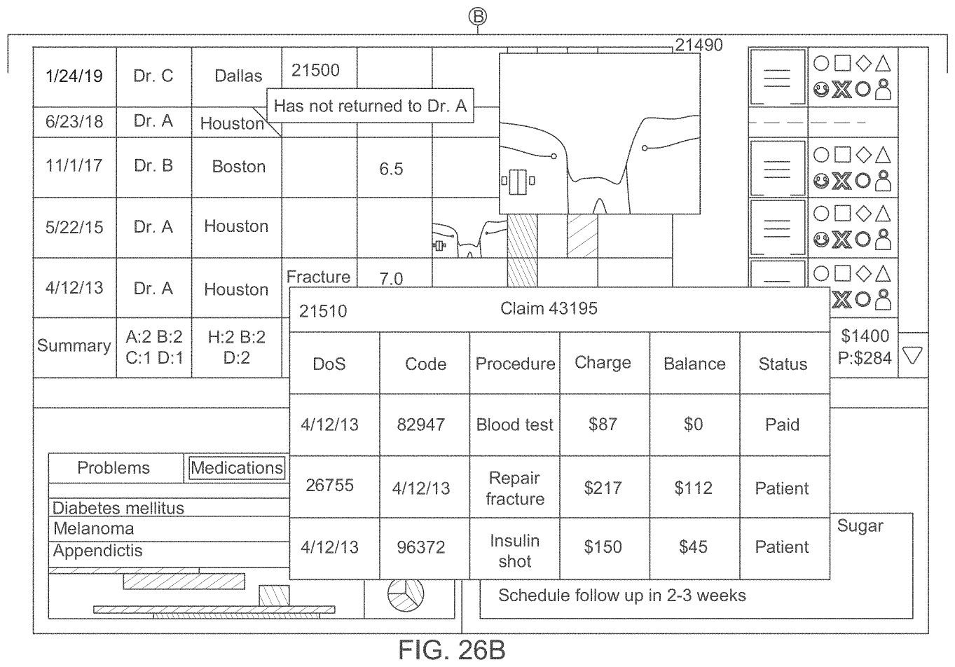

Several Dynamic Data Representations enable access to further information. For example, FIGS. 26A and 26B, (Hereinafter FIG. 26) depict a Dashboard display of Dynamic Data enabling access to additional data in accordance with an embodiment of the present principles. The HbA1C Informational Data Representation at 21450 can be shown upon interaction using a hover, select, or other operation to select the relevant data. At 21450 of FIG. 26, it is depicted that the HbA1c is at an unacceptable level. The Plan Text Link Data Representation interacted with at 21460 displays the full Assessment and Plan. In FIG. 26, an icon at 21470 denotes the field is editable. As such, a text cursor is displayed at 21480 and the user can edit the plan. An Image Thumbnail Representation interacted with can display the full-sized image as seen at 21490. The Missed Appointment Informational Data Representation interacted with denotes the patient has not returned to see the provider their last appointment was scheduled with at 21500. The Financial information Symbol Data Representation interacted with shows a patient's charge status for the respective visit date at 21510. Each representation provides more information, as well as direct access and the ability to edit information.

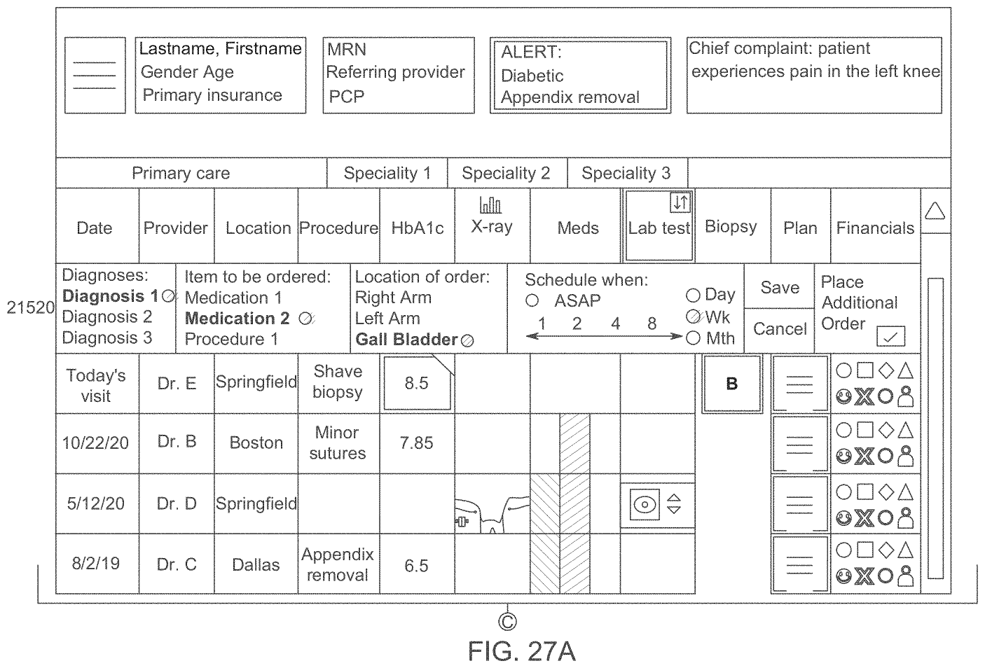

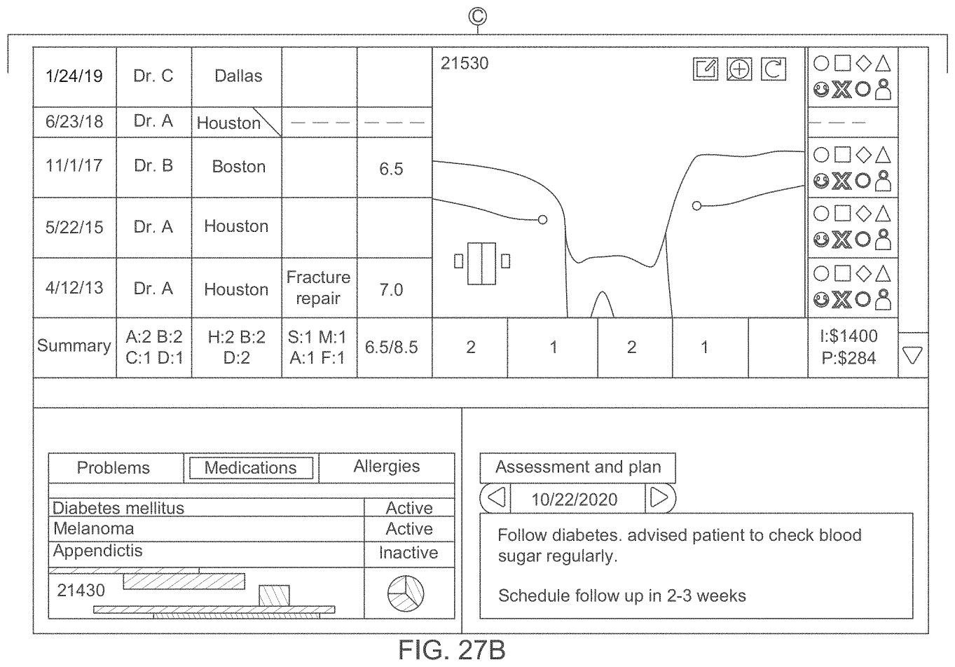

FIGS. 27A and 27B, (Hereinafter FIG. 27) depict an embodiment of a Dynamic Data Flowsheet in which the actual Flowsheet is being altered to allow for additional and/or different sized representations. At 21520, a full ordering panel is depicted as will be described in greater detail below. In the embodiment of FIG. 27, however, future visits have been removed to make room for the ordering panel. Also represented within the ordering panel are colored alerts for Procedure, Medication, and Gall Bladder, to inform the user if there is a discrepancy in their selections. At 21540, a Thumbnail Image Representation is depicted expanded to take up several adjoining data representations to show the full image within the data representations.

FIG. 28 depicts an embodiment of a Dynamic Data Flowsheet including an order in the process of being placed. In the embodiment of FIG. 28 space is made for a new row to be added at 21550, showing that Medication 1 is set to be ordered in accordance with the specified parameters. A colored notification exists at 21570 to inform the provider that the patient has an issue related to a Lab Test, such as one being indicated based on prior conditions and risk factors. At 21560, the intended order details are depicted, no longer alerted because the provider has properly selected compatible Procedure, Medication, and Location. The provider can now review the order, ensure that they have selected what they intended, and can confirm the order using the Confirm button. Additional orders can subsequently be placed by leaving the Additional Order check box checked.

In the embodiment of FIG. 28, below the order, several events have now been hidden. Interacting with Summary Data Representation 21580, relevant data can be filtered. A single interaction can only display those rows with relevant values. For demonstrative purposes, a context menu 21590 is displayed to illustrate complex filtering functionality. In this case, Value of Procedures is selected, and as such, only events containing Procedures are now displayed. Several Summary Data Representation filters can be implemented at the same time, and can be configured to work based on user selection, such as instantiating the Order Process, which can automatically hide or show portions of the application relevant to the process.

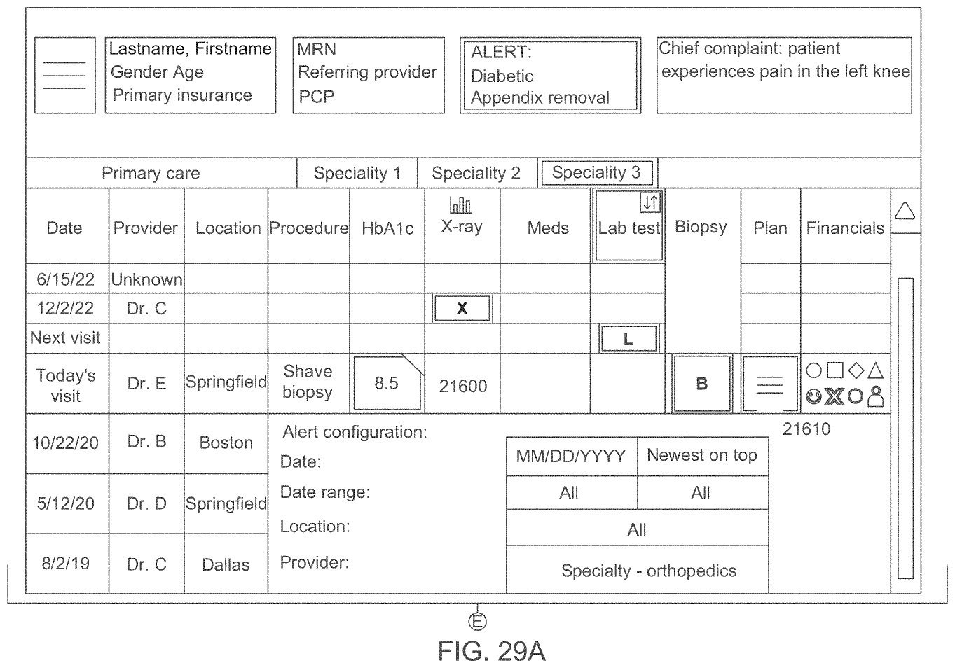

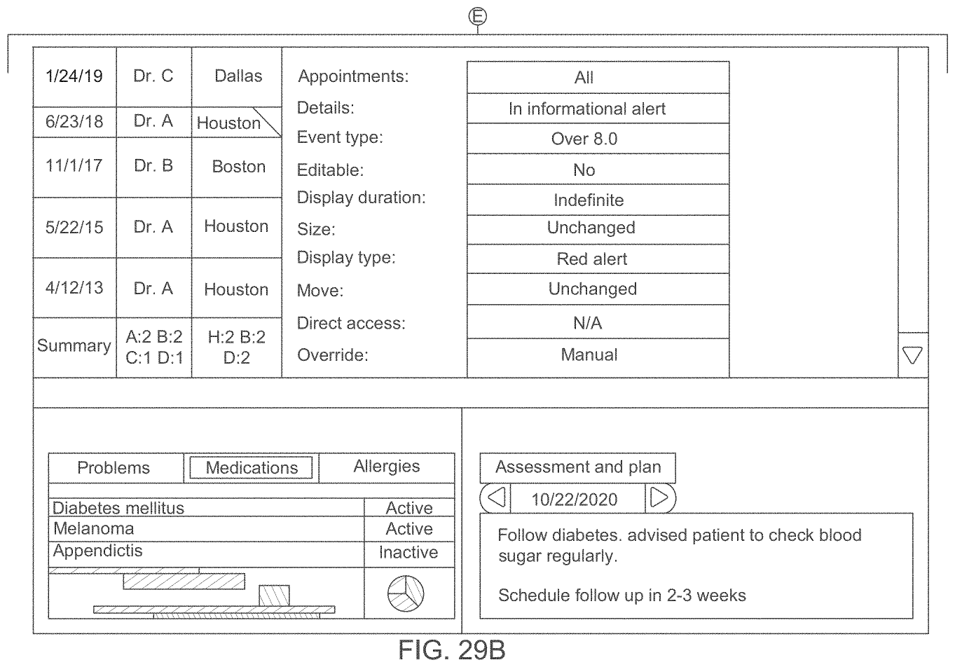

FIGS. 29A and B, hereinafter FIG. 29, depict an embodiment of a Dynamic Data Flowsheet including an Alerted Informational Data representation at 21600. That is, configuration of Dynamic Data Representations can occur within the context of the underlying data so as to ensure proper configuration is being entered. In the embodiment of FIG. 29, an Alerted Informational Data representation is depicted at 21600. In some embodiments, such a representation can be configured by interaction directly with the field in a panel. Instantiating the Alert Configuration module 21610, parameters can be set in accordance with FIG. 15. Specific selections within this example show that for an Event Type of Over 8.0, a colored alert will display, and Details will show in an Informational Data Representation.

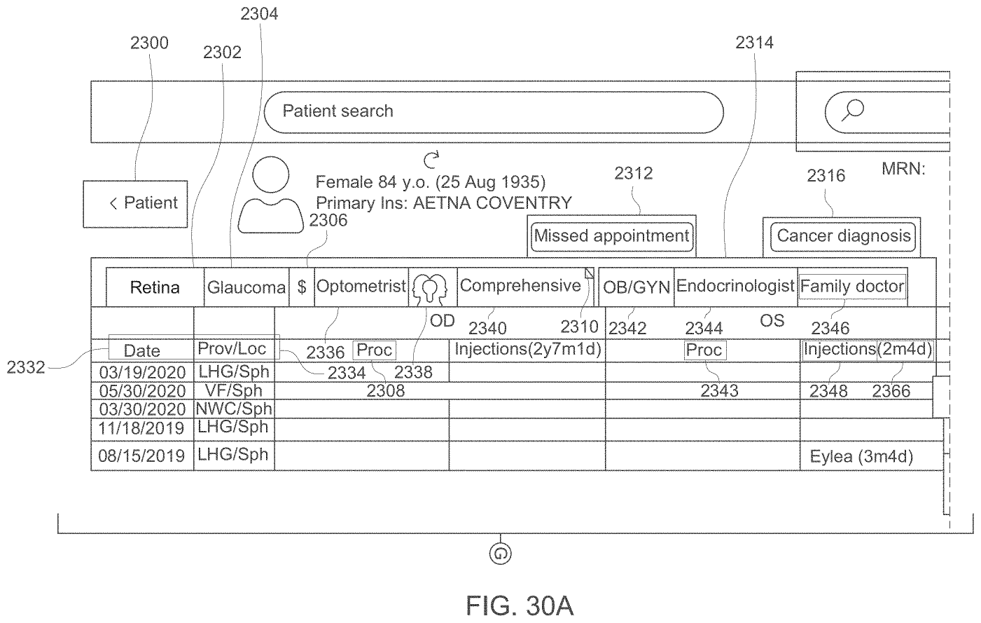

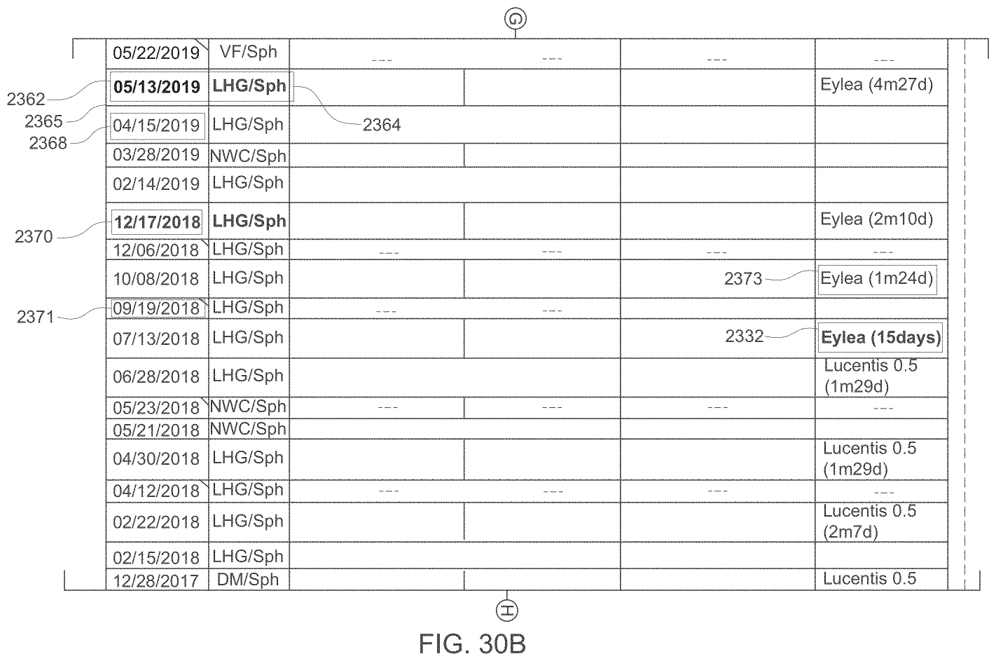

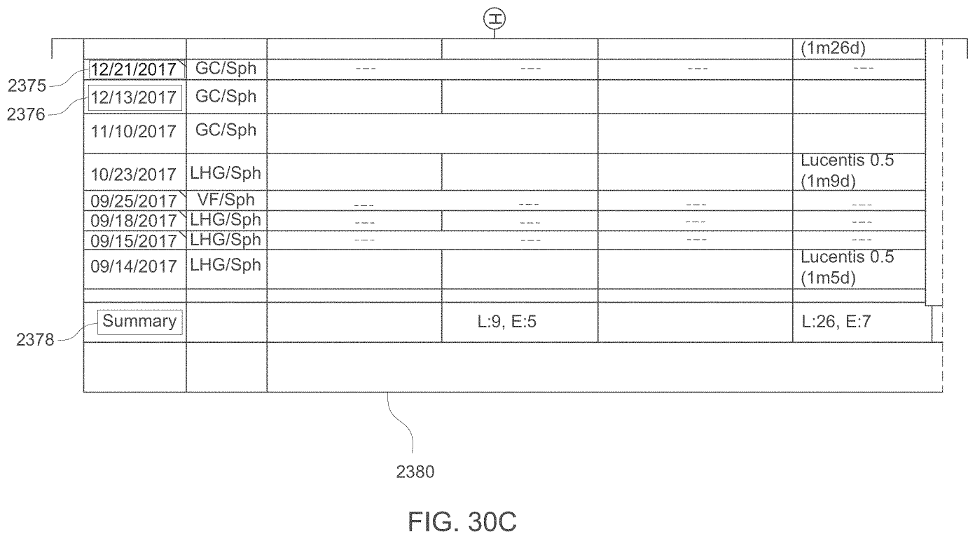

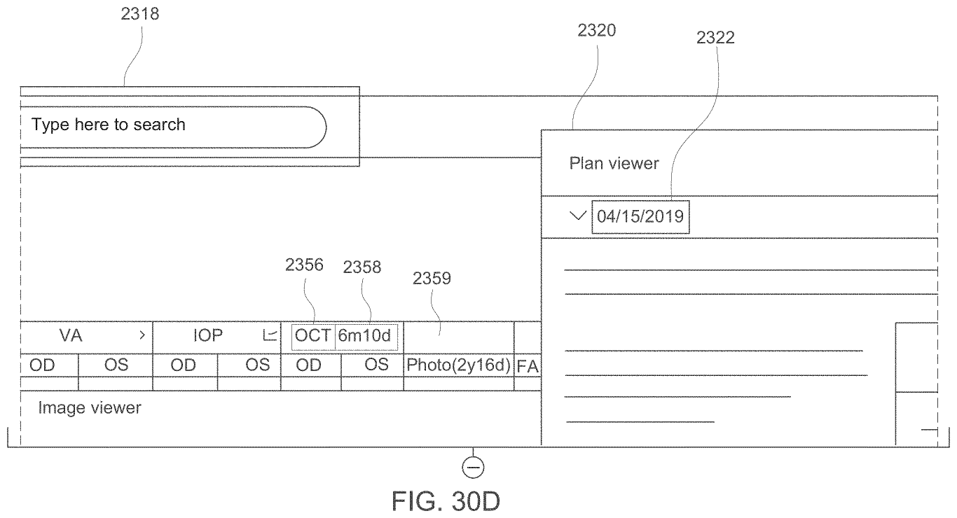

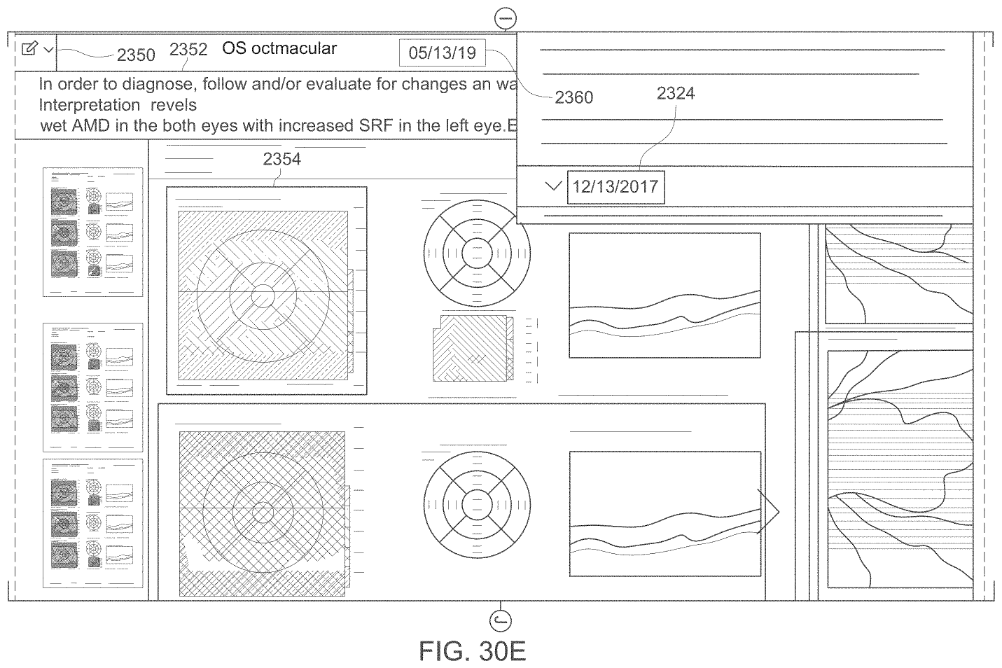

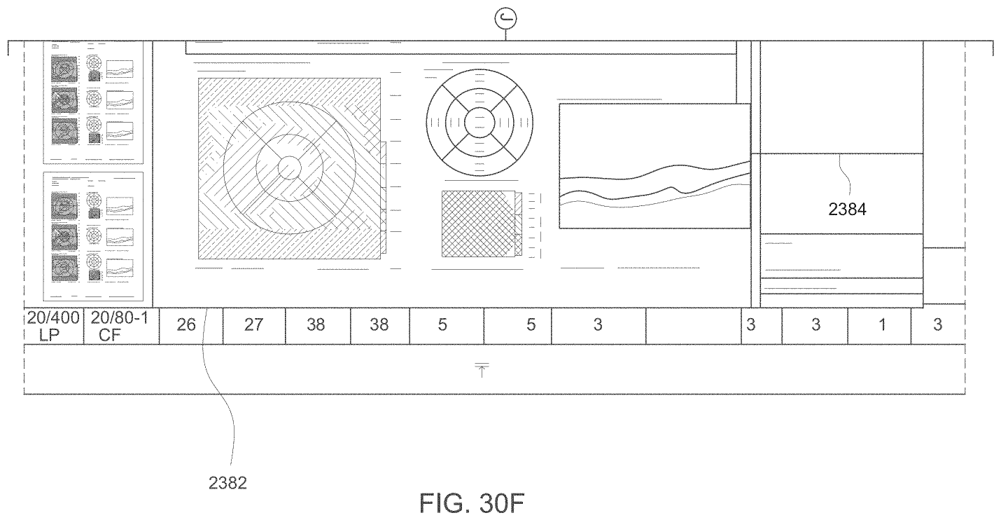

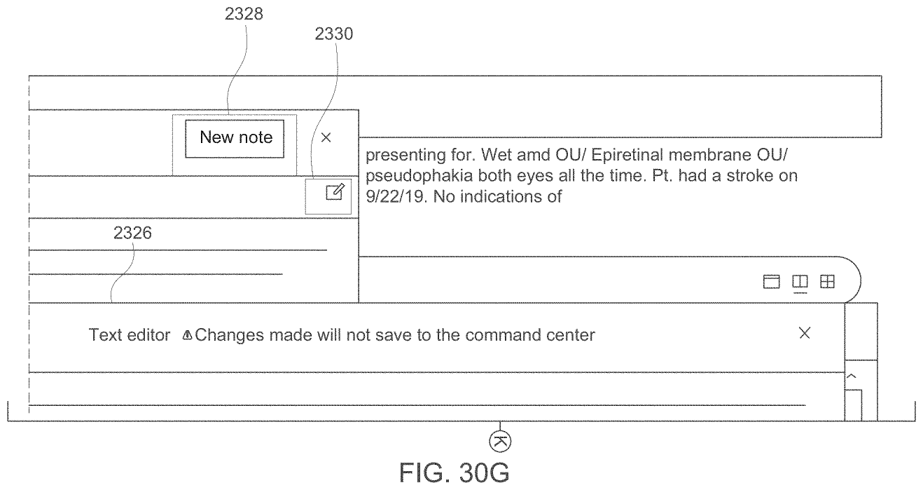

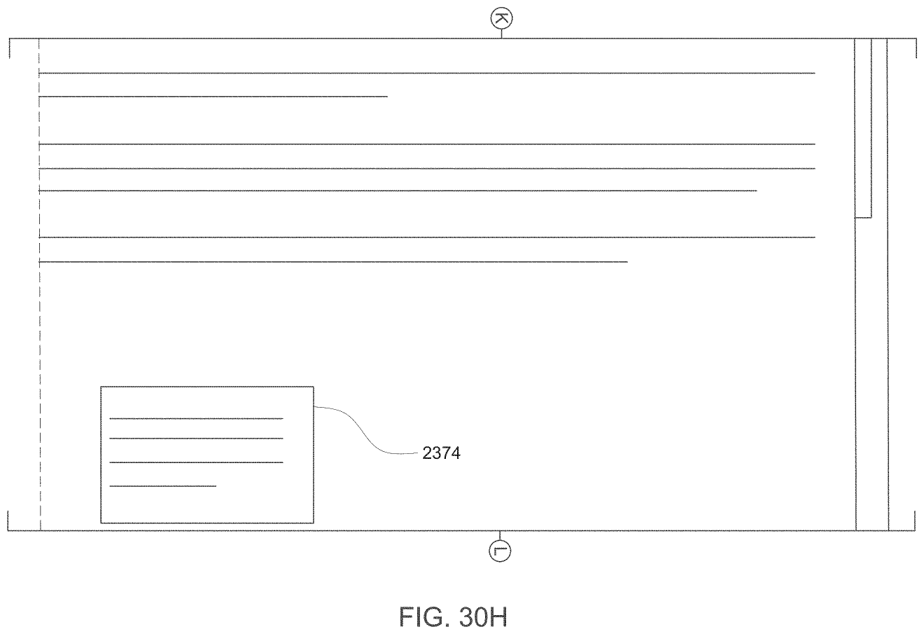

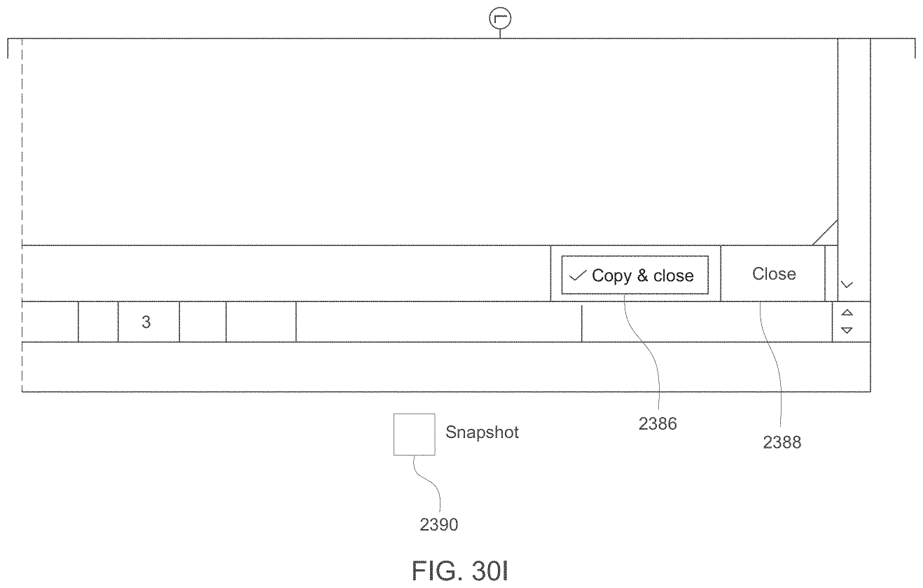

FIGS. 30A-I, (Hereinafter FIG. 30) depict an embodiment of a medical records dashboard in accordance with another embodiment of the present principles. In accordance with the present principles, the medical records dashboard of FIG. 30 is intended to provide and display to a user/medical care provider with all patient data % information necessary to perform accurate and efficient patient care using a single display. In the embodiment of the medical records dashboard of FIG. 30, panels 2380, 2382, 2326, 2320, and 2314 are some examples of different panels that can be moved around, toggled, simultaneously active (i.e., information from each panel can be assessed interchangeably without changing views) and displayed while critical information is viewed. In each column, what is an important data element over time can be followed as noted in column 2332. This enables a user to view the information vital to evaluation of their patients. In addition, in some embodiments, the medical records dashboard of FIG. 30 enables, direct access to patient data/information (no more than one click, one hover or selected directly in any manner). Some embodiments enable toggling by a mechanism such as alt-tab to gain access to underlying patient data/information or associated screen, tab, or window. A user/medical care provider is able to decide what is important to pull up, directly to view, and can move the separate windows or other pop-ups out of the way to view important patient data/information underneath. In one embodiment, a Rules module, such as the Rules module 004 of the Data Command center 001 of FIG. 1, can be configured to know what information for the patient is important, what information must not be blocked, and when information is directly clicked and displayed, enables the movement of a needed columns into a set area on the screen where critical information remains in view. In the embodiment of FIG. 30, an example of two data sets that remain in view is depicted by column 2332, which includes the date of service when an encounter occurred with a patient, and column 2334, which displays the provider and location of encounter. In the embodiment of the medical records dashboard of FIG. 30, all of the other columns, such as column 2348, which depicts injections performed on a patient and/or procedures column 2308 can be moved or at least partially covered from display.

Alternatively, or in addition, in some embodiments none of the patient data/information is completely blocked from view through the use of transparency viewing. In FIG. 30, block 2354 displays an image of an OCT that displays to a user/medical care provider if injections of the left eye are working. In the embodiment of the medical records dashboard of FIG. 30, column 2348 is viewed, not blocked, so the user can correlate when the injection (or any procedure of clinical information or diagnostic test) was performed and how it relates to the information that was pulled up, with direct access to any additional information. In some embodiments of a medical records dashboard of the present principles, columns/windows/pop-ups of interest to a user can be moved to another portion of the medical records dashboard where no patient data/information or patient data/information of little or no interest to a user, exists. For example, if the user would also like to compare OCT data 2356 and in particular the left eye, as this example shows injections of certain medications (i.e. Eylea, Lucentis) and column 2348 over time, the user could simply drag 2356 or just 2358 (left eye) over to column 2308, because no data is present in that area of the medical records dashboard. Now all in one view and in a particular section of the medical records dashboard, exists all information that user would need to compare OCTs 2356 over time with injections 2348. In another example, when a photo 2359 is being compared to when an injection is done in the left eye 2348, then 2359, can be moved, dragged, or automatically be placed in location for example next to or in place of 2343. A user remains in control and able to move items out of view and by activating icon 2390 can take a snapshot (record) of a current arrangement of the medical records dashboard such that a record of the arrangement can be stored.

Simultaneously, a medical records dashboard of the present principles enables a user/medical care provider to recall and view plans of the past by activating a plan or A&P column or a particular plan in a column. The medical records dashboard of FIG. 30 enables current and past plans to be simultaneously displayed. As such, in context, a new note could be created in block 2328. A medical records dashboard of the present principles, such as the medical records dashboard of FIG. 30, enables images, procedures, dates of service, plan, or any other patient-related data/information, such as clinical measurement, i.e. VA (vision OD 3005—right or OS 3006—left), to be compared in context. By way of example, how a treatment is working as measured by an image, clinical parameter, or any other related data set can be interpreted and noted in the medical records dashboard in at least block 2352, which can be a new interpretation and can be edited by activating icon 2350. In one embodiment a plan viewer can be accessed by activating block 2328 and a new note or the editing of an old exiting note 2330 can be accomplished via a text editor window 2326. In the embodiment of the medical records dashboard of FIG. 30, a user/medical record provider is enabled to type or dictate a note 2374 accurately while relevant information is viewed in for example a window. Although in the embodiment of FIG. 30 the medical records dashboard only provides a user/medical care provider one means for editing notes, in some embodiments, a medical records dashboard of the present principles can provide a user/medical care provider many ways to edit notes.

In the medical records dashboard of FIG. 30, panel 2314 enables a user/medical care provider to select to view patient-related data/information from a number of different health care providers, such that patient-related data/information from every medical care provider that has ever cared for a patient can be viewed by, for example, all other specialties who provide care for that patient. For example, in FIG. 30, a user/medical care provider can select to see patient care data/information related to a retina specialist 2302 and/or a glaucoma specialist 2304. In some embodiments, sharing of patient-related data/information from other users/medical care providers can require permission from at least one of the patient and the other user/medical care provider.

In the medical records dashboard of FIG. 30, the panel, arranges patient data/information displayed in rows and columns. Users/medical care providers can have dashboards that are similar in display because the users/medical care providers charge, order, or perform similar CPT codes and often treat similar ICD diagnostic codes. Type of eye doctors are listed in order in this example # 2302 (retina), 2304 (glaucoma), 2336 (optometrist), and 2340 comprehensive eye doctor.

In the embodiment of the medical records dashboard of FIG. 30, the different users/medical care providers can let all the other providers know something is important by highlighting the tab 2302, 2304, 2342, 2344, and 2346 in the medical records dashboard view of other users/medical care providers. In such embodiments, a user/medical care provider is able to hover or otherwise active the highlighted tab to bring into view a message 2312 that can detail an important aspect of patient care for the corresponding other user/medical care provider. As depicted in FIG. 30, a current user/medical care provider is alerted that a patient has missed appointments with a corresponding user/medical care provider. In another example, a tab to a family doctor 2346 could light up or blink or in any way get a user's attention to indicate that an event is particularly important. In another example and as depicted in FIG. 30, when activated by a user/medical care provider, over a blinking endocrinologists tab 2344 can appear an alert window 2316 that can inform a user/medical care provider that a patient has received a diagnosis of cancer. In some embodiments, such important messages can be caused to display without requiring a user to activate or hover over a blinking or colored specialist tab.

There are situations where doctors, even if in separate practices and separate specialties, what they do can impact what another doctor does. By way of example, a retina surgeon injects many times in an eye, up to 12 times a year. But, clearly, if a family doctor discovers cancer that might change the frequency a retina doctor may want to inject. If a patient has a stroke, there are some research studies that suggest the medication that one doctor is using, in this case displayed 2348 injections in the eye, by a retina surgeon might increase the risk of another stroke. In some embodiments, a Rules module of the present principles, such as the Rules module 004 of the Data Command center 001 of FIG. 1, is configured to recognize such situations in which treatment by one doctor can affect a treatment by another doctor and, in such instances, the Rules module 004 is configured to generate an alert to be displayed to all users/medical care providers of such situations.

There are many different ways that embodiments of a medical records dashboard of the present principles can display important information. By way of another example, at any time, if an important event occurs in any encounter of any provider, the information can be inserted into a row in chronological order, where it makes sense, to show on a timeline that the event occurred. So, if it was discovered that the patient had a stroke on May 25, 2019, as depicted by number 2362 in FIG. 30 the initials of a caring provider can be displayed under the provider instead of a current provider as depicted in FIG. 30 by 2362 marked as 2364. The difference between providers can be highlighted in many different ways. If it's a provider that is not normally on a row on clinical panel 2380 or for example in this case, illustrated as an example of a retina doctor provider, then this new provider with a row can be highlighted or be a smaller row or a larger row. Also, instead of having the normal information in columns, because the other provider might not perform similar CPTs, instead in some embodiments there can be displayed, at the end of the row in a specially designated area for outside attachments or notes, information and it can be identified if the information is from a different provider.

In the embodiment of the medical records dashboard of FIG. 30, 2306 can include financial data, and in this example shows ‘$’ sign. In such embodiments, access to financial data can be limited to only user/medical care providers credentialed to have access for instance only the users/medical care providers and colleagues in their practice can have access. In the embodiment of FIG. 30, icon 2338 can be activated to enable access to financial data to different users/medical care providers. For example, in FIG. 30 2336 is an example of an optometrist and 2338 depicts an icon with appearance of two faces which can represent sharing access.

In the embodiment of the medical records dashboard of FIG. 30, the glaucoma specialists 2304 has entry 2306 next to it, which can be used to launch a revenue cycle management (RCM), which is just one mechanism that any user/medical care provider can use to get more information in regard to their own practice's billing or any other information. By way of example, in the embodiment of FIG. 30, activating icon 2306 can enable access to a user/medical care provider to cost, charges, any financial information payments, rejections, to which the user/medical care provider has access. In one embodiment, the financial information can comprise a mirror-image of the clinical dashboard, so a doctor, by toggling back and forth, a transparency or overlay can be used to determine what was charged, paid, rejected, or authorized for every service performed. Alternatively, or in addition, clicking on RCM 2306 on the same view or on the same scanning screen the information that is financial in nature can be displayed under, over, above, or superimposed, similar to transparent paper, with one embodiment, the billing function, being behind or lighter and clinical being darker or vice versa. In some embodiments, each row of panel 2314 can have 2306 or 2338 next to every one of the tabs (actionable dashboards of different providers).

In some embodiments of the present principles, a user of a medical records dashboard is identified upon use. For example, in some embodiments, a user/medical care provider is required to provide identifying information when the user/medical care provider wants to use a medical records dashboard of the present principles. In some embodiments, a user/medical care provider can provide predetermined configuration information to identify how a medical records dashboard should be displayed for that particular user. For example, in some embodiments a Rules module, such as the Rules module 004 of the Data Command center 001 of FIG. 1 can have access to configuration information for a medical records dashboard provided by a user. In such embodiments, the Rules module 004 can be configured to arrange and cause a display of the medical records dashboard in accordance with the predetermined configuration information provided by the user, for example, upon initiation of the medical records dashboard by the user.

Alternatively, or in addition, in some embodiments, a user/medical care provider can drag and drop portions of a medical records dashboard to arrange the medical records dashboard into an arrangement that is best for the user and/or the user's practice or in some embodiments, into an arrangement that is best for a particular patient. For example, an eye doctors might care more about a condition like diabetes, so any doctor that takes care of diabetes, endocrinologists, family doctors, kidney specialists, urologists tend to have more patients and procedures related to diabetes than other specialists, like a radiologist.

In the embodiment of the medical records dashboard of FIG. 30, when a user selects entry 2330, window 2374 is displayed for inserting notes, which can then be saved and closed by selecting 2386, or just closed by selecting entry 2388.

Tab 2348 of FIG. 30 is a tab for providing a user information regarding injections given to a patient, and tab 2366 of FIG. 30 can provide quick information about the injections including a number of injection or a type of the injections. In FIG. 30, 2372 depicts the identification of an example of an Eylea injection having been performed on Jul. 13, 2018, and it is red but can be highlighted in many different ways. In 2372 adjacent to Eylea it says 15 days which in this example count from the last time an injection in the eye was done. In the embodiment of FIG. 30, the medical records dashboard depicts that Lucentis was injected Jun. 28, 2018 which is only days earlier from a Jul. 13, 2018 injection of Eylea and the column counts in the embodiment from one to the other. In some instances, procedures of Eylea or Lucentis injections are allowed only every 28 days from each other. In embodiments of the present principles, a Rules module, such as the Rules module 004 of the Data Command center 001 of FIG. 1, can be configured to have access to information, including but not limited to, rules regarding how frequent or far apart medications can be given, and in some embodiments, the Rules module 004 is configured to cause the display of an alert if a user/medical care provider is attempting to order a procedure improperly or if procedures have already been performed improperly.

In the embodiment of FIG. 30, the panel can be used to display diagnostic test and images. In the embodiment of FIG. 30, when tab 2350 is selected an interpretation panel 2352 is opened, which can display notes of an interpretation of patient care that could be actually written on the day of treatment. Element 3254 of FIG. 30 is an image of a test performed on the patient.

In some embodiments, image icons, representative of results of test performed on a patient, can be selected to cause a display of an underlying corresponding image, such that a user/medical care provider can, in context, make a determination of the test and see the actual test while knowing whether there was a procedure or in this example a medication injection done, as depicted in 2366.

The embodiment of the medical records dashboard of the present principles of FIG. 30 illustratively includes a search box 2318. The search box of the medical records dashboard of FIG. 30 can be used to search for a doctor, a date, an image, particular procedures, a particular diagnosis, payment rejections and payments and substantially any other patient related data/information related to the medical records dashboard. In some embodiment, the medical records dashboard can instantly reconfigure based on what is searched and can be configured to display only the portions of the medical records dashboard for which search results are returned. Combinations of queries can be searched. For instance, show only the rows and dates of service with the diagnosis of diabetes that had injections of a particular medication, column 2348. Instantly, only the rows with injections with the patient having a diagnosis of a certain ICD like diabetes or if comparing a particular diagnostic test with a procedure and trying to correlate it, along with a clinical finding, the user could search “show me only the rows and dates of service where the vision was between 20/20 and 20/80” or “the pressure of 16 to 20 that also had the same date of service, a procedure in 2348 of Eylea and also had an OCT. The patient data/information associated with the medical records dashboard can then be searched and in some embodiments, only rows and columns of the medical records dashboard related to the search can be searched.

In some embodiments, a Data Command Center of the present principles is enabled to provide a Customizable, Correlative Graph (CCG). That is, the Data Command Center is able to collate data and visualize correlation between different, related datapoints, each with their own distinct visualizations. Novel to customizable visualizations is to display an array of customized visualizations correlated on a comparative axis or axes. This customized, correlative display consists of one or more visualizations of Command Center data, horizontally, vertically, on a Z axis, or on multiple axes displaying multiple events, results, and/or calculations. In some embodiments, the Customizable, Correlative Line Graph display can be launched from within a Patient Flowsheet using a button, keystroke, or series of keystrokes such as the icon shown in 21080 of FIG. 25. Upon launch, the Customizable, Correlative Line Graph generates a view and can display as a popup, popover, pop out, or otherwise in relation to, but not limited by the launch point. The Graph can overlay or adjoin the underlying Flowsheet in opaque or transparent states, be pinned to the Flowsheet, and/or may hover over or aside said Flowsheet.

Upon initiating the Customizable, Correlative Graph, a series of actions are performed to determine data and format of data displayed. Preconfigured CCG displays can be stored in tables or generated at runtime based on key considerations such as those laid out in Dynamic Data Representations described above.

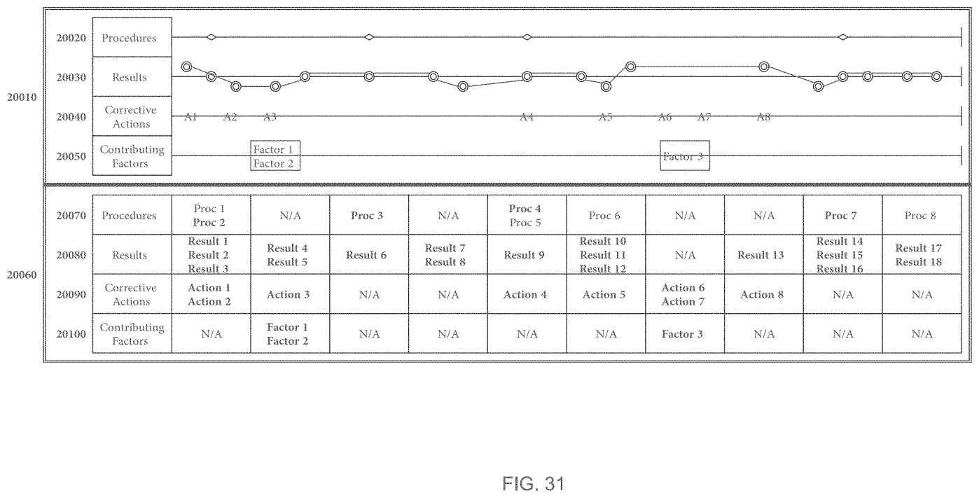

FIG. 31 illustrates a horizontal correlative graph in accordance with an embodiment of the present principles. In the embodiment of FIG. 31, available data (20060) is visualized graphically (20010), as a series of events (20070) graphed against a timeline (20020), correlated with a series of results (20080, 20030), a series of actions (20090, 20040), and a series of contributing factors (20100, 20050). Any number of relevant details and categories of data can be correlated as needed.

Rendered Customizable, Correlative Graphs can be interacted with in such ways as to turn on or off represented values in a similar manner to expanding/collapsing/filtering of Dynamic Data Representations, i.e. turning on or off subsections of data, individual visualizations categorized by logical grouping, selecting only specific elements to display. Selecting data representations within the display, and/or moving elements between positions to achieve a different view, can also be affected based on principals described in FIG. 13 and throughout the teachings herein. It should be noted that additional visualizations can be added, additional flags derived, and a series of rules explained throughout the teachings herein to manifest in the final rendering.

It should be noted that the single axis representation of the CSG of the present principles described above and represented in the Figures does not preclude multi-dimensional representations with multiple parallel representations as well as multiple perpendicular, or otherwise non-parallel representations.

The Customizable, Correlative Graph of the present principles reaches its logical end at which point all data is rendered, processing of rendered data has occurred, and any/all necessary actions have been taken based on the processed data, including, but not limited to, Flags, Alerts, Clinical Decision Support, and Auto-Tasks. Auto-updates to patient data can initiate refactoring of the Customizable, Correlative Graph.

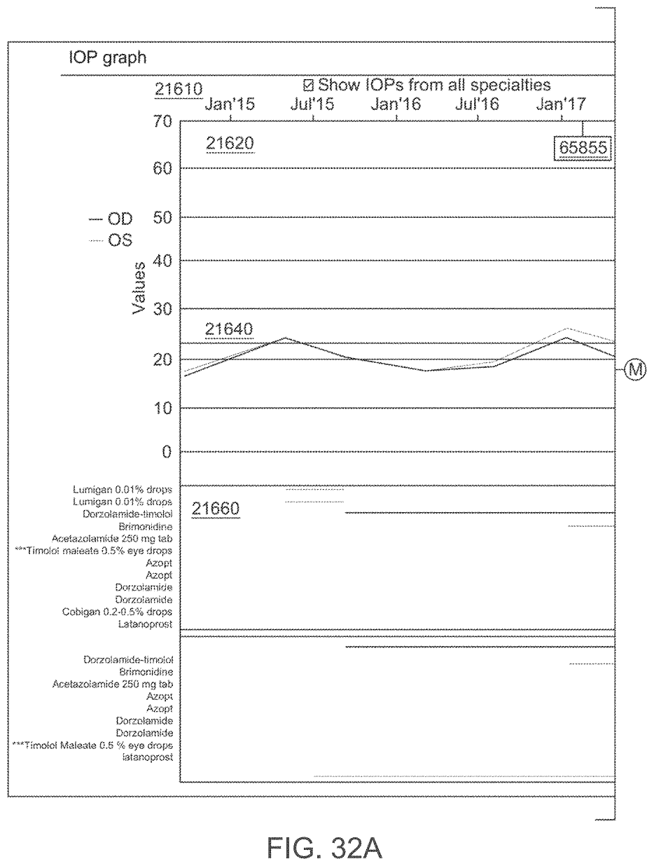

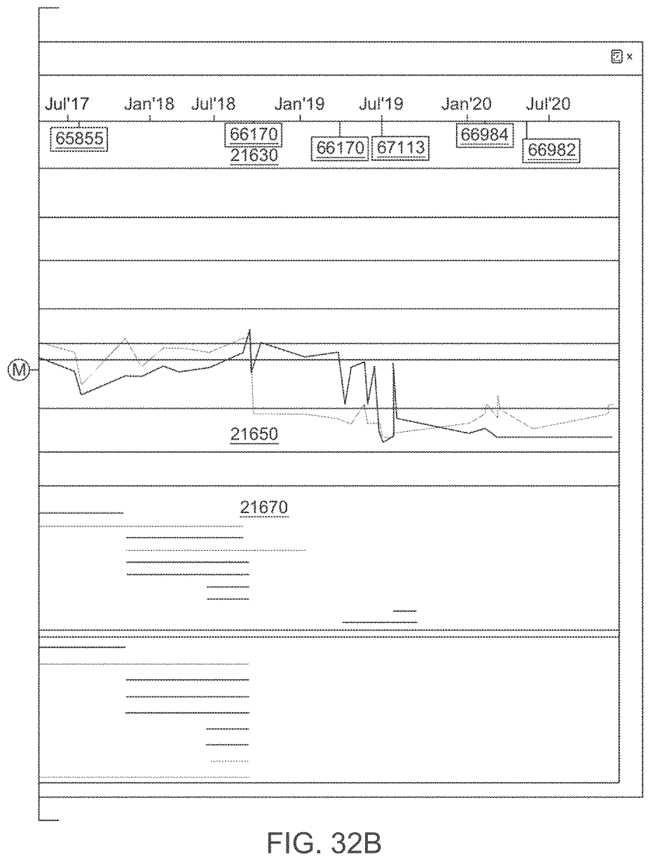

FIGS. 32A and B, (Hereinafter FIG. 32) depict a diagram of a correlative graphical representation of patient data able to be displayed by a Data Command Center in accordance with an embodiment of the present principles. That is, a Data Command Center of the present principles can collate, evaluate, and correlate multiple data sets to represent them in a correlative graph, such as illustrated in FIG. 32. In this embodiment, the correlative graph 21610 is comprised of three sections. The first section 21620 represents procedures by a code such as seen at 21630. The second section 21640 represents pressure, exemplified as a line graph which tracks with the pressure. A large drop in pressure is illustrated at 21650. The third section 21660 represents medications as graphical lines corresponding to each medication. A clear stop of multiple medications is shown at 21670. In the embodiment of FIG. 32, these three factors, procedures, pressure, and medications, are depicted as inherently tied together in treatment.

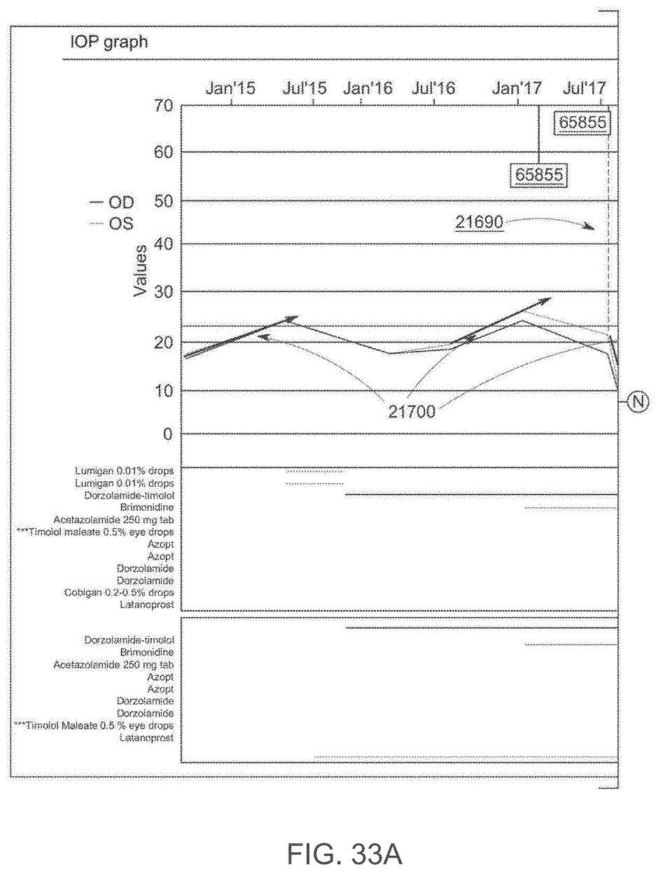

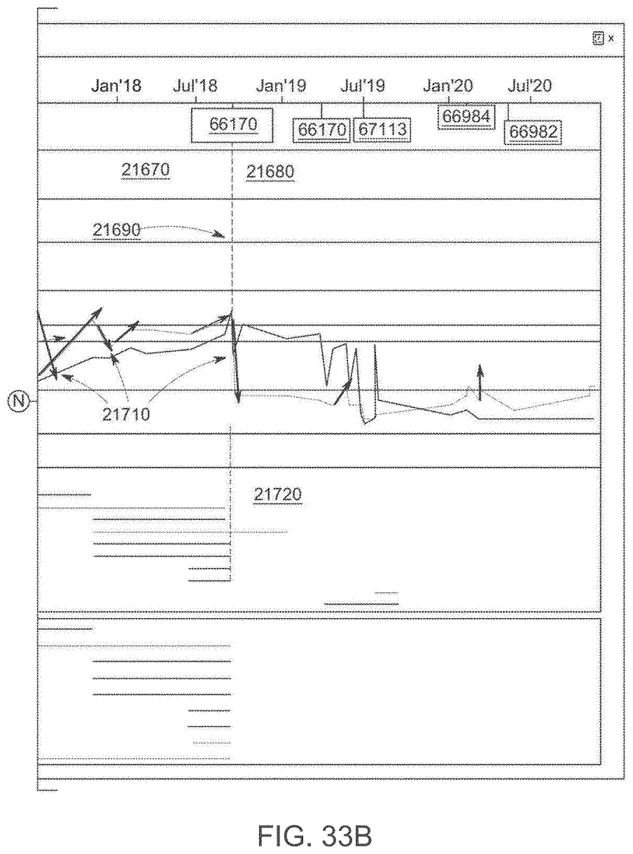

FIGS. 33A and 33B, (Hereinafter FIG. 33) depict correlations made for the diagram of the correlative graphical representation of patient data of FIG. 32 in accordance with an embodiment of the present principles. More specifically, in FIG. 32, at 21670, a procedure is depicted, now larger and highlighted, which correlates to a drop in pressure. A second, larger procedure 21680, now highlighted, for example in color, correlates to an even larger drop in pressure. These correlations are illustrated by the lines at 21690. Arrows of increasing size and differing color now represent notable rises and falls in pressure. At 21700, the first 2 arrows comprise a first color (e.g., yellow) and denote a moderate rise in pressure, while the third arrow comprises a second color (e.g., orange) and is thicker, denoting a significant increase. At 21710, colored arrows denote drops in pressure. The third arrow is clearly thicker, denoting a significant drop in pressure. It becomes clear that the procedure at 21690 is the significant factor in this drop. Another correlation is made at this point. At 21720, a connection to the cessation of several medications is depicted. In the past, such information would have existed in separate areas and associations would need to be made outside of the present application. In accordance with the present principles, in the embodiment of FIG. 32 and FIG. 33 every thing is clearly delineated, correlated, and displayed in such a manner as to reduce the amount of information needed to make connections, and as such, informed treatment decisions.

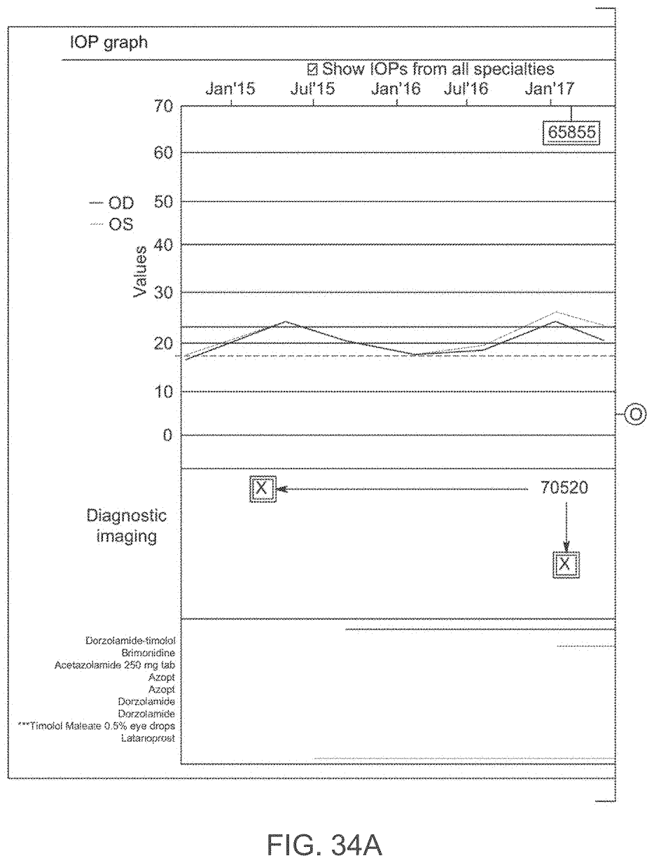

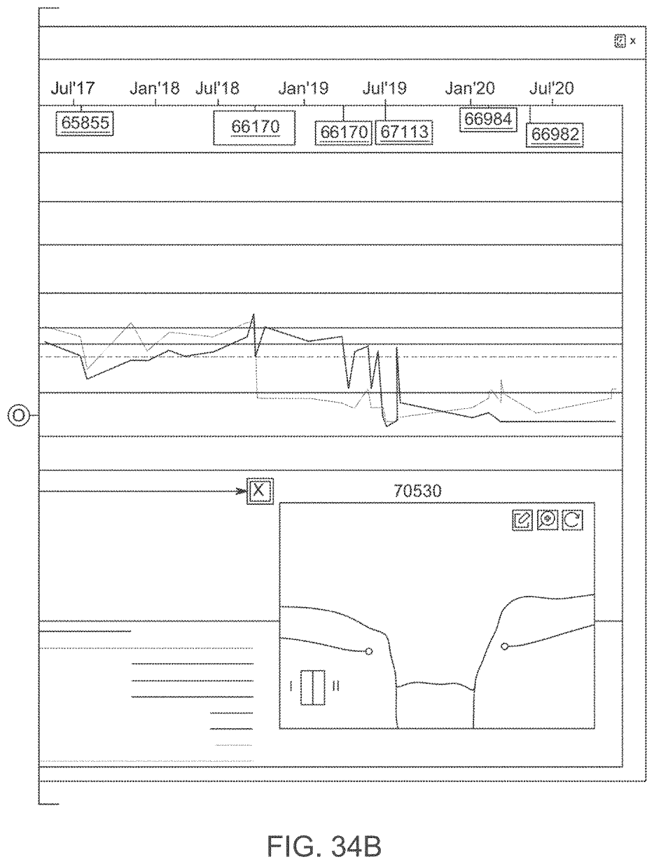

FIGS. 34A and 34B, (Hereinafter FIG. 34) illustrate direct access and parameter setting on a horizontal graph in accordance with an embodiment of the present principles. In the embodiment of FIG. 34, at 70510, a dotted line is displayed denoting a threshold for pressure. Such a line defines the limit at which this patient, or any patient if configured globally, may reach a point at which action is required. Above this line, an event is triggered. This can result in an alert, message, task, or other notification. As the data is interactive, one can drag this line higher or lower to adjust the threshold, updating respective rules to now account for the new threshold set. Illustrated at 70520 are several Diagnostic Images. Each is positioned according to the governing timeline. At 70530, an image is shown being directly accessed from a respective icon, which generates a view of an in-context image viewer. As all data representations can be set to interactive, actions such as generating an image viewer/editor, order panel, bidirectional text editing, or other interface can be accessed.

In some embodiments, if a patient misses an appointment, an auto task can be generated to alert a user/medical care provider schedule that the patient missed the appointment so that another appointment can be scheduled, or to the Clinical trial coordinator if it is part of a research protocol. Even parameters of when to create the task such as two missed appointments in a row can be set. This enables automatic tracking and a user/medical care provider can set it knowing the unique individual issues with a patient and can determine how important a missed appointment might be for a particular patient at a glance by showing previous data projected in the background or through one user interface on another monitor the user/medical care provider can cross check and individualize the alerts and tasks since a single missed appointment may be serious for one patient, but not so serious for another. Even parameters of when to create the task such as two missed appointments in a row can be set.

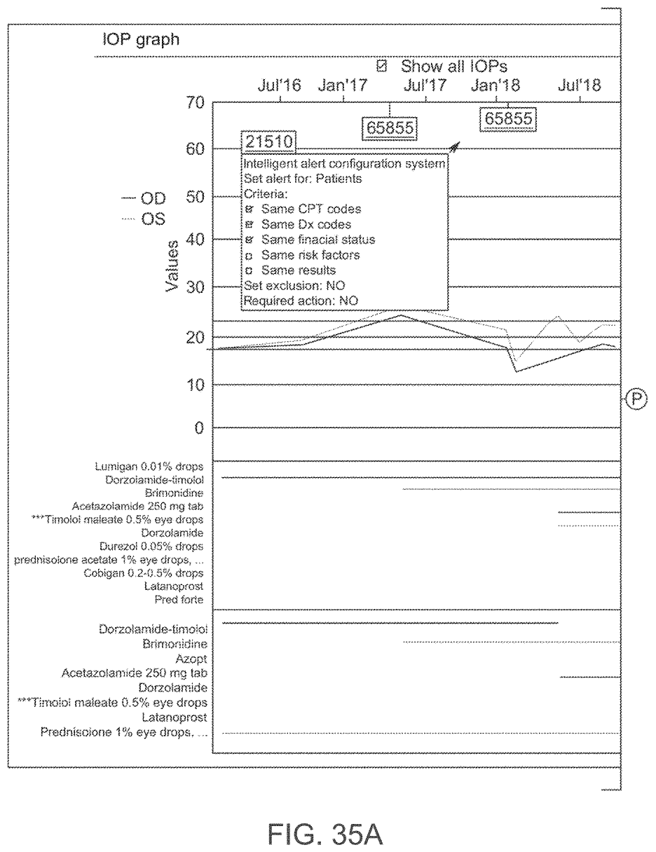

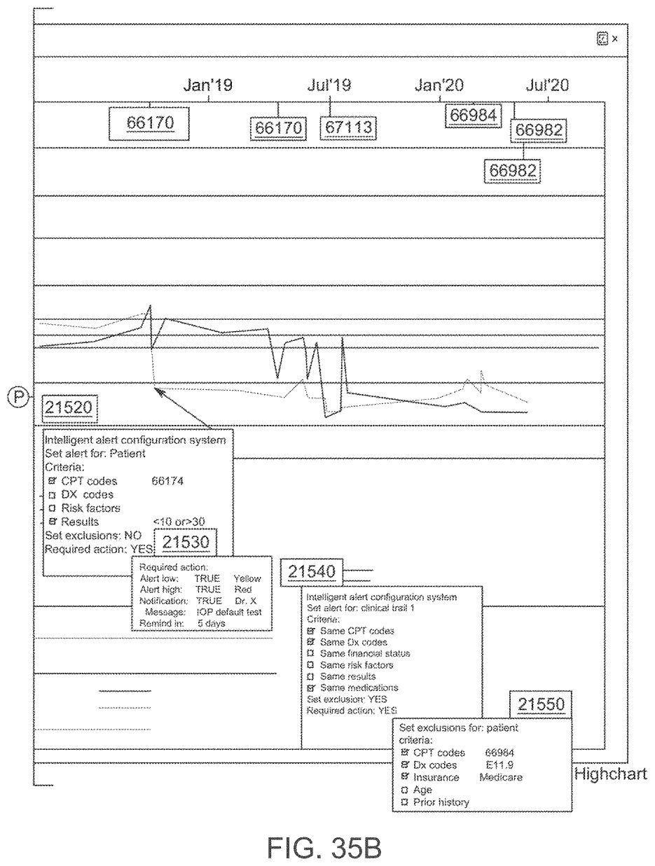

An intelligent alert configuration system, in one embodiment, can be represented in multiple ways, based on several criteria. For example, FIGS. 35A and 35B (hereinafter FIG. 35) depict three different representations of an intelligent alert configuration system overlayed upon several different aspects of an application in accordance with an embodiment of the present principles. The alert configuration system is associated with a panel from which it is invoked, and allows selection of configuration parameters for that panel to modify information displayed in the panel. The alert configuration system of FIG. 35 can, in the case of a procedure 21510, display parameters associated with procedures, patients with like procedures, correlations to diagnoses, financial status, risk factors, results, or other relevant criteria, while enabling for exclusion criteria and actions to be taken, while also being able to be assigned to a patient, group of patients, all patients, or a patient or group of patients with specified criteria. In the case of launching the intelligent alert configuration from a result 21520, a different set of parameters can be specified relevant to that result, patients with similar results, correlations to diagnoses, financial status, risk factors, results, or other relevant criteria, while enabling for exclusion criteria and actions to be taken, while also being able to be assigned to a patient, group of patients, all patients, or a patient or group of patients with specified criteria. An example of required actions 21530 denotes several key actions which can occur upon triggering the alert. These include, but are not limited to, a visual or audio alert, tiers of alerts based on values, notifications to be sent and to whom, reminders are to be sent. In the case of launching the intelligent alert configuration from a medication 21540, a different set of parameters can be specified relevant to medications, patients with similar medications, correlations to diagnoses, financial status, risk factors, results, or other relevant criteria, while allowing for exclusion criteria and actions to be taken, while also being able to be assigned to a patient, group of patients, all patients, or a patient or group of patients with specified criteria. An example of exclusions 21550 denotes several key actions which can exclude a patient or patients from an alert.

In the Whole-Life View of the present principles, all pre-described functionality is aggregated into a single, intelligent view of a patient's whole life. All relevant data underlies the Whole-Life View, but zoom levels add an additional dimension to what is displayed. At its highest zoom level, only the most important factors are displayed. And its lowest zoom level, flowsheet-level access can be achieved. At each zoom interval, reprocessing of rules can occur to include additional data, differing representations of data, and notifications of key events.

Whole-Life View can be accessed from within the context of a Flowsheet, report, or patient list, utilizing a button, keystroke, or series of keystrokes, to initiate the Whole-Life View, such as the icon shown in 21080 of FIG. 25. Whole-Life View can display at a preconfigured zoom level, a preferred zoom level, or can utilize rules to determine the required zoom level based on key factors that can be stored in tables or generated on-the-fly based on key considerations such as those laid out in Dynamic Data Representations, and those laid out in the Customizable, Correlative Line Graph FIG. 31, FIG. 19, and FIG. 20. While within Whole-Life View, zooming can be achieved by selecting a button, keystroke, series of keystrokes, utilizing the mouse, hand gestures, touchscreen, or other logical means of interface. Zooming can also be automated based on rules as defined by the Rules Engine 10180 of FIG. 3, whereby important events can directly affect starting zoom level.

The determination of the importance of data to display in a Whole Life View must account for point-in-time refactoring of data displayed. While a heart attack can be hugely important, overall, if the zoom level is achieved which does not account for when the event occurred, only the events of the specified timeframe will be accounted for in the general view. Critical patient indicators can be implemented to account for events outside of the viewable display. This does not mean that events outside of the viewable timeframe are not of importance and can still affect the display of events in the view, such as the heart attack increasing the importance of a stent procedure within the view. In some embodiments, a weighting system can be implemented to make such determinations.

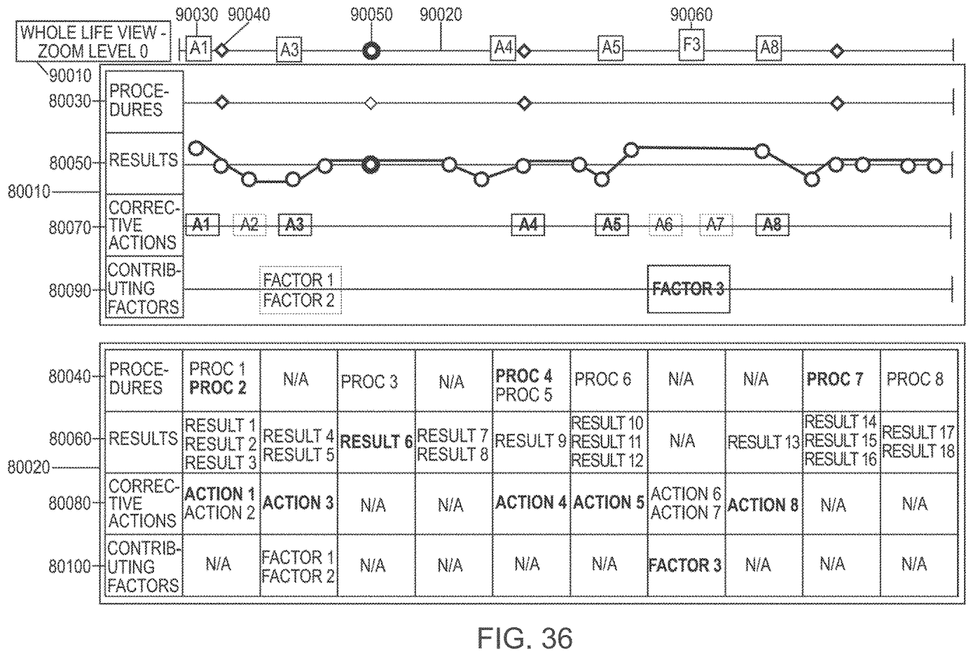

FIG. 36 illustrates a whole life view zoomed out in accordance with an embodiment of the present principles. In the embodiment of FIG. 36, Zoom Level 0 (90010) is achieved, and the entire timeline of the patient's life is displayed (90020). Underlying data (FIG. 80000-80020) is processed and key datapoints are intelligently determined for display. Configurations then apply to intelligently determine how to represent the datapoints. As an example, these datapoints can include all corrective actions (90030), procedures (90040), results (90050), contributing factors (90060), and any other correlated dataset display deemed necessary for display at this zoom level. Additional items of each type can also be included based on importance determined by the Rules Engine 10180 of FIG. 3, and by implementing the weighting system of FIG. 21.

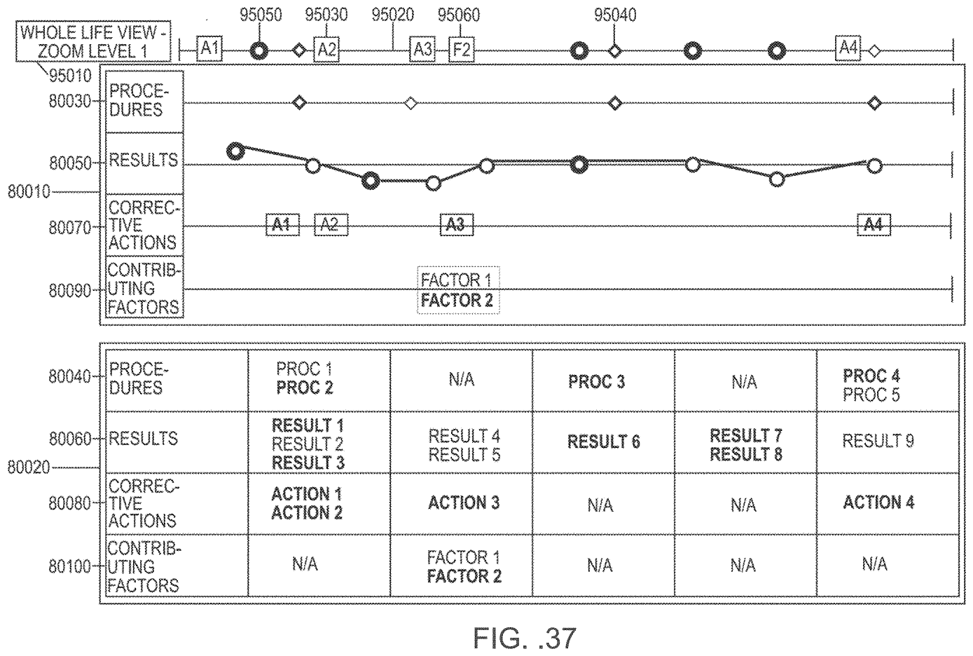

FIG. 37 illustrates a whole life view zoomed in to a first level in accordance with an embodiment of the present principles. In the embodiment of FIG. 37, Zoom Level 1 (95010) is achieved and a subsection of the entire timeline of the patient's life (95020) is displayed in more detail. This higher level of detail can be implemented when viewing any section of the Whole Life View. Underlying data (80000-80020) is reprocessed and additional key datapoints are intelligently determined for display. Configurations then apply to intelligently determine how to represent the datapoints (80000-80010). These datapoints can include additional corrective actions (95030), procedures (95040), results (95050), contributing factors (95060), and any other correlated dataset display determined for display at this zoom level.

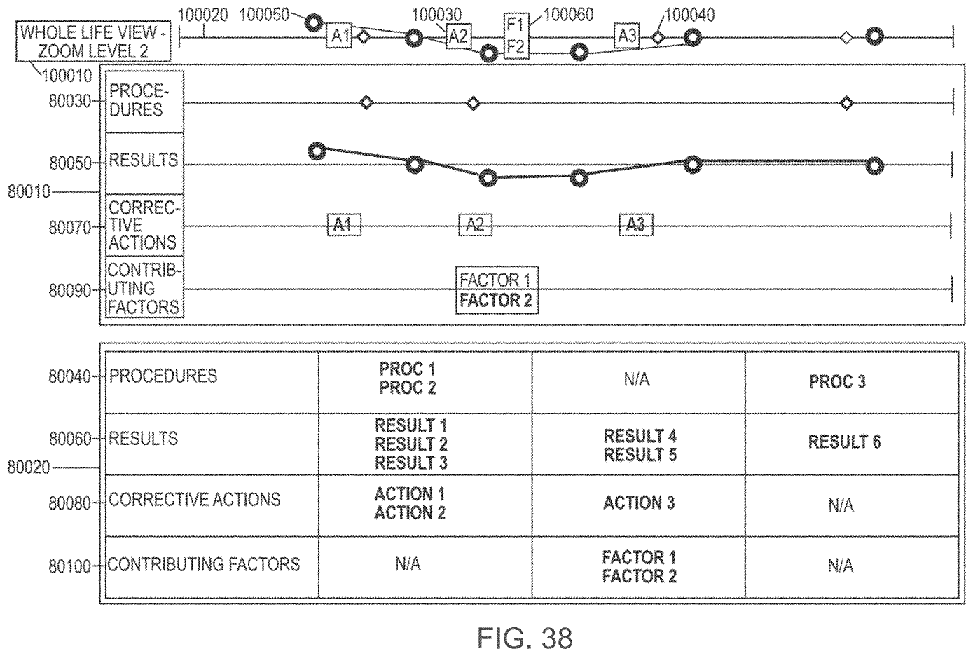

FIG. 38 illustrates a whole life view fully zoomed in in accordance with an embodiment of the present principles. In the embodiment of FIG. 38, Zoom Level 2 (100010) is achieved and a more highly zoomed subsection of the entire timeline of the patient's life (100020) is displayed in far more detail. Underlying data (80000-80020) is reprocessed and all datapoints are determined for display. Configurations then apply to intelligently determine how to represent the datapoints. These datapoints can include all corrective actions (100030), procedures (100040), results (100050), contributing factors (100060), and any other correlated dataset display determined for display at this zoom level, as mentioned before, or a new set of data can be defined based on the increased zoom level affecting weighting as defined in FIG. 21. Graphical representations can take on higher levels of resolution and accuracy (100050), include predefined colors and alerts, and may allow more discrete interaction. All predefined manner of graphical and textual formatting can be displayed.

In some embodiments, a further level of Zoom can bring a user directly into a patient-specific flowsheet. Zoom can be refocused at any time, in or out, and/or on different areas of the timeline. Events on the timeline can be interacted with in such manner as would within a flowsheet, including, but not limited to, viewing images, updating plans, viewing billing data, sending a task, setting a configuration, or any other means of interaction with which that object has been defined to accept.

Embodiments of Correlative Graphs and Whole Life Displays of the present principles provide aggregating datasets into multiple modules, intelligently correlating the modules along a common axis, each with their own, unique configurations and rules, with the ability to be independently or collectively interacted with, within the context of a patient's entire lifetime of healthcare. In embodiments of the present principles, zoom levels are not limited to a set number and can accommodate all degrees of zoom levels and multi-dimensional representations with multiple parallel representations as well as multiple perpendicular, or otherwise non-parallel representations.

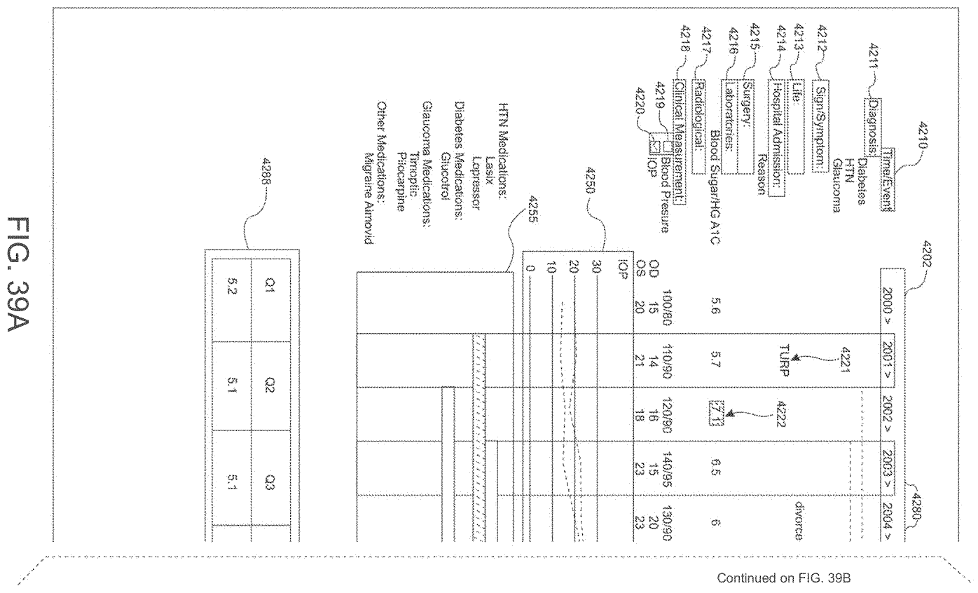

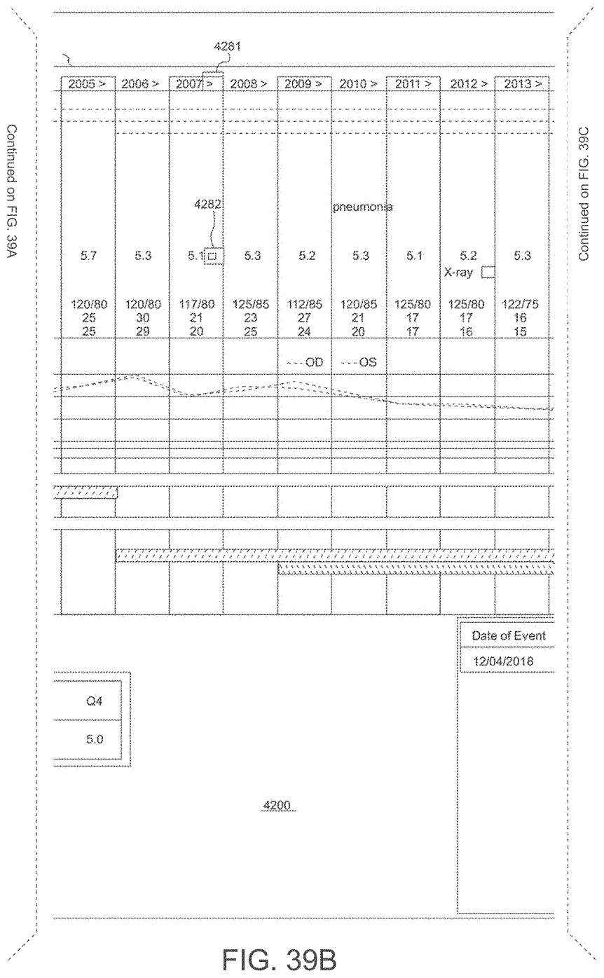

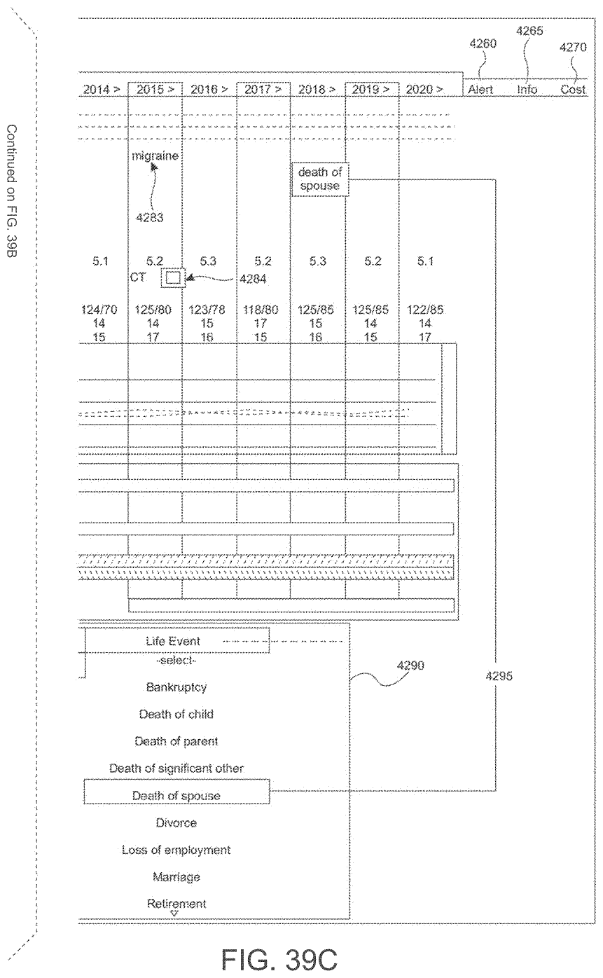

In some embodiments, the Data Command Center of the present principles, such as the Data Command center 001 of FIG. 1 enables, either as part of a medical records dashboard of the present principles or individually as a Whole Life tool, a user/medical care provider to graphically view, in a single display, a patient's entire medical history. For example, FIGS. 39A, B, and C, (Hereinafter FIG. 39) depict a graphical view of the entire medical history of a patient as a Whole Life tool in accordance with an embodiment of the present principles. In the embodiment of FIG. 39, the Whole Life tool 4200 illustratively lists dates, in one year incremented columns, across a top row 4202 of the Whole Life tool for a period of 20 years from 2000 through 2020. Although in the embodiment of FIG. 39 the time increments are illustratively one year increments, in other embodiments the time increments can be substantially any time increments chosen by the user/medical care provider.

In the embodiment of FIG. 39, the Whole Life tool 4200 in a first column 4210 lists a series of life events that occurred in a patient's life including diagnosis 4211 given to the patient, signs and symptoms 4212 the patient has had, major life events 4213 of the patient, hospital admissions 4214, surgeries 4215 the patient has had, laboratories 4216 performed on the patient, radiological procedures 4217 performed on the patient, and clinical measurements 4218 made on the patient. The Whole Life tool 4200 of FIG. 39 further illustratively includes an TOP section 4250 graphically displaying the intraocular pressure of a patient's right eye (OD) and the patient's left eye (OS) as a line graph spanning the 20 depicted years of the patient's medical history. In the embodiment of FIG. 39, the line graph of the IOP of a patient's right eye (OD) is color-coded red and the line graph of the IOP of the patient's left eye is color-coded blue for easier distinction. In the embodiment of the Whole Life tool 4200 of FIG. 39, a lower section 4260 graphically displays a medication history for the patient. In FIG. 39, horizontal bar graphs depict a history of the medication taken by and/or prescribed to a patient spanning the 20 depicted years of the patient's medical history. In the embodiment of FIG. 39, the various medication bar graphs can be color-coded to more easily distinguish between medications. In some embodiments, color standards, such as defined by the American Academy of Ophthalmology, can be used for color coding the medications. Alternatively, or in addition, in some embodiment custom colors can be used.

In the Whole Life tool 4200 of FIG. 39 any column, 4281, can be selected 4280 and expanded to take up the entire page, or a partial part of the page, or a navigation template 4290 may be used to navigate the timeline by date range or to zoom in on specific results for that time increment 4282. For example, if a user/medical provider selects the year 2007, that particular year can expand so that instead of displaying one full year as depicted in FIG. 39, the Whole Life tool 4200 can display 12 months in the year in one month increments or quarterly or in any other increments, for example, for every medical encounter the patient has had. In some embodiments, a user/medical care provider is enabled to select whether to display all the encounters that the patient has had with any medical care providers or just particular medical care providers. In some embodiments, a zoom view of a particular time span can be displayed on another monitor such that a user/medical care provider is able to view the zoomed time increment simultaneously with the whole life view.

In the embodiment of the Whole Life tool 4200 of FIG. 39, the patient illustratively had three major disease states, diabetes, hypertension, and glaucoma, as listed in the diagnosis row 4211. The Whole Life tool 4200 enables a user/medical care provider to select any of the identified major disease states to find out more detailed data regarding the selected disease state and update start/stop dates or activate/inactive a diagnosis. As depicted in FIG. 39, a user/medical care provider is able to determine w % ben the disease exactly occurred by referring to the Whole Life tool 4200. In the embodiment of FIG. 39, the diabetes occurred in 2002, 2003 was hypertension, and 2006 was glaucoma. These are chronic diseases, and these are the dates of onset. In some embodiments, the Whole Life tool can include a bar graph that can continue along a horizontal date line displaying the time period that the patient had that diagnosis, and if for some reason they no longer had that diagnosis, the bar graph could stop.

The Whole Life tool 4200 of FIG. 39 displays for a user/medical care provider in row 4212 when a patient developed a symptom and identify the symptom 4283. Similarly, the Whole Life tool 4200 of FIG. 39 is able to display for a user/medical care provider in row 4213 when a major life event that can affect the well-being of a patient occurred such as a divorce or the loss of a loved one, etc. As previously described, in row 4214, the Life tool 4200 of FIG. 39 is able to display for a user/medical care provider hospital admissions the patient had over the 20 years spanning the patient's recorded medical history. In the embodiment of FIG. 39, in 2010, the patient was hospitalized for pneumonia. As depicted in row 4215 of the Whole Life tool 4200 of FIG. 39, the patient had a surgery, transurethral resection of the prostate, in 2001. In addition, row 4216 of the Whole Life tool 4200 of FIG. 39 depicts that the patient has had laboratories, illustratively, blood sugars labs were performed, like hemoglobin A1C and update start/stop dates or activate/inactive a diagnosis. It should be noted that in the embodiment of the Whole Life tool 4200 of FIG. 39, a valued displayed in some rows and/or columns can be an average value of a measured parameter for the time increment depicted by the column. That is, in some embodiments each row and/or column can be a smart row or column and if a laboratory was taken four times in a year, the Whole Life tool 4200 can be configured to display an average of all values measured during the time increment. In some embodiments, by selecting a value in a row, patient data/information can be displayed in a window or other display means depicting all of the values measured and/or laboratories for the time increment. Even further, by selecting a particular measured value or laboratory, further detailed information for that particular value or laboratory can be displayed to a user/medical provider. Although the embodiment of FIG. 39 is described as displaying an average value, in some embodiments a high, low, or other particular value can be selected by a user to be displayed 4222 represents an alert for an abnormal result.

In row 4217 of the Whole Life tool 4200 of FIG. 39, radiological procedures performed on the patient are displayed. For example, in FIG. 39, a CT scan was performed on the patient in 2015. In accordance with the present principles, by selectin the indicator in row 4217 of the year 2105, the image of the CT scan can be displayed to the user/medical care provider. In row 4217 of the Whole Life tool 4200 of FIG. 39, clinical measurement taken on the patient can be displayed. Such clinical measurement can include blood pressures taken at each doctor's visit. In some embodiments, the results can be displayed as a number. Alternative or in addition, in some embodiments, by selecting an icon associated with the clinical measurements, a graph representing the clinical measurements over time can be displayed. 4219 and 4220 represent radiological procedures and show how they may be toggled between one, many, or all. Images may be directly accessed and viewed within context by selecting them 4284.

In accordance with the present principles, in the Whole Life tool 4200 of FIG. 39, substantially any portion of a time increment, or presentation of patient-related data/information can be selected to cause a display of a more detailed view of the selected time period/value.

In the Medications section (4255) of the Whole Life tool 4200 of FIG. 39, start dates and stop dates for each of the medications are displayed and may be interacted with in accordance with Medication Management protocols described herein.

The Whole Life tool 4200 of FIG. 39 illustratively comprises three optional columns: an alert column 4260, an info column 4265 and a cost column 4270. The alert column 4260 can be used to alert a user/medical care provider of an issue that requires further attention. In some embodiment alerts are automatically created by, for example a Rules module (described in greater detail below), and alternatively or in addition, alerts can be input by the user/medical providers with access to the Whole Life tool 4200.

Whole Life view may be interacted with whereby a doctor may choose to update an event, such as a life event (4290) by selecting said event and the event will auto-populate on the whole life view (4295).

The info column 4265 of the Whole Life tool 4200 of FIG. 39 can be used to provide information for a user/medical care provider. For example, in some embodiments, links can be provided to direct a user/medical care provider to sources of additional information, such as PUBMED, if the user/medical care provider is interested in learning about medications. Alternatively, or in addition, the info column 4265 can be used by users/medical care providers to provide information to other users/medical care providers.

The cost column 4270 of the Whole Life tool 4200 of FIG. 39 can be used to display to a user/medical care provider information associated with cost in providing medical care a patient. For example, in some embodiments, the cost column 4270 can be used to provide to a user/medical care provider information regarding what a patient's insurance company will authorize. Alternatively, or in addition, in some embodiments that cost column 4270 of the Whole Life tool 4200 can display to a user/medical care provider information regarding bills, paid or unpaid, associated with a patient.

In some embodiments, a user/medical care provider can input patient-related data/information into a Whole Life tool of the present principles. Alternatively, or in addition, a Rules module can auto-populate patient-related data/information into a Whole Life tool of the present principles. For example, in some embodiments, an integration module of the present principles, such as the integration module 002 of the Data Command Center 001 of FIG. 1, can collect patient data/information from outside sources (e.g., an EMR system). The patient data/information is made accessible, for example via a storage means, to a Rules module of the present principles, such as the Rules module 004 of the Data Command Center of FIG. 1. In addition to having access to the data/information collected by the Integration module 002, the Rules module 004 can have access to all information input by a user/medical care provider via, for example, a medical records dashboard or any other user interface. Alternatively, or in addition, in some embodiments, the Rules module 004 is configured to further have access to patient related information and general medical knowledge including but not limited to medical information regarding health conditions and treatments, symptoms and side effects, procedures, images and diagnosis, and other related medical information. As such, in some embodiments, the Rules module 004 can auto-populate at least portions of a Whole Life tool of the present principles. The Rules module 004 can then, via for example a Display module, such as the Display module 006 of the Data Command center 001 of FIG. 1, can cause the display of any portion or zoomed-in portion of a Whole Life tool of the present principles.

Embodiments of a Data Command Center convert and generate a display/view that efficiently translates clinical decision support (CDS) into a visual display. For instance, an OCT can be correlated in a display with injections, along with the measurements in microns of the OCT, graphing results on a timeline. The application renders efficient representations to effectively assist a user/medical care provider in determining the best course of action for a patient.

In embodiments of the present principles, once individual practice patterns are learned using AI/machine learning techniques, decisions can be customized. All users may not perform their practice in a similar manner, however, there are nationally set guidelines of preferred practice patterns based on condition. Based on these and other relevant information, sets of preferred practices can be programmed to guide users/medical care providers. Through evaluating local, regional, and national practice patterns, best practice can be identified and disseminated among users of embodiments of the present principles.

Although embodiments of the present principles can be applied to all fields of medicine where there are different medications and procedure options for treating any medical condition, described below is an example of a retina surgeon using embodiments of the present principles to treat Diabetic Macular Edema. Information always considered important to the treatment of Diabetic Macular Edema include, but is not limited to:

1. Type of injected medication in an eye

2. Frequency and time interval between injections

3. Impact on the swelling of the very center of the retina, called the fovea, as measured by an OCT; this center thickening measurement is called central macular thickness (CMT)

4. Clinical findings and how this impacts the patient's vision.

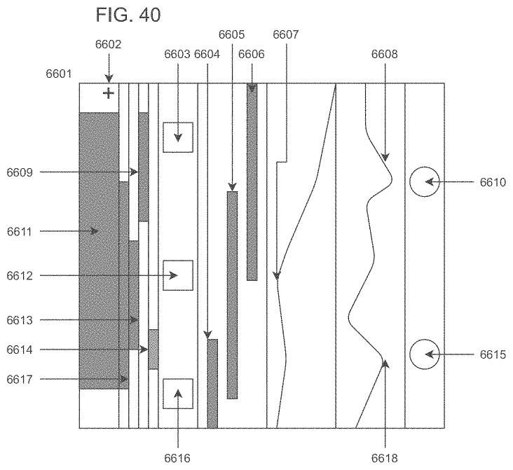

As seen in FIG. 40, several embodiments and correlations may coexist within the same, or multiple, panels. A panel such as this may be generated by selecting an icon, such as 21085 of FIG. 25, or invoked as the result of a trigger or triggers. At 6601, we see multiple, correlated panels aligned. 6611 denotes a logical grouping of medications, and may exist as a summary representation of all medications within the logical group. Employing an icon such as seen at 6602, a user may expand to show the individual medications. 6611 may represent medications for a condition, such as diabetes. 6617 may represent one diabetes medication, while 6609 may represent another, and 6609 may represent a third. It may only be important for a doctor to know the patient is being treated for diabetes, yet the option exists to drill down into the specific medications. 6603, 6612, and 6616 may represent procedures. These procedures do not necessarily require a logical grouping and may represent a heart attack at 6603, broken leg at 6612, and kidney stones at 6616. A summary representation of all procedures may be employed outside of a doctor's specialty to make them aware of the larger health of a patient. When correlated in context of diabetes medications, it may instruct a different plan of treatment. 6604-6606 may represent conditions. In the case of 6604 and 6605, conditions may start and stop, yet at 6606, a condition may be ongoing, or may represent a condition for which there is no end. Each of these conditions are correlated with medications and procedures. At 6607, we see a line graph representing results, perhaps blood sugar for a diabetic, and we specifically see a rise correlated with the onset of the condition at 6606. At 6608 and 6618, we see another line graph, perhaps representing blood pressure. The spikes in pressure correlated with life events, represented by 6610 and 6615. A death in the family at 6610 may be responsible for the spike at 6608, and 6615 may represent a marriage, with a slower rise in pressure building up to the event at 6618. The ability to correlate disparate information, otherwise not seen, correlated, or even present, in existing systems allows an overview as to the full health of a patient and continuity of care. Those skill in the art will appreciate that each module or section of each module is a dynamic data representation, and as such, may be reordered, hidden, expanded, collapsed, augmented, or otherwise altered in accordance with present principles.

The ability for Medical Records Dashboard users to customize their dashboard, without leaving the dashboard, itself, is an important step toward actualizing medical intelligence. To then share these configurations with other users is sharing medical intelligence. This invention is a tool for sharing medical intelligence. Sharing may also include the configured underlying data long with configurations.

As illustrated in FIG. 3 10180, Configuration storage is maintained through the Application Service 10150. Configurations determine the information to be displayed within the Medical Records Dashboard, as depicted in FIG. 13A-FIG. 13C, the representation of said information, as depicted in FIG. 14, and Rules employed when displaying the information, as depicted in FIG. 15. FIG. 16A and FIG. 16B illustrate examples of possible configurations. FIG. 17 illustrates Trigger configurations, and FIG. 19 illustrates storage of said configurations. FIG. 20 is an example of Configured Rule Execution, and FIG. 21 is an example of event weighting. All of these are Configurations utilized within the Medical Record Dashboard.

FIG. 29A and FIG. 29B illustrate programming said configurations on the Medical Record Dashboard. Selection of configuration parameters may be performed for a selected panel. One skilled in the art will appreciate that configurations may be entered through configuration interfaces, such as those depicted in FIG. 35A and FIG. 35B. An additional configuration option, the ability to share configurations, may also be made available. FIG. 4 illustrates a multi-tenancy architecture that supports transfer of information between tenants with a shared data access point 10220. Such information may include configurations and underlying data.

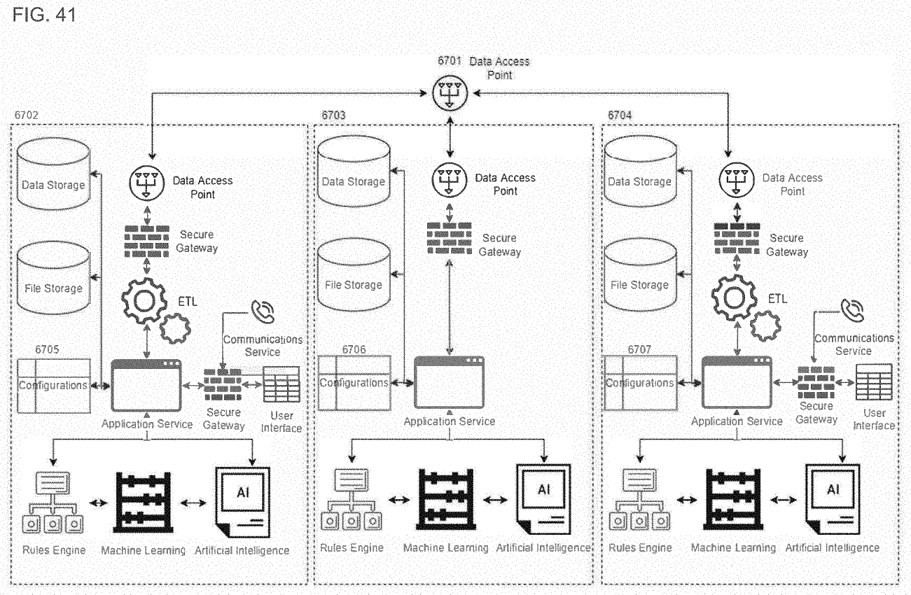

In FIG. 41, we see the shared data access point 6701 connecting multiple instances of the Medical Records Dashboard System 6702 and 6704, and a centralized storage and transfer instance 6703. Through direct sharing of data between Client 1, 6702, and Client 2, 6704, or utilizing the centralized storage repository, 6703, configurations may be stored, transferred, and shared. Client 1 configurations 6705 may be set as sharable, enabling those configurations to be stored in the centralized storage repository configurations 6706, or shared directly with Client 2 at 6707.

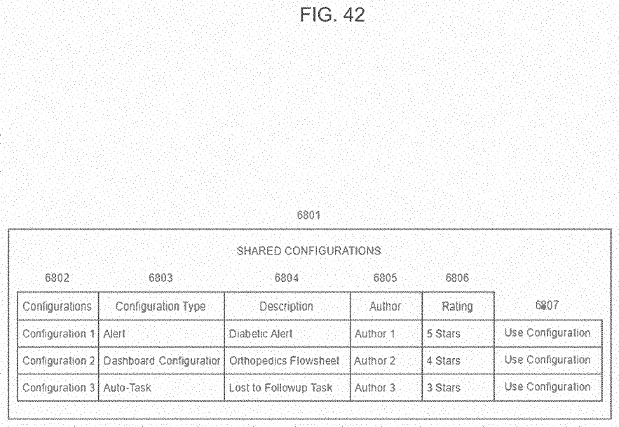

FIG. 42 depicts an example interface for selection of shared configurations. At 6801, we see a window or panel displaying shared configurations. Shared configurations may display at least one of a Configuration Identifier 6802, Configuration Type 6803, Configuration Description 6804, Author 6805, and Rating 6806. A chosen configuration may be executed by employing the Use Configuration button 6807 for a respective configuration. This would allow for the import of a shared configuration into the Client making the selection.

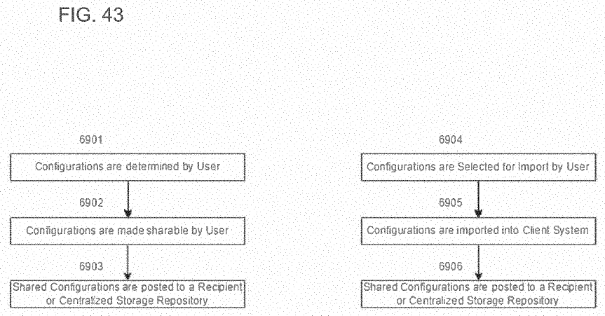

FIG. 43 illustrates two Shared Configuration workflows. At 6901, Configurations are determined by a user. Configurations are then made sharable 6902. The concluding step is the sharing of said configurations 6903. At 6904, a client selects Configurations for import into the client system. Configurations are then imported 6905. At 6906, share configurations are employed and usage is recorded. Usage may then display to subsequent clients, as depicted in FIG. 42, 6806.

In the same method of transferring medical intelligence between instances of the Medical Records Dashboard through configuration sharing, the sharing of patient records is also a function of the system. As illustrated in FIG. 4, multitenancy allows for access to data between multiple instances of the Medical Records Dashboard, 10230 and 10250. The external data sources 10210 specific to each instance are connected internally through a Data Access Point 10220. This is a method of transport for sharing of patient information. To create a link between users and data interconnected through the Medical Record Dashboard System, and those not connected through the same system, an additional step is required. The solution, in this case, is to allow for sharing of information outside the Medical Record Dashboard System, and the ability to ingest information from said outside participants. Shared care is illustrated in FIG. 13, 70240, FIG. 24A, 4404, 4406, 4410, 4412, and 4414, FIG. 25 at 21060 and 21070.

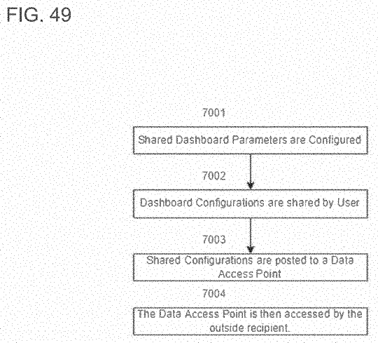

FIG. 4 accounts for external data sources 10210. Such at external data source could be a contributor not integrated with the Medical Record Dashboard System. In this case, an interface must be provided to allow for input of information. Ingestion is accounted for at the Data Access Point in FIG. 3 10120. The interface is a Medical Records Dashboard Interface, used as a Clinical Data Source 10020. The Interface, as depicted in FIG. 18, FIG. 23, FIG. 24, FIG. 25, et al is made available through the Medical Record Dashboard sharing workflow FIG. 49, through a data access point FIG. 3 10120.

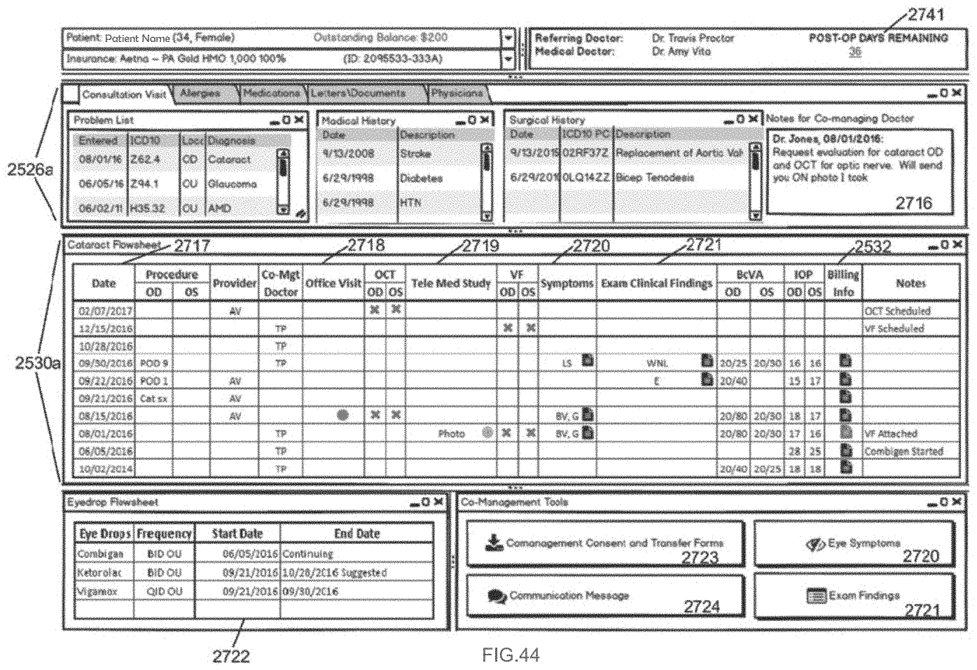

FIG. 44 depicts an embodiment of a co-managed medical records dashboard in the Data Command Center of the present principles in accordance with one embodiment In the embodiment of FIG. 44, an optometrist and an ophthalmologist share (co-manage) a patient's cataract surgery then share treatment of the patient's glaucoma. A notes field 2716 in the Consultation Visit 2526a panel presents a mechanism to facilitate contextual content surrounding the co-managed procedure(s). A Cataract Flowsheet 2530a (purpose optimized dynamic panel) is presented with structured data elements designed to facilitate the identified procedure as conducted by multiple care givers. The Cataract Flowsheet 2530a (purpose optimized dynamic panel) is presented with structured data elements designed to facilitate the identified procedure as conducted by multiple care givers. The Cataract Flowsheet 2530a is arranged by interaction dates 2717 and tracks office visits 2718 (both scheduled and realized) including sending reminders to patients and alerts when an appointment is missed (not shown), provides means to review and issue concurrence or dissent with diagnostic tests 2719, a summary of symptoms 2720, and a summary of exam findings 2721. The Data Command Center keeps track of appointments between comanaging providers and when an appointment is not kept. The Data Command Center enables messaging to both providers as well as reminders through patient portal for patient to schedule appointment. (describe modules and details of this function). Where available, billing summaries 2532 are presented in the Cataract Flowsheet 2530a as well. Clicking the billing summary 2532 can open a new billing window to show billing details. Eye drops after cataract surgery and/or glaucoma treatment can be tracked on the Eye Drop Flowsheet 2722 (another purpose optimized dynamic panel).

In the embodiment of FIG. 44, there is panel of co-management tools that provide the user with a means to download relevant forms 2723, and to send direct messages to the co-managing physician using button 2724 to access a co-management message center. An indication of the number of postop days remaining 2741 may also be provided. All financial data in the system, including costs to patient, is compartmentalized such that no user can see financial details for users or organizations not authorized in accordance with applicable policies and law. In addition, any rows and columns of information can be programmed to include or exclude those data fields from either provider.

To co-manage a patient using the interface embodiment illustrated in FIG. 44, when a referring medical care provider outside the practice wants a consultation, he or she can connect to the practice they are referring to and send information by opening the Cataract Flowsheet 2530a. The referring physician can manually insert or auto-populate information from any previous visit of the patient and provide an annotation 2717 giving the reason for the consultation. When the receiving consultation medical care provider examines the patient, the Cataract Flowsheet 2530a is auto-populated with the medical care provider's findings. Co-management forms can be downloaded from the table at 2718 either at the time of the referral or after the consulting medical care provider fills out the paperwork and the patient signs a consent form by selecting co-management forms or by visiting the referring medical care provider's website. A message is sent to the referring medical care provider to open the Cataract Flowsheet 2530a and the consent or other forms can be clicked upon and the referring medical care provider can read or sign any forms needed. The “co-management consent” can change color or be distinguished in some other fashion when received back from the referring physician. Every time the surgeon sees the patient, the Cataract Flowsheet 2530a automatically includes the date and findings. Then, post-operatively the co-managing physician, or consultation physician or optometrist, when they see the patient in their office, auto-populates or fills out on the Cataract Flowsheet 2530a and shares any results.

In the embodiment of FIG. 44, notes may be communicated between the medical care provider by selecting “communication message” 2724 to determine if there is any information that needs to be shared for office visits. The date column 2719 and office visit column 2720 are tied together. Some of the columns are left blank until the patient actually shows up for a future visit. For instance, after a surgery or consultation, the consulting medical care provider, just as they would normally give an appointment card to a patient can actually give a co-management medical summary table where it shows the date of the future appointment at the referring or sharing medical care provider's office, and when that date arrives the patient is seen and everything is auto populated so the surgeon can see the results that the co-managing medical care provider found. The findings are auto populated by the optometrist/referring medical care provider/co-managing sharing medical care provider. If the appointment date is missed, the table can link up with the missing ticket report or send an alert to the patient themselves, the surgeon, the referring medical care provider, business managers or anyone else as appropriate.

In accordance with the present principles, shared medical care may be provided in management of common eye conditions besides cataracts, such as glaucoma. For example, an optometrist/general ophthalmologist can manage interval visits after the glaucoma specialist establishes a plan of care. That is, after initial consultation, the plan can be shared with the referring or co-managing medical care provider. At a subsequent examination, the referring medical care provider accesses patient data, executes the plan and enters the data into a Cataract Flowsheet and/or a Glaucoma Flowsheet. An alert can then be sent to the glaucoma specialist confirming that the action plan is being carried out. This facilitates can care for the patient according to the plan. The glaucoma specialist can follow up every year or two while sharing interval visits with the referring optometrist/general ophthalmologist. Multiple benefits of the concepts of the present principles include excellent care, appropriate supervision, reduced cost, improved quality of care of the patient without undue distance traveled. At any point of execution of the treatment plan, treatment can be altered based on clinical data available to the patient, glaucoma specialist as well as the referring medical care provider at all times. Of course, other fields of medicine and industry have similar examples. For example, orthopedic surgeons share care with podiatrists and family physicians share care with all medical specialists. A prime example is shared care with multiple healthcare providers caring for a patient with a chronic disease, state such as diabetes. One patient can have an eye doctor, podiatrist, primary care doctor, endocrinologist, nephrologist, dietician, exercise physiologist, all who need to share care. Different medical care providers can order the same or different tests. If they are in separate health systems, they may not know each other's diagnostic tests, but through the shared medical records dashboard of the present principles, medical care providers can avoid duplication of ordering tests, thereby, reducing costs and delivering better care. In some embodiments, different practices can identify what is important for them to know about a patient and information from the various respective medical records dashboards can be combined so that the identified important information can populate into a single dashboard.

For instance, a general ophthalmologist can have a complex case, for instance neovascular glaucoma, which can sometimes be associated with carotid disease. In some instances the ophthalmologist can send the patient to a glaucoma surgeon. In some embodiments, the pertinent portions of the medical records dashboard of the general ophthalmologist's can be displayed to the glaucoma surgeon, who now has the necessary information to care for the patient. The general ophthalmologist's medical records dashboard can be automatically populated to include the encounters between the patient(s) and the general ophthalmologist, so that medical care providers can, in real time, see what the changes in the patient's treatment are made. In some embodiments, other specialist can become involved in the treatment of a patient and can also have respective medical records dashboards that can share information with some or all of the other medical records dashboards of already involved medical care providers.

In addition, embodiments of the present principles as described above can be implemented to track laboratory tests. For example, every day a family physician and the patients they see can schedule radiological or diagnostic tests to be performed on a patient. A difficulty arises in keeping track of all the different referrals and/or the medications that are prescribed. A medical records dashboard of a Data Command Center of the present principles is able to keep track of every single diagnostic test, medication, or consultation that medical care providers prescribe. Using a medical records dashboard of the present principles, a medical care provider can sort a patient's medical history by date ordered, date performed, or by patient. The results can be automatically collated in rows and columns or in other orientations on a single display. As a patient's laboratory results come back, an entire group of patients that were seen in any time period or for a particular diagnostic test can be displayed in red on a medical records dashboard until the results are received. Upon receiving the test results, the test results can turn another color to indicate the receipt of the results. In such a way, a medical care provider is able to track all of their practice's patients and what the results are, when they are received. In some embodiments, a medical care provider can be alerted to abnormal results.

In embodiments in which the Data Command Center of the present principles, such as the Data Command center 001 of FIG. 1, enables the Co-Management of patient information available via a medical records dashboard of the present principles and as described above, Co-Management is meant to refer to referrals, transfers of care, and any instance of the sharing of patient data either unidirectionally, bidirectionally, or multi-directionally between a Data Command Center of the present principles and any source of patient data and the management of such data via, for example, a medical records dashboard in accordance with the present principles. In some embodiments, a Co-Management process of the present principles can be accessed utilizing a button, keystroke, or series of keystrokes, to initiate the Co-Management workflow.

In some embodiments, upon initiation of a Co-Management process of the present principles, a user can be given the option (i.e., via a prompt on a display) to select a predetermined template for performing Co-Management, to select to determine a custom configuration for performing Co-Management, or to select a hybrid configuration for performing Co-Management. For example, in some embodiments, a template or set of templates can be preconfigured and stored and accessible to at least one of the Rules module 004 and the Display module 006 of the Data Command center 001 for configuring the medical records dashboard and displaying the medical records dashboard in accordance with a selected, preconfigured template. In some embodiments, a predetermined templates can be preconfigured based upon conditions including but not limited to a specialty of at least one medical care provider/user, practice location of at least one medical care provider/user, the identity of at least one medical care provider/user and/or at least one patient, at least one patient's conditions, procedures performed on at least one patient, risk factors for at least one patient, diagnostic results of at least one patient, future orders for at least one patient, future appointments for at least one patient, data values recorded for at least one patient, data values not recorded for at least one patient, calculated data values for at least one patient and absolute values for display. That is in some embodiments, portions, columns, and/or rows of a medical records dashboard to be displayed or hidden can be determined based on a selected preconfigured template of a Co-Management process in accordance with the present principles.

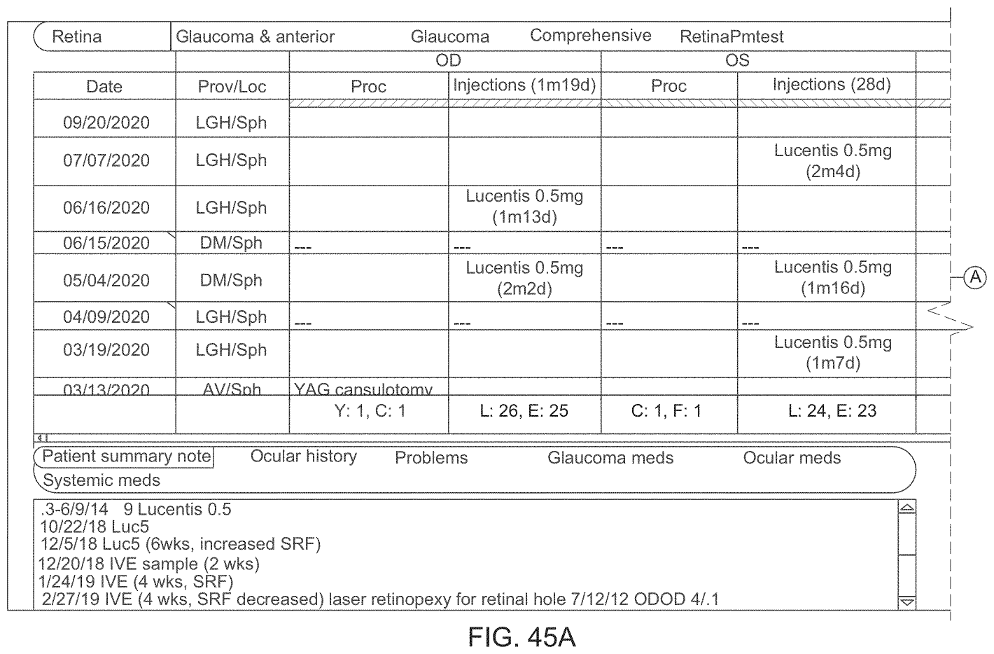

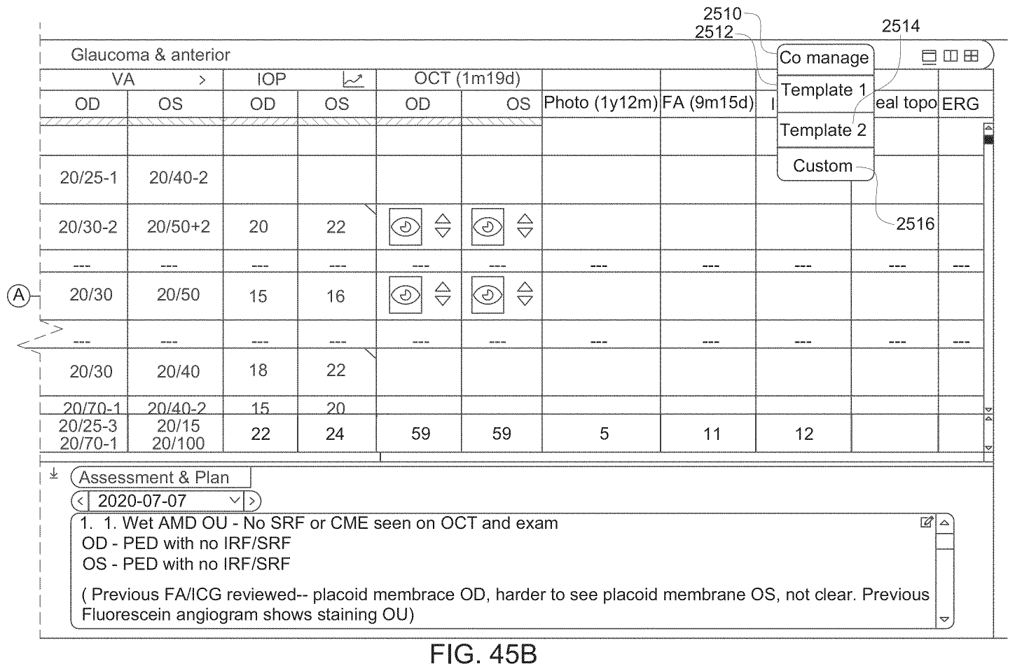

Alternatively or in addition, in some embodiments portions, columns, and/or rows of a medical records dashboard to be displayed or hidden can be determined based on a custom template of a Co-Management process in accordance with the present principles. In some embodiments a Co-management template of the present principles can be determined using, for example, a user interface of the computing device 200 of FIGS. 1 and 2. That is, in some embodiments a user interface can be implemented to create a custom Co-Management template in accordance with the present principles. For example, FIGS. 45A and 45B depict a medical records dashboard including an ability to launch a Co-Management process in accordance with an embodiment the present principles. In the embodiment of FIGS. 45A and 45B, the medical records dashboard includes a Con-Manage icon/button 2510 for launching a Co-Management process. Upon selection of the Co-Manage icon/button 2510, a menu, such as a drop-down menu, is displayed on the medical records dashboard enabling a user to select between preconfigured templates, illustratively preconfigured template 1, 2512, and preconfigured template 2, 2514. In the embodiment of FIGS. 45A and 45B, the displayed menu further enables a user to select the ability to create a custom template 2516.

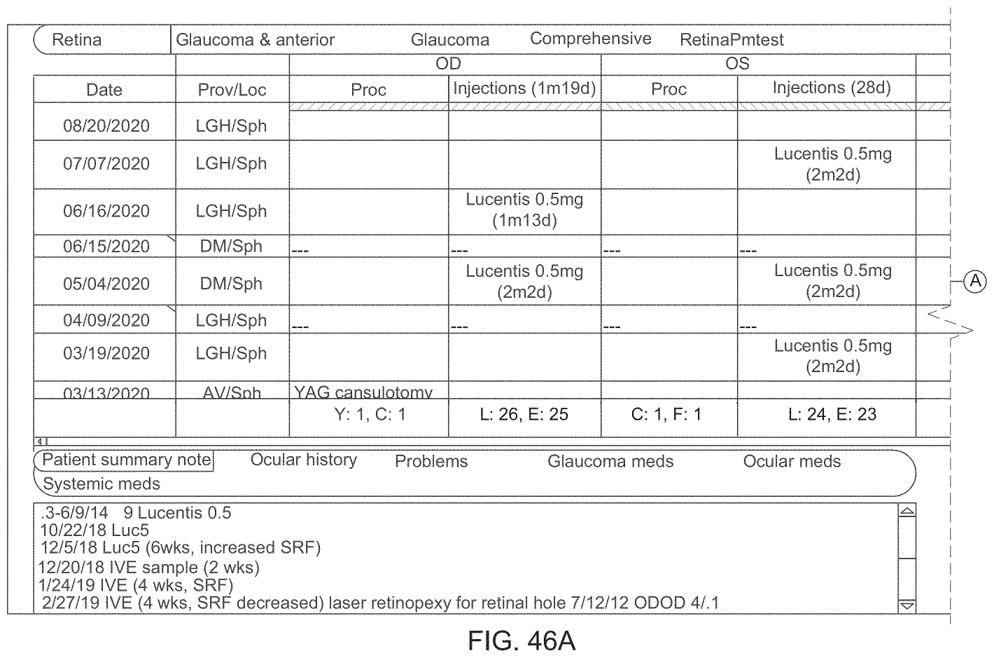

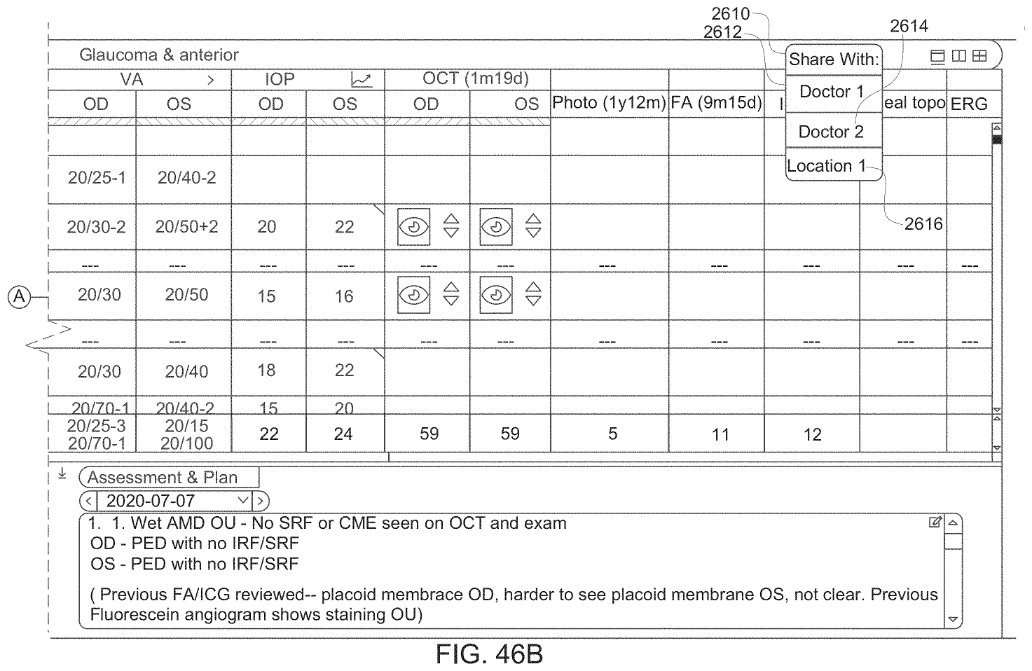

Upon selection by a user of the custom template 2516, a process is initiated that, in some embodiments, enables a user to select portions, columns and/or rows of the medical records dashboard to display or hide, for generating a display configuration. For example, FIGS. 46A and 46B depict a medical records dashboard that includes a custom template creation process for selection of configuration parameters representative of options for creation of the display configuration. In one example, multiple different display configurations may be created, allowing selection of a desired display configuration for co-management in accordance with an embodiment of the present principles.

In the embodiment depicted in FIG. 46A, a user is given the ability to select, for example using a user interface (i.e., mouse, keyboard, etc.), portions, columns, and/or rows of the medical records dashboard to be accessible to (i.e., displayed to) a shared user(s) of a Data Command Center of the present principles, such as the Data Command center 001 depicted in FIG. 1, via a medical records dashboard in accordance with an embodiment of the present principles. Alternatively, a user can select, via the process described above, portions, columns, and/or rows of the medical records dashboard to be hidden from (i.e., not displayed to) a shared user(s) of the Data Command Center.

In various embodiments, information may be highlighted or otherwise provided with an attribute in response to the information being designated as important by one or more health care providers for a patient to facilitate communication between multiple healthcare workers who may be handling different aspects of the patient's care. Clinical decision support and predictive analytics may be used to help identify events that may be important to the care of the patient. The prescribing of a new drug with possible adverse interactions or side effects may be such an event that can result in the highlighting of information related to the drug or its prescription. Such an event may also cause information that is not identified by a display configuration for display, referred to as hidden information, to be added to a panel being displayed. For example, a row may be added that is relevant to a heart specialist. A panel that is part of a patient portal may also be modified in response to such events. A missed appointment may also be displayed in the panels of several healthcare providers. The consequences of shared care may result in data relevant to each provider popping up in one of their display panels.

The ability to select options for displaying rows and columns may be provided in a table or menu format in a panel in one embodiment. Options for display of data in row selections may include text describing the options with a check boxes to enable user selection as desired to create a set of display configuration parameters. Some example options include:

```
    1 □ Row Selection
  3□ Dates
    □ Chronological
      □ Oldest
      □ Newest
  4□ Time Period
    (a) □ All
    (b) □ From _{——}c_{——} to _{——}d_{——}
  5□ Offices
    7□ All
    8□ Office 1 _{——}_{———}
      11 □ Drop down menu for more options
    9□ Office 2 _{——}_{———}
    10□ Office 3 _{——}_{———}
  6□ Providers
    16 □ Specific time periods □ Day Month Year
    12□ Include all time periods
      13□ Dr. _{——}_{———}
      14□ Providers
      15□ A □ C
      □ B □ D
    19□ By Specialization
      20□ A (Retina) □ D
      17□ Dr. _{——}_{———}
      18□ Provider
      □ A □ C
      □ B □ D
      22□ B (Glaucoma) □ E
      24□ C (Optometrist) □ F
  □ Choose circumstances when any providers' encounters the user
would want to have included and when these circumstances, referred to
as decision logic parameters, occur, any reduced or consolidated way
of displaying or emphasized way of displaying rows returns to baseline
so all rows that are affected by the circumstances appears similar reverse
a previous selection and return to baseline as appears in selection 13, 14,
19 and also can choose 28, 30, 42
  26 □ Missed Appointments □ Yes □ No
    27 □ Give details
      A □ Cancelled by patient
      B □ Cancelled by practice
      C □ No show
  28 □ Only practitioners in our practice
  30 □ Providers outside our practice
    32 □ Provider 34 □ Specialty 36 □ Other
    □ A □ C □ A □ C □ A □ C
    □ B □ D □ B □ D □ B □ D
    38 □ Practice 40 □ Hospital Admission
      □ A □ B □ C □ YES □ NO
  42 □ Share care with the above
      A □ Yes B □ No C □ Confirm
      D □ Choose particular columns to be shared
  Include:
    □ Past dates
      □ All
      □ From
    □ Today's current row
    □ Future visits
      □ Scheduled only
      □ Not scheduled
    □ Future visits
      □ Orders and plans for future
  44 □ Telemedicine Contact
  48 □ Monitoring device sending information or results
  from any entity
  A □ Laboratory or any diagnostic result
  B □ Pathology report
  C □ Life event of any kind
  D □ Home monitoring
  46 □ Patient Phone Call Encounter
  47 □ Patient Portal Contact or Similar Communication
  49 □ Office Contact Patient by any Means
  50 □ Insert Row
    51 □ Allow For Mechanism Anytime Between Row
    53 □ Clicking between any two rows
    55 □ Insert row this time period Day Month Year
  56 □ Edit Row Ability To □ Type or □ Dictate
  57 □ Insert Following _{——}_{——}_{——}_{———}
  59 □ When Inserting Row
    61 □ Columns should be as normally display
      □ Yes □ No
    □ Display only columns identified as to have
  data populated in common to both users
  □ Yes, if integrated with system
  □ No
    □ Text only to be typed one-time occurrence
  to remind of specific event that time
      200 □ User choose the following
      202 □ Confirm
        □ Yes
        □ No
```

Options specified above, and further options below enable a user to set the particular parameters for the rows (or columns that will best help them accomplish their particular job function and to integrate the encounters from those they need dates or time of encounters, essentially defining a set of display configuration parameters that can be used to create one or more panels displaying the information selected in the manner selected. The options are numbered and will be referred to in this text with a “#” preceding the corresponding number. The options allow a team working together to efficiently and accurately accomplish their individual and combined goals. It is for the user to choose their options, by first interacting and selecting options shown above, and as so doing then interacting next with options described below to complete the selection and in some cases, additional options are presented dependent on the number of options users chooses to have or the complexity of situation chosen. For instance, if there are numerous ICD 10 or CPT codes that can be chosen. This is just one example of an embodiment of the tool explaining the overall function and the choices are adjustable for each different type of field and user. Once the process of clicking options above and below and any associated drop-down options and panels to confirm the user selected, what in fact was desired and try to avoid an accidental wrong click.

#200-206 demonstrate a confirmatory process within #200 for displaying the information utilizing the display configuration parameters selected, allowing the user to actually visualize what they have selected as the options, and it sets the parameter.

#1: The row selection that enables the user to select which line or row, in one direction, is chosen. Then, once clicked, the decision then needs to be made as to how it should be displayed, so the user is taken to #2 or the options below.

The process is: First the user clicks #1, then the user looks to see which item the user wishes to select next. For instance. #3, 4, 5, 6. Once making the selection then any sub-selection that drop-down. For instance, #7, 8, 9, 10 and if there is any other drop-down menu necessary to be more specific to select, for instance under #8, which would be a particular office, if there is a certain sides to the office or other decisions to be made within a very large complex, the user would click #11. It would also come up automatically more drop-down options if programmed. This process is repeated for all of the numbers. Once the user has completed the selection in #1, the user then goes over to #2 and makes their selection in that panel on how the user wants what was selected in #1 to look.

After selecting #1, the user can decide how, if selecting #3, all the dates which are essentially in time and date order in one direction. For instance, rows would be chronological and would be the major selection. The user can go from top to bottom, oldest or newest (the tool also provides for a sorter function, so anything can be asked and only those rows that meet the criteria of the question are displayed or just those rows can be highlighted temporarily or permanently or otherwise provided an attribute to distinguish other rows. This process and option allows a user to synthesize and analyze data in a unique way going way beyond spread sheets that just summarize, add, and subtract particular columns or parts of a column, and this tool allows direct visualization and highlighting that helps the user for instance find patterns).

#4 defines a time period that the user wants the tool or dashboard to display. Is it from the beginning of time that is recorded and there is data? If so, the user selects #4A. So, the user chooses all or is it from a certain period of time #4B and the user can select in #4C a beginning then in #4D a conclusion. This is a typical method of a calendar coming up where the user can select a day, month, or year or it can be typed in or it can be any mechanism to help the user to insert a date or time, but all options can be allowed in this spreadsheet. Even dictating it and speaking through voice recognition.

#5: Shows offices and if that is selected, the user is choosing a location. The location can be where they work or all offices of a practice. This can be customized to each entity that uses the tool. This example #7 shows the selection “all offices.” The user can select #8 and choose just one office or additional offices #9 and #10. The other offices, some users may have just two offices, others might have 30. After selecting a particular office, #11 shows an example of where there can be another panel that would open up within an office. There could be many more options. If it is a hospital system using this tool, #11 could be very extensive because it might be floors, departments, or even multiple locations within a hospital in one particular geographic radius.

#6: Numerous providers can be selected. #12 allows the user to select which time period the spreadsheet should populate, which could be from the beginning that the patient has been in the practice starting in the practice, which is likely most common, so if all #12 is selected and #13 allows entry of a particular user, could be the signed in user, so tool would, if #13 is selected, populates this particular doctor. Then, how that doctor wants their personal row displayed is chosen in the options below. Next #14 allows the choice of populating the spreadsheet with more encounters from other providers and user selects as many as desired. If the choice of time period to populate is different then, #16 allows, a specific time period, and #17 allows that time period to be for just certain doctor or #18 allows the user to select from many providers, by specialization after the user has selected. When selecting #12 explains all time periods from the beginning when the patient started to whatever is in the data to the future. #16 allows the user to provide a level of specificity. Several other options allow similar specificity level selection. In particular, #16 allows the user to specify a specific time periods of day, month, year or it could be time in a day. As in a hospital admission, patients can be examined 24 hours in a day. There could be 24 rows. If a scientist is doing research a second or microsecond may be chosen as the time element or astronomers may choose time in 1000 year increments.

Clicking #13 can know this is being set for just one particular doctor or provider. If it is not just one particular provider #13 lists just the doctor or provider and a drop-down menu can occur with many different providers.

#14: Allows the user to select exactly which providers that are using the table. #15 gives an example of A but this can be as many providers as necessary. The user can first click one or several or chose them all. It all depends on who is using the tool and who is setting it. If it is just one doctor setting for himself or a group of doctors, all getting together to set, what they all want to see that is one way the tool can be easily set. But, setting for a large health system or corporation with many thousands of employees takes more consideration. Who the providers are and how they are categorized can, in this large corporate or even international corporate entity, be very complex. The tool accommodates all options from small personalized organization to large complex international entity are entirely different.

#16: Again, allows the user to select a specific time period. The day, month, or year. When selecting again #17 can be specified for which doctors. #18 again, selecting a group of doctors. #12 is all time periods. #16 the user is actually specifying what time period the tool is sorting, selecting and presenting the rows and options below allows the option of how it is to be displayed.

#19: Is by specialization. #14 is more particular doctors chosen, but #19 is grouping of doctors. When choosing a group there are examples of just a few that the tool would know are specialization. By way of example, if the specializations is just eye doctors, the tool would then prompt options A, B, C, D, E, and F choices of eye doctors. #20 being retina, #22 glaucoma, #24 optometrists, all types of eye doctors, and so on and so forth. If it is for a large health system or otherwise, where there are many different options. All options #19, A, B, C, D, E, F and so on can be any type of specialist. Orthopedic surgeons, dermatologists, or if it is outside of medicine, it could be different types of lawyers in a law firm. Those handling civil cases, divorce, real estate, patent, business, corporate, etc.

#26: Allows for missed appointments. This is more of a catch-phrase term. If it was an appointment that was supposed to occur for whatever reason is displayed and even the reason could come up by hovering or other means, using the example of a patient. The next section would be giving details.

#27: When hitting details there can be many different reasons that can roll over or other reasons or display in different ways. For instance, #27A is cancelled by patient. #27B is cancelled by practice. #27C is a no show. All of these might have different reasons. If a patient continues to no show for an appointment, instead of cancelling in advance, that might want to be displayed differently, practices cannot fill scheduled slots if patients do not cancel in advance. How it would be displayed is selected in #2. But, how it is categorized and show up as a row is determined by the checked boxes.

#28: Selected if the rows should only be within the practice and were selected above. For instance, #30 shows providers outside the particular practice using or setting the spreadsheet as shown in the options above and below. If the user is only interested in the user's own legal firm, accounting firm and following on a table what the user personally has done and sub dividing all the different lawyers working together in the user's firm, does the user really care what lawyers on the outside do? Also, when those outside entities saw a client, does the user's firm care-perhaps or perhaps not, but the tool allows the user to select that choice. In a field like medicine this could be critical to see encounters outside the users own interaction with the patient. While the practitioner might not work within the user's entity, a patients heal this interrelated and even if the patient always comes to the user's large health system, what if the patient happens to visit, somewhere 1000 miles away, and has a major medical event, even though the provider is not within the user's practice, of course a doctor would need to know because when the patient comes back, it might affect their treatment of that patient. Even if in the same city, the patient goes to different hospitals and different doctors, all can be informed as to what the other provider does or discovers about a patient, as they may be inter-related. Therefore, certain users of this tool might want to see all of a sub-set of the different providers, even if they are not related to the direct practice.

#32: Allows the user to select a particular provider.

#34: Allows selection of a certain specialty.

#36: Can be any category. User needs to designate for them to maximize use of the tool.

#38: Can allow selection of all the providers in a certain practice or entity, A, B, or C are examples of entities outside user's own practice, but no matter what provider is working in the particular practice A, B, C, or D, they may want to see certain rows of other users selected.

#40: Hospital admissions. Is it to be included? Could also be emergency room or any type of sites or issue. User setting the criteria or other methods in other embodiments would respond yes or no in A and B. Again, how this is displayed each time would take the user to the below options, how to display for selected rows.

#42: Question is, does the user want to share care with the above? Here the user says, yes or no. #42 D allows the user to click and the user can choose particular columns to be shared. This takes the user to an entirely new panel (not depicted), which goes into great detail to choose what information to share between two entities that may be sharing the same tool. For instance, they may have a dashboard, spread sheet and a second practice that is filling and selecting the usage of the tool may also have a spread sheet. Both can share those spread sheets, both can insert rows into each other, but which rows are going to be shared and how are they going to be displayed would be #2. But, here D represents a whole other panel that comes up. This would require both confirming they will accept the others rows and columns or perhaps only allow read only access or just one user accessing the others. The analogy is similar to the need to opening both doors in two adjoining hotel rooms to allow entry for both. Only certain rows or columns can be selected to be shared. In this case, since only one provider is filling this out and allowing certain information to come over into their spread sheet. The reverse is not true, unless it has been arranged to be that way and the other user allows this. When there are the shared rows or spreadsheets and depending on the industry regulations, would be followed with necessary permissions obtained and confirmed by the tool before allowing entry. Example in health care, it would require patient record release and sharing approval with HIPAA rules followed.

#44: Allows, if doctors communicate on the phone, telemedicine or any which way that, or a provider talks to another provider at a certain time and wants to leave a row for when that happens, and what was said can be submitted there as a row.

#46: Patient phone call encounter. In medicine there are usually only rows for actions when the doctor sees the patient or performs an examination. In reality, currently every time a patient makes contact, and asks a question about a symptom or a problem, doctors do not even know the call occurred unless they are contacted and something important can easily be missed. Same with #47. There could be a message through the portal. When the patient had the symptom or the question can be important. This tool allows an option #46 and #47 to insert in a row. #49, if the office contacts the patient, that might need to have a row. But, how that is displayed, of course, is very different than any other row. That is why after hitting each of the row selection columns, the user is brought over how to display the selected rows in the options below. In a case like this #46 and #47 might not be always a relevant as #13. For example, the user might choose to hide, make collapsible, barely visible, just a line with color, or other attributes. Knowing that there is something there and it can be hover over and expanded is maybe what is important.

#47: The user can select in #47, the monitoring device, which sends information results from any entity. An extensive drop-down menu is presented as monitoring or diagnosing can mean virtually anything and the user can decide what is important for them to display. One embodiment actually captures the ability of the computer to help select what's important in a particular row, based on how the doctor has told the system they want information to be displayed. It does so which can occur, without human interaction. In some cases, if the user has selected, in the system, to not include certain rows of certain providers, there are certain programs and circumstances that are informative and there are certain programmed circumstances where the information still may be displayed because somethings the information is so urgent can also be allowed.

Spreadsheets usually require significant human entry and interaction. This tool is significantly autopopulated, although it can be manually populated. But, no matter how data is entered, whether automated by computer or human interaction and entry, what has been displayed and how time and date is presented has equal presentation as mentioned above. This tool can measure critical events even if it's not in an encounter of the user itself. For instance, if the user wants to see laboratory results, pathology specimens, radiological studies, many of these require human intervention to interpret. Yet, it's lost somewhere in the computer. This tool can automatically timestamp when the result or event occurs. It can put in a row, as designated by the user, how it should look in chronological order or any other matter within the dashboard. These outside events can be on a different portion of the dashboard not necessarily chronological, but it is up to the user and the way it's displayed, which impacts differently, so it catches the user's attention until it's looked at and opened. The most common embodiment has the presentation in the rows when the results come back or when the test is performed. So, while the rows can just represent user interaction of health care practitioners to a patient, they can just as easily be the result

#48: Brings up a drop-down menu. This represents monitoring techniques or devices and sending information results from any entity. In all fields, there can be incredibly important attachments or events that may be unrelated to a particular time that the user actually interfaces with the client or in the case of health care with the patient. Doctors may place orders and the orders for certain diagnostic tests to discover disease, such as radiological studies, blood tests, etc. could be done alone on an entirely different day than a normal row, which has been selected. For instance, in #13, and #14, when the user actually interfaced or took an action, with or without a client, or in this case a patient, the test or any other means of gathering information may be completed on a certain day or time. To make certain this is not missed, it could be organized in many different ways. It can be set to be attached to the rows the next time a user sees the client or patient or it can be inserted chronologically when completed. By way of example, if a doctor sees a patient in September of 2019 and schedules the patient, again, one month later, in November 2019 but orders three different tests that are done on three different day's and the studies come back in October, all three dates in October that they were either performed or the results come back can be inserted in a row. Then, when the doctor sees the patient in November, they will see the rows that are displayed, which will be selected from options below. It can be clicked on, get the results, and then turn off what had been an alert that was set in the options below to highlight more significantly. Once read, it perhaps becomes less significant by hitting another display option below.

#48A is an example of diagnostic tests. #48B could be pathology reports, and #48C could be major life events of any kind In reality, especially in a field of healthcare, if a patient has a sudden death of a loved one, this can impact every thing else. The date of that death, because of a sudden car accident, could impact the health of the patient in even way Therefore, knowing the date, by putting it in a row, whether manually or automatically, if it happens to be an event that can be collected automatically, if not put in manually, may impact other dates of service and other treatments or plans of action going forward. #48D gives an example of home monitoring In the field of medicine, patients are now able to check their blood pressure or other items at home. If there is a major occurrence while measuring blood pressure that is out of the normal standard for that patient, that high or low event could be placed in a row so when the doctor interfaces with the spreadsheet they can see what happened. Clearly, how this role presents needs to be very different then a major surgery event or a major encounter by a doctor. Hence, why this tool and only the stool allows the user to differentiate items in time and date as shown in the options below.

Once the selections are completed in #1, it immediately takes the user one by one to the how to display for selected rows, discussing this in depth. Once something is selected in #1 the user can make the selection of #70. It will select #70 and the issue is one time only so the user is making a particular selection on a particular row in this case and it is only one time. Perhaps it is just a certain day, month, or year #72. It is a time period from a certain day, month, or year #74. Display until the patient returns for the next visit, so it is only a temporary situation. But, the user can choose the temporary situation being the treating provider, which could be the primary provider, #13 that modified the tool for their use. When the patient returns only to that provider, that could be an option to only display until that provider (#76) opens or sees the encounter or #78 could be kept open until any provider in the practice next encounters the patient so, the row remains highlighted as chosen below until then. #79 allows it to remain open and not disappear until a doctor clicks it and it is certain it has been seen and only then highlighted method chosen turns off.

#200 and #202: Allow confirmation of any step or selection.

Options for how to display selected rows are now shown:

How To Display For Selected Rows

```
  70 □ One Time Only □ Day Month Year
  72 □ Time Period □ Day Month Year
  74 □ Display Until Next Pt Visit by:
  76 □ Treating Prov. 78 □ Any Prov in Practice
  79 □ Keep open until it is open
    80 □ Permanent 83 □ Only from _{——}_{——} to _{——}_{———}
  84 □ Normal Size
  86 □ Smaller 2X, 3X, _{——}_{———}
  88 □ Larger 2X, 3X, _{——}_{——}_{——}
  89 □ Strike Out
  90 □ Color
      □ Red
      □ Yellow
      □ Other _{——}_{———}
  91 □ Bold
  92 □ Lighter
  93 □ Hide
  94 □ Unhide (all actions can be reversed
  individually or reset for a gray)
  94 □ Collapse
  95 □ Move
  109 □ Make Editable
        110 □ Select editable capabilities
  97 □ Allow Ordering Panel Access
        98 □ Select ordering options
  99 □ Allow Action to do with One Click
  □ Pull Up Underlying Data/Image
  100 □ Allow rollover mechanism
  101 □ Window mechanism link
  102 □ Allow changes in row from
  original presentation when clicked on the row
  120 □ Override mechanism - this takes the user to other choices
  when the above are overridden, i.e., certain situations or diagnosis
requires resetting of spreadsheet entirely, i.e., development of disease
like rubeosis triggers need at moment of diagnosis for two different
providers to now equally have rows displayed
  110 □ Option Repeat for columns (other direction)
        111 □ Select here for the choices in how to select
  the columns
          112 □ In all cases
          113 □ Only when integrating with outside
  Practice
          103 □ Choose any option 84-95
```

#80: Allows whatever had been selected in #1 to be permanently displayed.

#82: Allows just a certain time period for it to be displayed. The beginning and the end can be selected.

#79: Keeps displaying it until it is opened by a provider. That way, whatever, was the important row that was submitted and inserted has to be opened by someone. It can even be made permanent at that time or who opens it might matter.

#80 is a permanent selection for the rows. #82 is just taking the action for certain time period.

#84: Selecting this option sets whatever was selected in #1 is inserted in rows at the normal size, which is the most common choice, or instance, #13 or #14. But, when doing special considerations, for instance #30, and #46 or other examples, clearly that needs to be differentiated and the user would be unlikely to choose #84. That is where the specialization selection process that follows will come into effect. #86 allows any row or group of rows, as defined, to be smaller any size can be chosen. It can be half size, one third size or other.

#88 allows it to be a larger row, two times or three times. Also, any of these rows can be from a certain time period. Option #79 can keep it that size until it is open. The concept is something needs to be larger because it is important one time until red or acted upon, then it can go back to a different size. When opening any row at one time, that particular row can be adjusted at the time it is seen and acted upon. #88 can enlarge the column and in all cases the time period and which rows are to be acted upon are selected.

#89: Strike out so it is dashed in between the line.

#90: Choose any color, as seen for instance as it is normally found: Red, yellow or other.

#91: Make previously selected rows defined and selected above bold. That is true with #70 through #103.

#92: Lighten or have row fade out.

#93: Hide

#94: Collapse

#95: Move

#96: Make editable. This is a very important feature. It allows the user to actually edit a row and save it and be able to write anything in this row by clicking option. This takes the user to an entirely other option where how the user make something editable, what the user can do, what the user can save, how to save it, where the user can copy from other areas to put something into this row can all be achieved.

#97: Allows ordering panel access. This means that any particular row or a cell within the row, as an intersection in a column could allow and activate a mechanism to open up an ordering panel that would allow a user to make an order and allow for other functions to occur because something happened in the row. This, again, when the user hit #110 would take the user to an entirely new panel that will allow for the types of ordering and options and what type of options to create in the row. By way of example, if the row happens to display a diagnostic test or does not display a diagnostic test, because it is lacking an order for a future diagnostic test could be started by hitting this particular row.

#98: Allows for action to be done with one click on anything on that row and that can be set.

#99: Allows the mechanism to pull up data that is found in a row. This could be anything from a diagnostic test or an image to something written or more information and numbers.

#100: Allows a roll-over mechanism. On that row, there can be more information and simply rolling-over or any aspect of the row and/or column or intersection would pop something up.

#101: Pulls up a window mechanism as well as an option for a link within that row for anything that is set in that row. This can take the user from one row to another location to gain more information on a subject matter on the row.

#102: This will allow changes in a row from the original presentation or selection, so when clicked, that particular row can be manipulated any which way even overriding what had originally been set. Only certain users can do this and it may just be user based because on user can make a choice of a change in one row that other users, who use the same dashboard, might not agree with.

#103: Choose any option #85 to 95. This is done because if the user chooses #102 where the user can make changes in any row, the user might want to highlight that row in some way. It will then, allow the user to make an option by choosing #103. Any option the user wants.

#103: The user selects here for the choices of how to select the columns.

#110: Allows the user to make selections and changes for the other direction in the dashboard. For instance, the columns. To start the action, hit #111.

#112: The user is making these options more permanent.

#113: Only when integrating from outside of the practice, the columns might be selected. It is important to note that this particular option to repeat for the columns for whatever the other direction is not being expounded upon because dashboards do have many options that are in existence to choose in one direction only how a column should look (but they do not have options to do the other direction). This tool allows the user to, in one direction, perhaps columns make the typical changes that allow insertion of columns on a dashboard. This tool allows the other direction which are not set to be set.

In some embodiments in which a user selects to create a custom template, upon selection of the creation of a custom template, the Rules module 004 can initiate a process, for example as described above with reference to FIG. 46A, for enabling a user(s) to select to which to portions, columns, and/or rows of the medical records dashboard a user(s) is to be allowed or denied access. In some embodiments the Rules module 004 stores such custom template configuration selected by the user(s) in a storage means accessible to the Rules module 004 and the Display module 006. Upon creation of a custom template for Co-Management in accordance with the present principles, the Display module 006 can cause the display or lack of display of portions, columns, and/or rows of the medical records dashboard based on the determinations and information associated with a created, custom template.

In addition to the selection of a preconfigured template, for example preconfigured template 1, 2512, and preconfigured template 2, 2514, and/or the creation of a custom template, for example custom template 8716, in some embodiments, a Data Command Center of the present principles, such as the Data Command Center 100 depicted in FIG. 1, via a medical records dashboard, can enable a user(s) to select parameters that decide to whom/what/where to enable access or deny access to portions, columns, and/or rows of the medical records dashboard. For example, in the embodiment of FIG. 46B the medical records dashboard comprises a Share With menu 2610 enabling a user(s) to select to whom/what to enable access or deny access to portions, columns, and/or rows of the medical records dashboard. In some embodiments, the Share With menu 2610 can include predetermined selections such as a first doctor, Doctor 1 2612, a second doctor, Doctor 2 2614, and a location, such as the location of a medical practice, Location 1 2616. Alternatively or in addition, in some embodiments the medical records dashboard can enable a user(s) to input identifying information including but not limited to a specialty of at least one medical care provider/user, practice location of at least one medical care provider/user, the identity of at least one medical care provider/user and/or at least one patient, at least one patient's conditions, procedures performed on at least one patient, risk factors for at least one patient, diagnostic results of at least one patient, future orders for at least one patient, future appointments for at least one patient, data values recorded for at least one patient, data values not recorded for at least one patient, calculated data values for at least one patient and absolute values for display to identify to whom/what to enable access or deny access to portions, columns, and/or rows of the medical records dashboard.

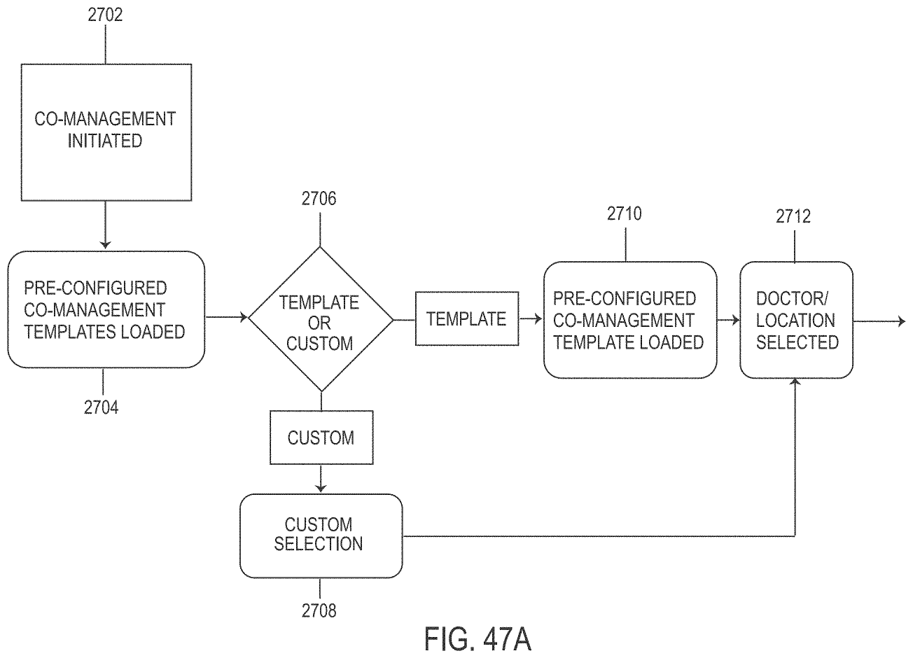

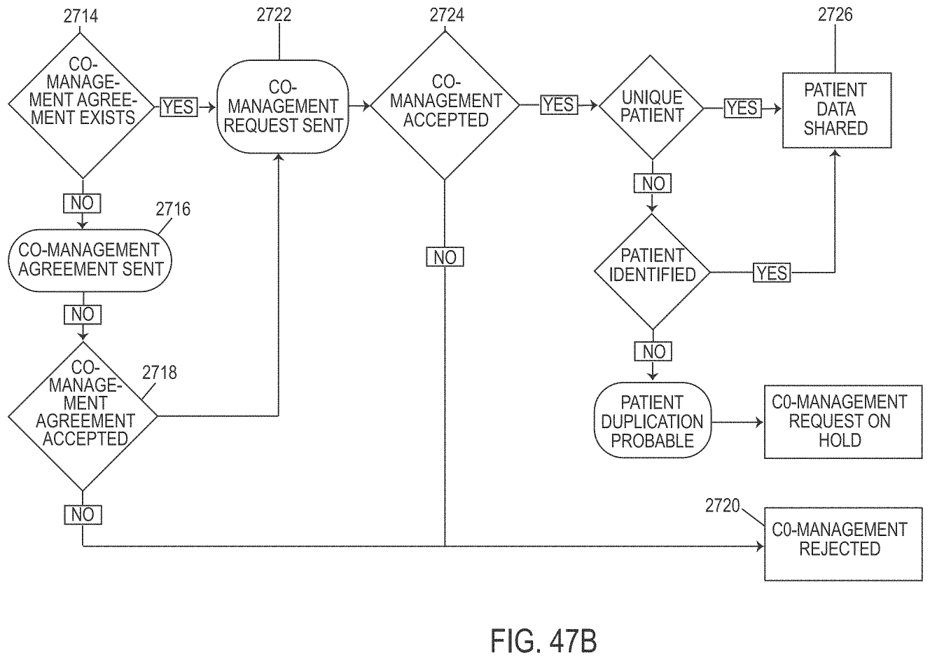

FIG. 47A depicts a first portion of a workflow diagram of a Co-Management process and FIG. 27B depicts a second portion of the workflow diagram of the Co Management process of FIG. 47A in accordance with an embodiment of the present principles (referred to collectively as FIG. 47 herein). In the embodiment of FIG. 47A and FIG. 47B, the Co-Management process is initiated at 2702. At 2704 preconfigured Co-Management templates are all loaded. A selection is then made by a user(s) to use a pre-configured template(s) or to use the Custom option to create a Custom Co-Management configuration at 2706. If a user selects to use a pre-configured template, the selected pre-configured template is loaded at 2708. If a user selects to create a Custom Co-Management configuration, user selections for creating the Custom Co-Management configuration and determining which portions, columns, and/or rows of the medical records dashboard to which to grant or deny access are made at 2710. In the embodiment of FIG. 47A and FIG. 47B, at 2712, a user selects select to whom/what/where to enable access or deny access to portions, columns, and/or rows of the medical records dashboard.

At 2714 it is determined if a Co-Management agreement exists. If no Co-Management agreement exists a Co-Management agreement is communicated to at least one other user at 2716. At 2718 it is determined if the communicated Co-Management agreement was accepted by another user. If the communicated Co-Management agreement was not accepted by another user, the Co-Management agreement is cancelled at 2720. If at 2718 it is determined that the communicated Co-Management agreement was accepted by at least one other user, a Co-Management request is communicated to an accepting user at 2722.

Referring back to 2714, if it is determined that a Co-Management agreement does exist, the process also proceeds to 2722 during which a Co-Management request is communicated to at least one user with which the Co-Management agreement exists. At 2724 it is determined if the Co-Management request was accepted. If at 2722 it is determined that the Co-Management agreement is not accepted, the Co-Management agreement is cancelled at 2720. If at 2722 it is determined that the Co-Management request has been accepted by at least one user, the patient data is shared at 2726 in the medical records dashboard in accordance with the pre-configured template selected or the custom configuration created and the whom/what/where selections made by a user(s).

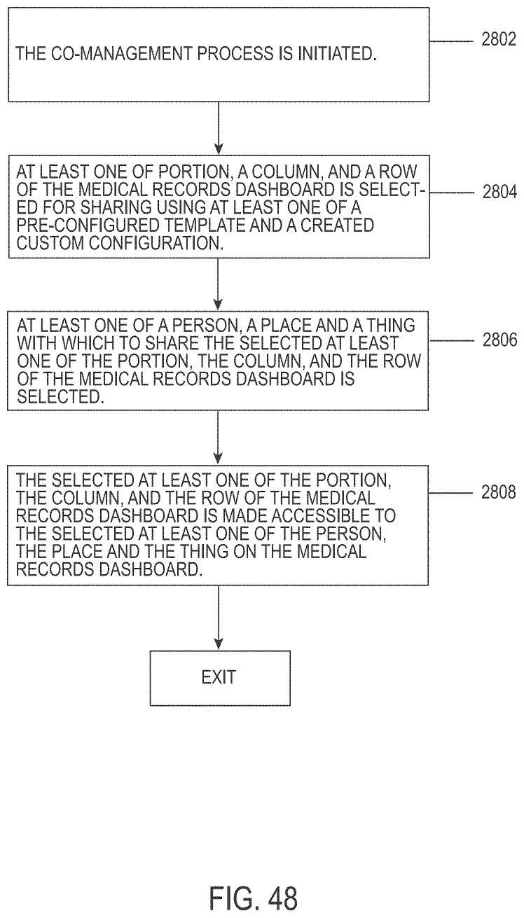

FIG. 48 depicts a flow diagram of a method for Co-Management of patient information in a medical records dashboard in accordance with an embodiment of the present principles. In the embodiment of FIG. 48, the method begins at 2802 during which the Co-Management process is initiated. For example and as described above, in some embodiments the medical records dashboard can include a Co-Management icon/button for initiating a Co-Management process in accordance with the present principles. The method can proceed to 2804.

At 2804, at least one of a portion, a column, and a row of the medical records dashboard is selected for sharing using at least one of a pre-configured template and a created custom configuration. The method can proceed to 2806.

At 2806, at least one of a person, a place and a thing with which to share the selected at least one of the portion, the column, and the row of the medical records dashboard is selected.

At 2808, the selected at least one of the portion, the column, and the row of the medical records dashboard is made accessible to the selected at least one of the person, the place and the thing on the medical records dashboard. The method can then be exited.

In some embodiments, the Co-Management Workflow can exist in a single, unidirectional state, whereby the party that initiates the Co-Management request shares data with the recipient, but the recipient does not reciprocate sharing of patient data. In another embodiment, the party that initiates the Co-Management request shares patient data with the recipient, and the recipient initiates a Co-Management request to the party that initiated the initial request, thus data is shared bidirectionally. In another embodiment, several parties initiate Co-Management requests, and each party shares data with each other party, in a multi-directional state. At any point, a Co-Management participant my opt to no longer share data with one or more recipients, at which point data sharing and the Co-Management workflow reaches a logical end.

In some embodiments, upon initiation of Co-Management, a record of the Co-Managed patient is recorded, including all relevant Patient Identifiers from all parties involved in Co-Management. Alternatively or in addition, upon initiation of Co-Management, shared configurations are recorded. Shared configurations can be used to determine what data from each party can be viewed within a recipient's medical records dashboard in accordance with the present principles.

In some embodiments, a source of patient data can exist within storage means associated with respective Data Command Centers of users participating in the Co-Management of the present principles. In such embodiments, shared data can consist of a series of links or cached data in the respective Co-Management databases. Links or cached data can be updated upon any change in source. Additionally in some embodiments, data can be recorded within a Co-Management database as well as a database/storage means associated with a participating user's respective Data Command Center, the data including, but not limited to, audit logs of Co-Management Workflow interactions, Messaging between users, file and document sharing between users, and notifications and/or triggers for automated tasks. It should be noted that, in some embodiments, a Co-Management Workflow in accordance with the present principles can be non-linear, can be automated in whole or in individual or groups of steps, and algorithms can intelligently update, flag, or otherwise override certain steps of the Co-Management Workflow.

In one example of a Co-Management Workflow in accordance with the present principles, a primary care physician (PCP) can initiate the Co-Management Workflow for a single patient having multiple Specialists. Each Co-Managing Specialist can opt to Co-Manage with one of more PCPs and Specialists. In some embodiments, the Co-Managed patient data would not be shared further than one logical step, thus a PCP can share their patient data with Specialist 1, who then shares their patient data with Specialist 2, but the PCP's patient data would not be shared with Specialist 2 unless the PCP takes action to initiate Co-Management with Specialist 2.

In a second example, a doctor can initiate a Co-Management Workflow of the present principles with a patient during a Transfer of Care, in which case, the patient's data is shared unidirectionally, and the recipient is not expected to share data back with the initiating doctor, nor is there an expectation that the patient would return to the transferring doctor.

In a third example of a Co-Management Workflow of the present principles and with the context of a hospital and several physicians, as is normally the case in patient care, any number of Co-Management Agreements and Workflows can be in place to allow for patient data sharing between any to all recipients of a Co-Management Request. This configuration can include unidirectional sharing, bidirectional sharing, and multi-directional sharing of patient data in accordance with the present principles.

In FIG. 49 at 7001, a Medical Records Dashboard User may configure the Medical Record Dashboard, or sections thereof, to be sharable. Sharable portions of the Dashboard are then shared by the User 7002. Shared configurations are posted to a data access point 7003. The data access point may be accessed by an authorized user 7004. This eliminates any requirement for outside users of the Medical Records Dashboard to have any further access than the ability to connect to the secure access point.

The methods and processes described herein may be implemented in software, hardware, or a combination thereof, in different embodiments. In addition, the order of methods can be changed, and various elements can be added, reordered, combined, omitted, or otherwise modified. All examples described herein are presented in a non-limiting manner. Various modifications and changes can be made as would be obvious to a person skilled in the art having benefit of this disclosure. Realizations in accordance with embodiments have been described in the context of particular embodiments. These embodiments are meant to be illustrative and not limiting. Many variations, modifications, additions, and improvements are possible. Accordingly, plural instances can be provided for components described herein as a single instance. Boundaries between various components, operations and data stores are somewhat arbitrary, and particular operations are illustrated in the context of specific illustrative configurations. Other allocations of functionality are envisioned and can fall within the scope of claims that follow. Structures and functionality presented as discrete components in the example configurations can be implemented as a combined structure or component. These and other variations, modifications, additions, and improvements can fall within the scope of embodiments as defined in the claims that follow.

In the foregoing description, numerous specific details, examples, and scenarios are set forth in order to provide a more thorough understanding of the present disclosure. It will be appreciated, however, that embodiments of the disclosure can be practiced without such specific details. Further, such examples and scenarios are provided for illustration, and are not intended to limit the disclosure in any way. Those of ordinary skill in the art, with the included descriptions, should be able to implement appropriate functionality without undue experimentation.

References in the specification to “an embodiment,” etc., indicate that the embodiment described can include a particular feature, structure, or characteristic, but every embodiment may not necessarily include the particular feature, structure, or characteristic. Such phrases are not necessarily referring to the same embodiment. Further, when a particular feature, structure, or characteristic is described in connection with an embodiment, it is believed to be within the knowledge of one skilled in the art to affect such feature, structure, or characteristic in connection with other embodiments whether or not explicitly indicated.

Embodiments in accordance with the disclosure can be implemented in hardware, firmware, software, or any combination thereof. Embodiments can also be implemented as instructions stored using one or more machine-readable media, which may be read and executed by one or more processors. A machine-readable medium can include any mechanism for storing or transmitting information in a form readable by a machine (e.g., a computing device or a “virtual machine” running on one or more computing devices). For example, a machine-readable medium can include any suitable form of volatile or nonvolatile memory.

Modules, data structures, and the like defined herein are defined as such for ease of discussion and are not intended to imply that any specific implementation details are required. For example, any of the described modules and/or data structures can be combined or divided into sub-modules, sub-processes or other units of computer code or data as can be required by a particular design or implementation.

In the drawings, specific arrangements or orderings of schematic elements can be shown for ease of description. However, the specific ordering or arrangement of such elements is not meant to imply that a particular order or sequence of processing, or separation of processes, is required in all embodiments. In general, schematic elements used to represent instruction blocks or modules can be implemented using any suitable form of machine-readable instruction, and each such instruction can be implemented using any suitable programming language, library, application-programming interface (API), and/or other software development tools or frameworks. Similarly, schematic elements used to represent data or information can be implemented using any suitable electronic arrangement or data structure. Further, some connections, relationships or associations between elements can be simplified or not shown in the drawings so as not to obscure the disclosure.

A practice consists of a practice management system, EHR, patient engagement system, patient tracking systems, inventory management system (medications), collections system, and more. Not all of these systems talk to each other in a meaningful way. An Enterprise solution is required to connect the dots and consists of a single system with modules designed specifically for each role within a business, front desk, technician, doctor, scribe, biller, collector, et al, even down to the patient, to give the user precisely the information they need in order to perform their function. Additionally, there are executive modules designed for C-level employees or owners that give them high level summaries of what is happening in the practice, where there are bottlenecks, and suggestions on how to improve performance.

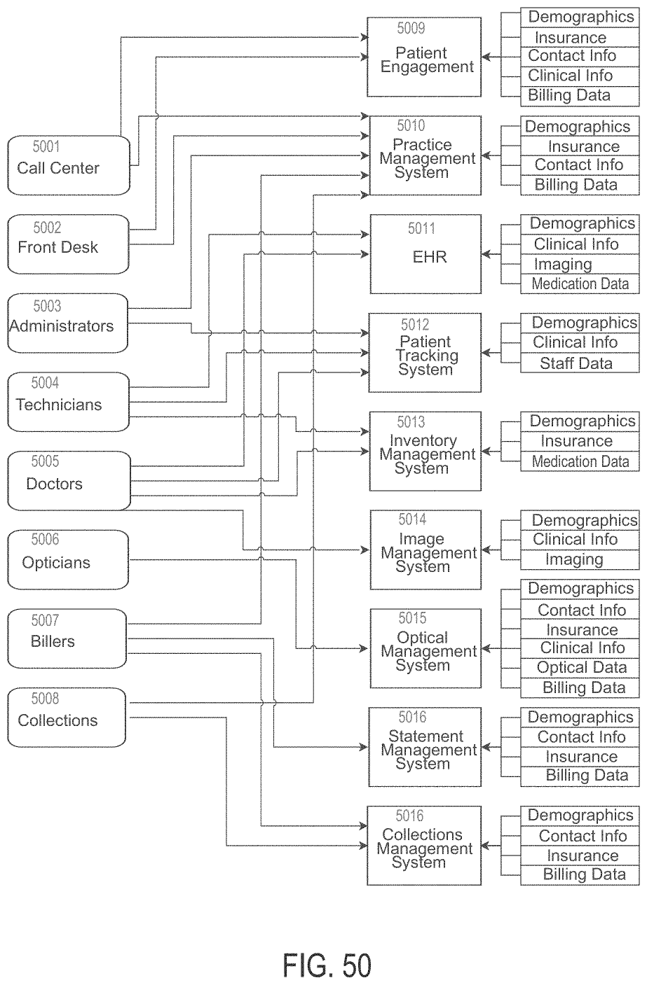

Several roles exist within any medical practice, and several computer systems exist to support the business FIG. 50. At 5001, we depict a call center, or those people responsible for patient telecommunications. At 5002, we depict the front desk, or the staff which physically interacts with the patient when they first arrive. At 5003, administrators refers to staff managing high-level practice processes. At 5004, technicians refers to the staff performing the initial patient workup. At 5005, doctors refers to the medical practitioner or practitioners that perform medical services. At 5006, opticians refers to the staff that oversees medical devices. At 5007, billers are the staff that sends the charges to insurance companies. At 5008, collections refers to those responsible for reclaiming unpaid debts. Each role has specific responsibilities, each may have a dedicated system to perform their role, and all require access to information that exists across the entirety of the systems employed by the practice.

These systems are depicted with the relevant shared data as 5009 patient engagement, 5010 practice management system, 5011 EHR, 5012 patient tracking system, 5013 inventory management system, 5014 image management system, 5015 optical management system, 5016 statement management system, and 5017 collections management system.

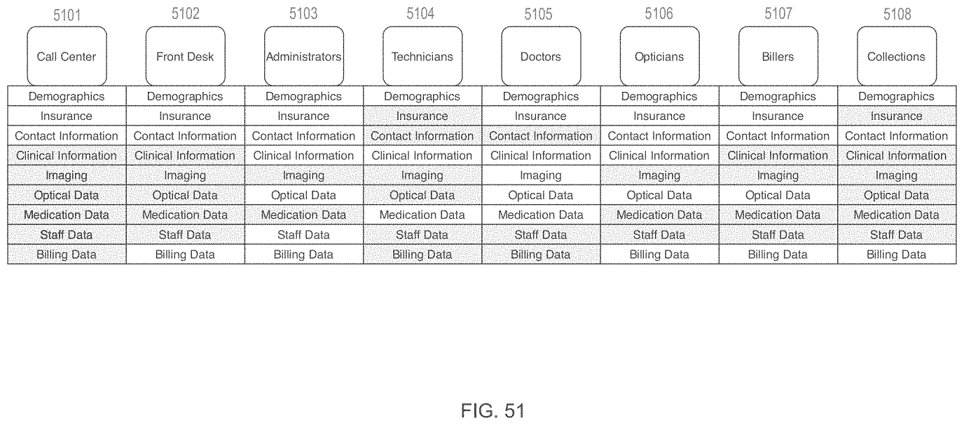

The process of utilizing multiple systems to track different aspects of the patient, patient care, billing, and communications, causes undue overlap and duplication of data FIG. 51, leading to discrepancies and limiting the ability of user access to and interaction with data outside of their assigned role. At 5101, we see the call center utilizes demographics, insurance, and contact information. Front desk 5102, administrators, 5103, opticians 5106, and billers 5107, all share these data points. Technicians 5104, doctors 5105, and collections 5108, all use a portion of this data. Several data points are shared across multiple roles, as depicted in white, shared, and gray, not shared, between each role.

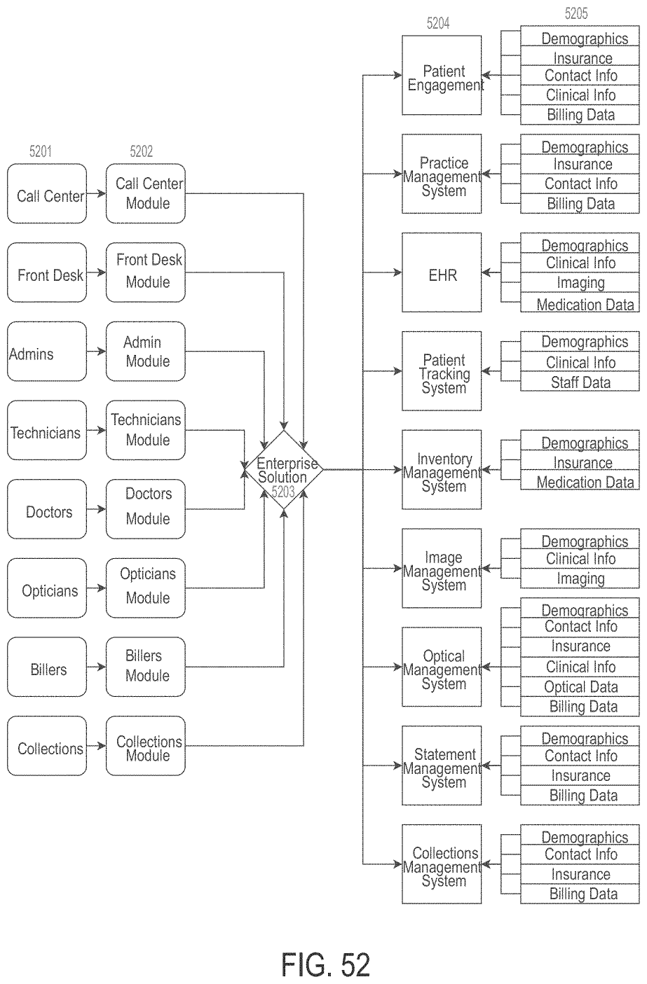

In an exemplary embodiment FIG. 52, all relevant source data is consumed from the master systems used to house said data. Said data is compared and transformed, as required, such that the output to the individual modules is precisely what that role requires to perform their role. Discrepancies between source systems are indicated by any means. Important information that exists within systems that is not normally included in the specific role's module may be added, expanded, or indicated by any means.

At 5201, we see each role, at 5202, we see a collective offering for each role, at 5203, we see an enterprise solution managing all transference of data, at 5204, we see each of the source systems data is consumed from, and at 5205, we see a list of data elements shared between systems.

The enterprise solution not only consumes data from all source systems, it also writes back, through bidirectional communication methods like, but not limited to, APIs. In one example, a doctor that notices a misspelling in a patient name, currently would have to speak to the front desk or administrator to update the patient's name. With the enterprise solution, bidirectional communication supports automatic or approval-based updates from any approved user. In another example, a doctor may create and send a task to the front desk to schedule an appointment. In such an example, the front desk may be alerted by any means, either through their module, such as on a dashboard, or by text, email, phone call, or any other means. In another example, a practice administrator may view, through a module, summaries of relevant, correlative data from the source systems. Such data may be in rows and columns, in a graph, or visualized by any means, and may interact with said data by sending tasks to relevant employees, or by setting triggers to create an auto-task. In depth charts and graphs may be visualized through the enterprise solution, including, but not limited to, an accurate tracing of patients and staff within the office. Any and all representations of data may be alerted or indicated, by any means, and may have triggers set to create automatic tasks, and may offer decision support based on relevant data.

1. A computer implemented method of creating display configurations, the method comprising:
generating, via a medical record dashboard system, a dashboard display comprising at least a first visible panel of one or more visible panels having selected information corresponding to information stored in one or more medical record databases related to different respective medical services;
displaying a configuration menu associated with the first panel;
receiving display configuration parameters selected by a first user for a first display configuration in response to user interaction with the display configuration menu;
configuring the first panel based on the first configuration;
storing the display configuration parameters of the first configuration;
receiving a selection of the first display configuration by a second user via a configuration sharing panel;
retrieving the first display configuration;
obtaining data defined by the first display configuration; and
applying the first display configuration to a second panel selected by the second user.

2. The method of claim 1 wherein the first display configuration is stored in and retrieved from an access point.

3. The method of claim 2 wherein information for the second panel is accessed from the access point.

4. The method of claim 1 wherein the first and second users are users of a single integrated dashboard system.

5. The method of claim 1 wherein the first and second users are users of separate non-integrated dashboard systems.

6. The method of claim 1 and further comprising:
accessing information for display in the second panel in accordance with the first configuration;
creating a webpage comprising the second panel; and
providing a link to the webpage to the second user.

7. The method of claim 6 and further comprising:
accessing the webpage via the link; and
displaying the webpage for viewing by the second user

8. The method of claim 7 wherein the second user is a user of a non-integrated separate system.

9. The method of claim 1 wherein the display configuration parameters specify rows or columns of a table.

10. The method of claim 1 wherein the display configuration parameters include a time range parameter for specifying a range of time for the information.

11. The method of claim 1 wherein the display configuration parameters include specification of display attributes.

12. The method of claim 1 wherein the display configuration parameters include a sharing parameter for specifying a recipient of the information.

13. The method of claim 12 wherein the recipient comprises a user, an office location, or a specialty.

14. The method of claim 1 and further comprising automatically displaying information that is hiding in accordance with the first configuration in response to selected events.

15. The method of claim 14 wherein the selected events include information designated by the first user as important or urgent.

16. The method of claim 1 and further comprising

17. The method of claim 1 wherein retrieving information comprises retrieving the information as a function of information associated with the first panel.

18. The method of claim 1 and further comprising dynamically updating the first and second panels in response to modification of information.

19. A computer implemented method of creating display configurations, the method comprising:
generating, via a dashboard system of a first user, a dashboard display comprising at least a first visible panel of one or more visible panels having data corresponding to different respective medical services;
displaying a configuration selection menu associated with the first panel;
receiving a selection of a first display configuration having display configuration parameters specifying a first set data to display and a format for the first set of data to display in response to first user interaction with the display configuration menu;
retrieving information corresponding to the first configuration as a function of the display configuration parameters;
storing the display configuration parameters of the first configuration;
receiving a request for display of a new panel in accordance with the first display configuration by a second user via a configuration sharing panel;
retrieving the first display configuration;
obtaining the first set data defined by the first display configuration; and
applying the first display configuration to display the first set of data in the new panel.

20. A computer implemented method of creating display configurations, the method comprising:
generating, via a dashboard system of a first user, a dashboard display comprising at least a first visible panel of one or more visible panels having data corresponding to different respective medical services;
displaying a configuration selection menu associated with the first panel;
receiving a selection of a first display configuration having display configuration parameters specifying a first set data to display and a format for the first set of data to display in response to first user interaction with the display configuration menu;
retrieving information corresponding to the first configuration as a function of the display configuration parameters;
storing the display configuration parameters of the first configuration;
receiving a request for display of a new panel in accordance with the first display configuration by a second user via a configuration sharing panel; and
displaying the first set of data formatted in accordance with the first display configuration in the new panel.
